(19) United States
(12) Patent Application Publication
(10) Pub. No.: US 2026/0362893 A1
(43) Pub. Date: Nov. 12, 2026

(54) COMPOSITION FOR OPTOELECTRONIC DEVICE, AND ORGANIC OPTOELECTRONIC DEVICE AND DISPLAY DEVICE

(71) Applicant: SAMSUNG SDS CO., LTD., Yongin-si, Gyeonggi-do (KR)

(72) Inventors: Yunsoo KIM, Suwon-si, Gyeonggi-do (KR); Kipo JANG, Suwon-si, Gyeonggi-do (KR); Hansol SEO, Suwon-si, Gyeonggi-do (KR); Dong Min KANG, Suwon-si, Gyeonggi-do (KR); Hyung Sun KIM, Suwon-si, Gyeonggi-do (KR); Minseok SEO, Suwon-si, Gyeonggi-do (KR); Ho Kuk JUNG, Suwon-si, Gyeonggi-do (KR); Sunghyun JUNG, Suwon-si, Gyeonggi-do (KR)

(21) Appl. No.: 19/114,968

(22) PCT Filed: Sep. 22, 2023
(86) PCT No.: PCT/KR2023/014501

§ 371 Date: Mar. 25, 2025

(30) Foreign Application Priority Data
Sep. 27, 2022 (KR) 10-2022-0122719

(51) Int. Cl. H10K 50/12; C07D 209/82; C07D 251/24; C07F 15/00; H10K 85/30; H10K 85/60; H10K 101/00

(52) U.S. Cl. CPC H10K 50/12; C07D 209/82; C07D 251/24; C07F 15/0086; H10K 85/346; H10K 85/622; H10K 85/654; H10K 85/6574; H10K 2101/90

(57) ABSTRACT

Disclosed are a composition for an organic optoelectronic device, and an organic optoelectronic device including the same, and a display device, the composition including a first compound, a second compound, and a third compound, wherein the first compound is represented by Chemical Formula 1, the second compound is represented by Chemical Formula 2 or a combination of Chemical Formula 3 and Chemical Formula 4, and the third compound is represented by Chemical Formula 5. The details of Chemical Formula 1 to Chemical Formula 5 are as defined in the specification.

### TECHNICAL FIELD

A composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device capable of implementing a high efficiency and long life-span organic optoelectronic device.

Another embodiment provides an organic optoelectronic device including the composition for an organic optoelectronic device.

Another embodiment provides a display device including the organic optoelectronic device.

### Technical Solution

According to an embodiment, a composition for an organic optoelectronic device includes a first compound, a second compound, and a third compound, wherein the first compound is represented by Chemical Formula 1, the second compound is represented by Chemical Formula 2 or a combination of Chemical Formula 3 and Chemical Formula 4, and the third compound is represented by Chemical Formula 5.

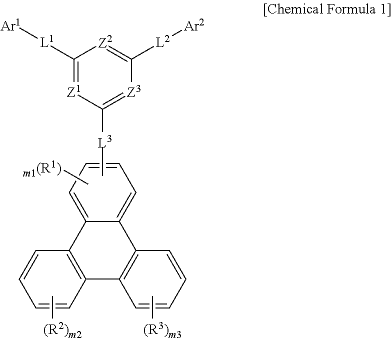

In Chemical Formula 1,
Z¹ to Z³ are N or C-L^{a}-R^{a},
at least two of Z¹ to Z³ is N,
L^{a} and L¹ to L³ are each independently a single bond, a substituted or unsubstituted C6 to C20 arylene group, a substituted or unsubstituted C2 to C20 heterocyclic group, or a combination thereof,
Ar¹ and Ar² are each independently a substituted or unsubstituted C6 to C30 aryl group, a substituted or unsubstituted C2 to C30 heterocyclic group, or a combination thereof,
R^{a}, and R¹ to R³ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
R^{a}s are each independently present or are linked to Ar¹ or Ar² to form a substituted or unsubstituted heteroaromatic polycyclic ring,
R¹ to R³ are each independently present or adjacent groups thereof are linked to form a substituted or unsubstituted heteroaromatic polycyclic ring,
m1 is one of integers of 1 to 3, and
m2 and m3 are each independently an integer of 1 to 4;

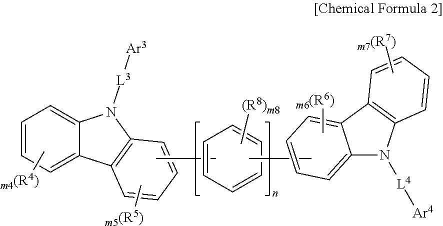

wherein, in Chemical Formula 2,
Ar³ and Ar⁴ are each independently a substituted or unsubstituted C6 to C30 aryl group or a substituted or unsubstituted C2 to C30 heterocyclic group,
L³ and L⁴ are each independently a single bond or a substituted or unsubstituted C6 to C20 arylene group,
R⁴ to R⁸ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
m5 and m6 are each independently one of integers of 1 to 3,
m4, m7, and m8 are each independently one of integers of 1 to 4, and
n is one of integers of 0 to 2;

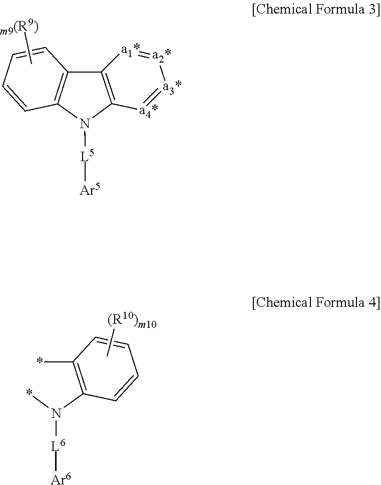

wherein, in Chemical Formulas 3 and 4,
Ar⁵ and Ar⁶ are each independently a substituted or unsubstituted C6 to C30 aryl group or a substituted or unsubstituted C2 to C30 heterocyclic group,
a₁* to a₄* in Chemical Formula 3 are each independently a linking carbon (C) or C-L^{b}-R^{b},
among a₁* to a₄* in Chemical Formula 3, two adjacent ones are linked to * in Chemical Formula 4,
L^{b}, L⁵, and L⁶ are each independently a single bond or a substituted or unsubstituted C6 to C20 arylene group,
R^{b}, R⁹, and R¹⁰ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group, and
m9 and m10 are each independently one of integers of 1 to 4;

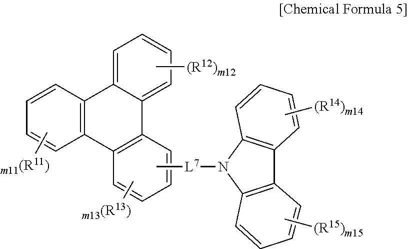

wherein, in Chemical Formula 5,
R¹¹ to R¹⁵ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
L⁷ is each independently a single bond or a substituted or unsubstituted C6 to C20 arylene group,
m11, m12, m14, and m15 are each independently one of integers of 1 to 4, and
m13 is one of integers of 1 to 3.

According to another embodiment, an organic optoelectronic device includes an anode and a cathode facing each other, and at least one organic layer between the anode and the cathode, wherein the organic layer includes the aforementioned compound for the organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a cyano group, a C1 to C30 alkyl group, a C1 to C10 alkylsilyl group, a C6 to C30 arylamine group, a C6 to C30 arylsilyl group, a C3 to C30 cycloalkyl group, a C3 to C30 heterocycloalkyl group, a C6 to C30 aryl group, or a C2 to C30 heteroaryl group. In specific example of the present invention, the “substituted” refers to replacement of at least one hydrogen of a substituent or a compound by deuterium, a cyano group, a C1 to C20 alkyl group, a C6 to C30 arylamine group, a C6 to C30 aryl group, or a C2 to C30 heteroaryl group. In specific example of the present invention, the “substituted” refers to replacement of at least one hydrogen of a substituent or a compound by deuterium, a cyano group, a C1 to C5 alkyl group, a C6 to C20 arylamine group, a C6 to C18 aryl group, a dibenzofuranyl group, a dibenzothiophenyl group, a carbazolyl group, or a pyridinyl group. In specific example of the present invention, the “substituted” refers to replacement of at least one hydrogen of a substituent or a compound by deuterium, a cyano group, a methyl group, an ethyl group, a propyl group, a butyl group, a C6 to C20 arylamine group, a phenyl group, a biphenyl group, a terphenyl group, a naphthyl group, a triphenyl group, a fluorenyl group, a dibenzofuranyl group, a dibenzothiophenyl group, a carbazolyl group, or a pyridinyl group.

In the present specification, “unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to an embodiment is described.

The composition for an organic optoelectronic device according to an embodiment is a mixture including three types of compounds, specifically, a first compound having electronic characteristics, a second compound having hole characteristics, and a third compound having buffer characteristics.

The third compound is a compound having a wide HOMO-LUMO band gap including both the HOMO-LUMO band gaps of the first compound and the second compound, and has a lower hole mobility than the hole mobility of the second compound having the hole characteristics, which slows down the hole injection characteristic and can bring about an effect of reducing hole traps.

In addition, as the third compound has lower electron mobility than the electron mobility of the first compound, and the light emitting layer region relatively shifts toward the hole transport auxiliary layer, exciton quenching at the interface with the electron transport auxiliary layer and deterioration thereby may be reduced, and thus makes it possible to increase the life-span.

The first compound having the electronic characteristics has a structure in which triphenylene or a triphenylene derivative is substituted with a nitrogen-containing 6-membered ring and is represented by Chemical Formula 1.

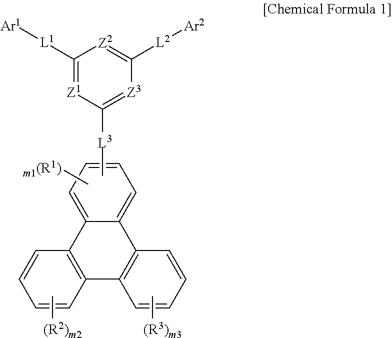

In Chemical Formula 1,
Z¹ to Z³ are N or C-L^{a}-R^{a},
at least two of Z¹ to Z³ are N,
L^{a} and L¹ to L³ are each independently a single bond, a substituted or unsubstituted C6 to C20 arylene group, a substituted or unsubstituted C2 to C20 heterocyclic group, or a combination thereof,
Ar¹ and Ar² are each independently a substituted or unsubstituted C6 to C30 aryl group, a substituted or unsubstituted C2 to C30 heterocyclic group, or a combination thereof,
R^{a}, and R¹ to R³ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
R^{a}s are each independently present or are linked to Ar¹ or Ar² to form a substituted or unsubstituted heteroaromatic polycyclic ring,
R¹ to R³ are each independently present or adjacent groups thereof are linked to form a substituted or unsubstituted heteroaromatic polycyclic ring,
m1 is one of integers of 1 to 3, and
m2 and m3 are each independently one of integers of 1 to 4.

When m1 is 2 or more, each R¹ may be the same or different.

When m2 is 2 or more, each R² may be the same or different.

When m3 is 2 or more, each R³ may be the same or different.

For example, Z¹ to Z³ in Chemical Formula 1 may each be N.

For example, Z¹ and Z² may be N and Z³ may be C-L^{a}-R^{a}, wherein L^{a} may be a single bond and R^{a} may be linked to the adjacent R² to form a substituted or unsubstituted heteroaromatic polycyclic ring, for example, a substituted or unsubstituted benzothiophenpyrimidine.

For example, Z¹ and Z³ may be N and Z² may be C-L^{a}-R^{a}, wherein L^{a} may be a single bond, and R^{a} may be linked to the adjacent R¹ to form a substituted or unsubstituted heteroaromatic polycyclic ring, for example, a substituted or unsubstituted benzothiophenpyrimidine.

For example, Chemical Formula 1 may be represented by any one of Chemical Formula 1-1 to Chemical Formula 1-3.

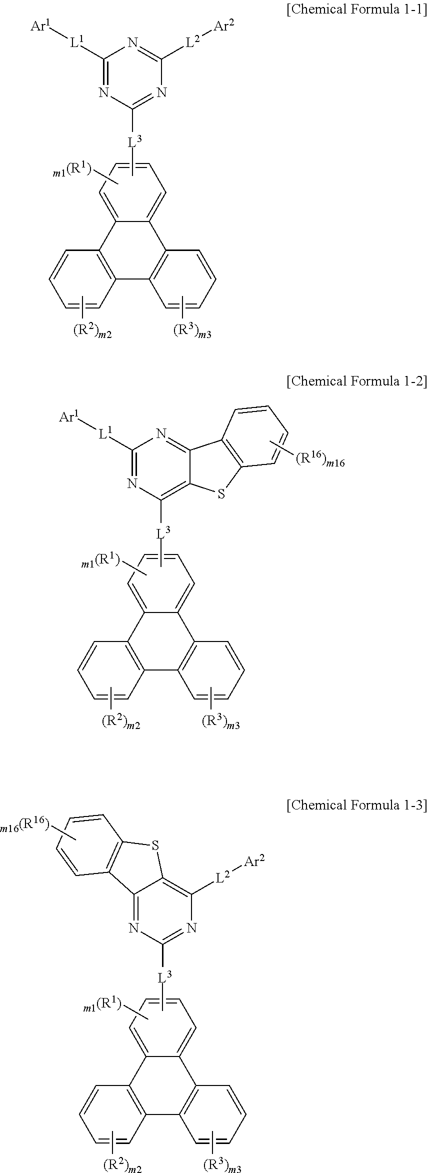

In Chemical Formula 1-1 to Chemical Formula 1-3, the definitions of R¹ to R³, L¹ to L³, Ar¹, Ar² and m1 to m3 are the same as described above,
R¹⁶s are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and
m16 is one of integers of 1 to 4.

When m16 is 2 or more, each R¹⁶ may be the same or different.

In an embodiment, each of R¹⁶s may each independently be hydrogen, deuterium, a substituted or unsubstituted C1 to C10 alkyl group, or a substituted or unsubstituted C6 to C12 aryl group.

Specifically, L¹ to L³ in Chemical Formula 1 may each independently be a single bond, a substituted or unsubstituted phenylene group, or a substituted or unsubstituted biphenylene group.

Specifically, Ar¹ and Ar² of Chemical Formula 1 may each independently be a substituted or unsubstituted phenyl group, a substituted or unsubstituted biphenyl group, a substituted or unsubstituted terphenyl group, a substituted or unsubstituted naphthyl group, a substituted or unsubstituted carbazolyl group, a substituted or unsubstituted indolocarbazolyl group, a substituted or unsubstituted dibenzofuranyl group, a substituted or unsubstituted dibenzothiophenyl group, a substituted or unsubstituted fused carbazolyl group, a substituted or unsubstituted fused dibenzofuranyl group, a substituted or unsubstituted fused dibenzothiophenyl group, a substituted or unsubstituted fused indolocarbazolyl group, a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted quinoxalinyl group, a substituted or unsubstituted quinazolinyl group, or a substituted or unsubstituted benzoquinazolinyl group.

For example, Ar¹ and Ar² in Chemical Formula 1 may each independently be a substituted or unsubstituted phenyl group, a substituted or unsubstituted biphenyl group, a substituted or unsubstituted terphenyl group, a substituted or unsubstituted fluorenyl group, a substituted or unsubstituted dibenzofuranyl group, or a substituted or unsubstituted dibenzothiophenyl group.

Specifically, R¹ to R³ in Chemical Formula 1 may each independently be hydrogen, deuterium, a substituted or unsubstituted C1 to C10 alkyl group, or a substituted or unsubstituted C6 to C12 aryl group.

For example, R¹ to R³ in Chemical Formula 1 may each independently be hydrogen, deuterium, a substituted or unsubstituted C1 to C5 alkyl group, a substituted or unsubstituted phenyl group, or a substituted or unsubstituted biphenyl group.

For example, R¹ to R³ may be linked to adjacent groups to form a substituted or unsubstituted heteroaromatic polycyclic ring, for example, a substituted or unsubstituted benzofuranpyrimidine, or a substituted or unsubstituted benzothiophenpyrimidine.

For example, Chemical Formula 1 may be represented by any one of Chemical Formula 1-4 to Chemical Formula 1-6.

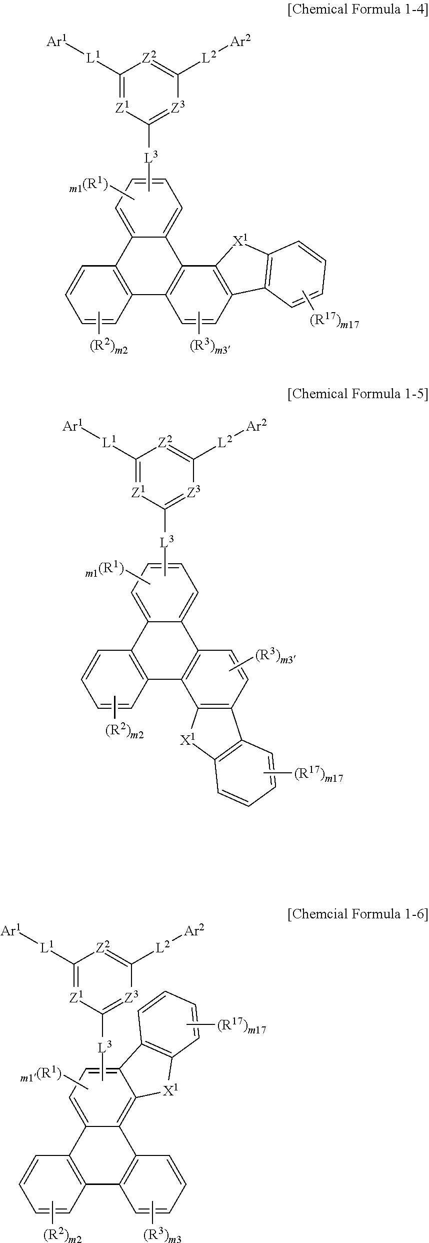

In Chemical Formula 1-4 to Chemical Formula 1-6, the definitions of Z¹ to Z³, R¹ to R³, L¹ to L³, Ar¹, Ar², and m1 to m3 are the same as described above,
X¹ is O or S,
R¹⁷s are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
m17 is one of integers of 1 to 4,
m1′ is 1, and
m3′ is an integer of 1 or 2.

When m3′ is 2 or more, each R³ may be the same or different.

When m17 is 2 or more, each R¹⁷ may be the same or different.

In an embodiment, each of R¹⁷s may each independently be hydrogen, deuterium, a substituted or unsubstituted C1 to C10 alkyl group, or a substituted or unsubstituted C6 to C12 aryl group.

Chemical Formula 1 may be represented by, for example, Chemical Formula 1-A or Chemical Formula 1-B, depending on the substitution position of the 6-membered ring including Z¹ to Z³.

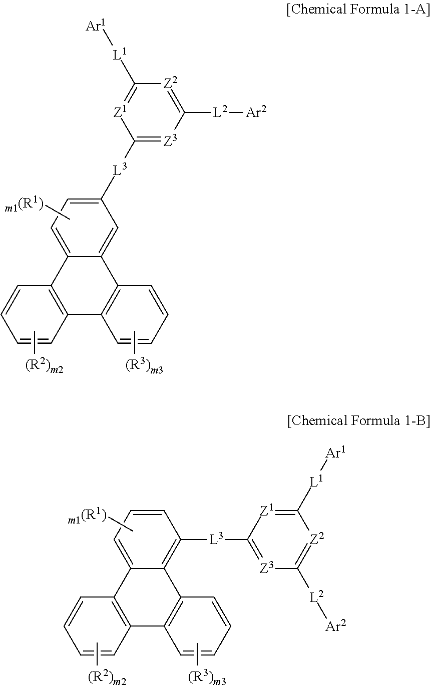

In Chemical Formula 1-A and Chemical Formula 1-B, the definitions of Z¹ to Z³, L¹ to L³, R¹ to R³, L¹ to L³, Ar¹, Ar², and m1 to m3 are the same as described above.

In a specific embodiment of the present invention, the first compound may be represented by Chemical Formula 1-A.

The first compound may be, for example, one selected from the compounds listed in Group 1.

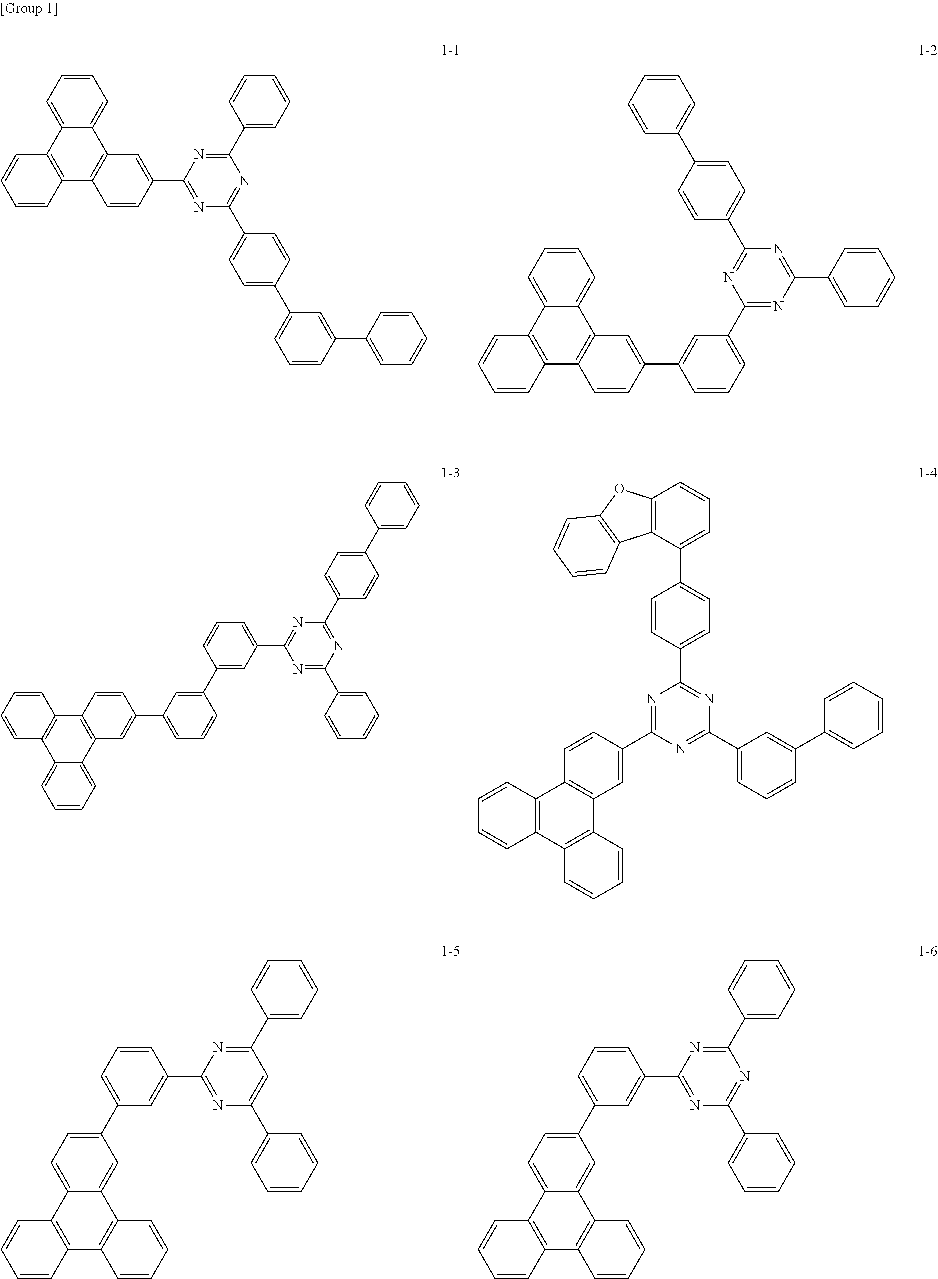

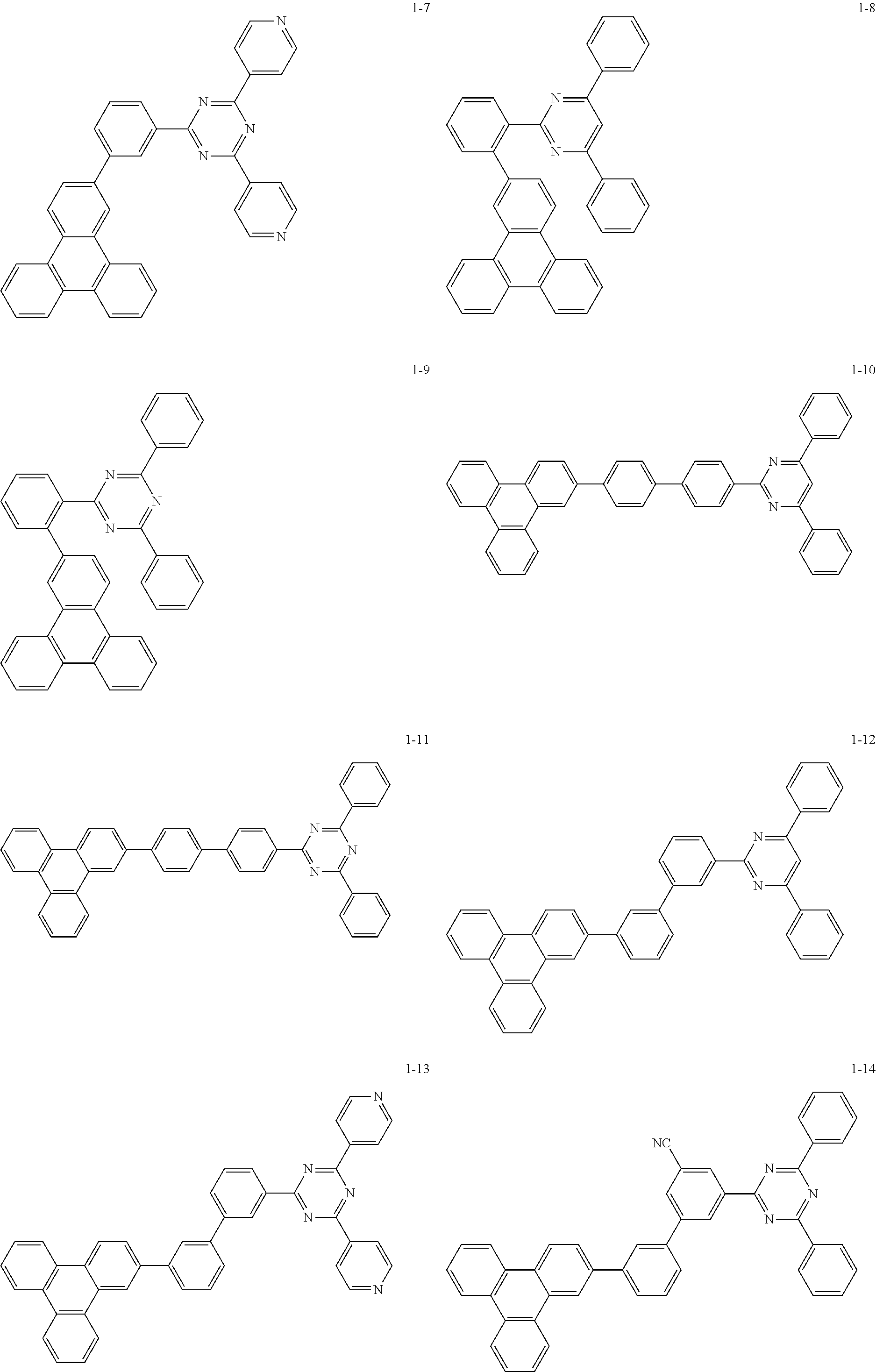

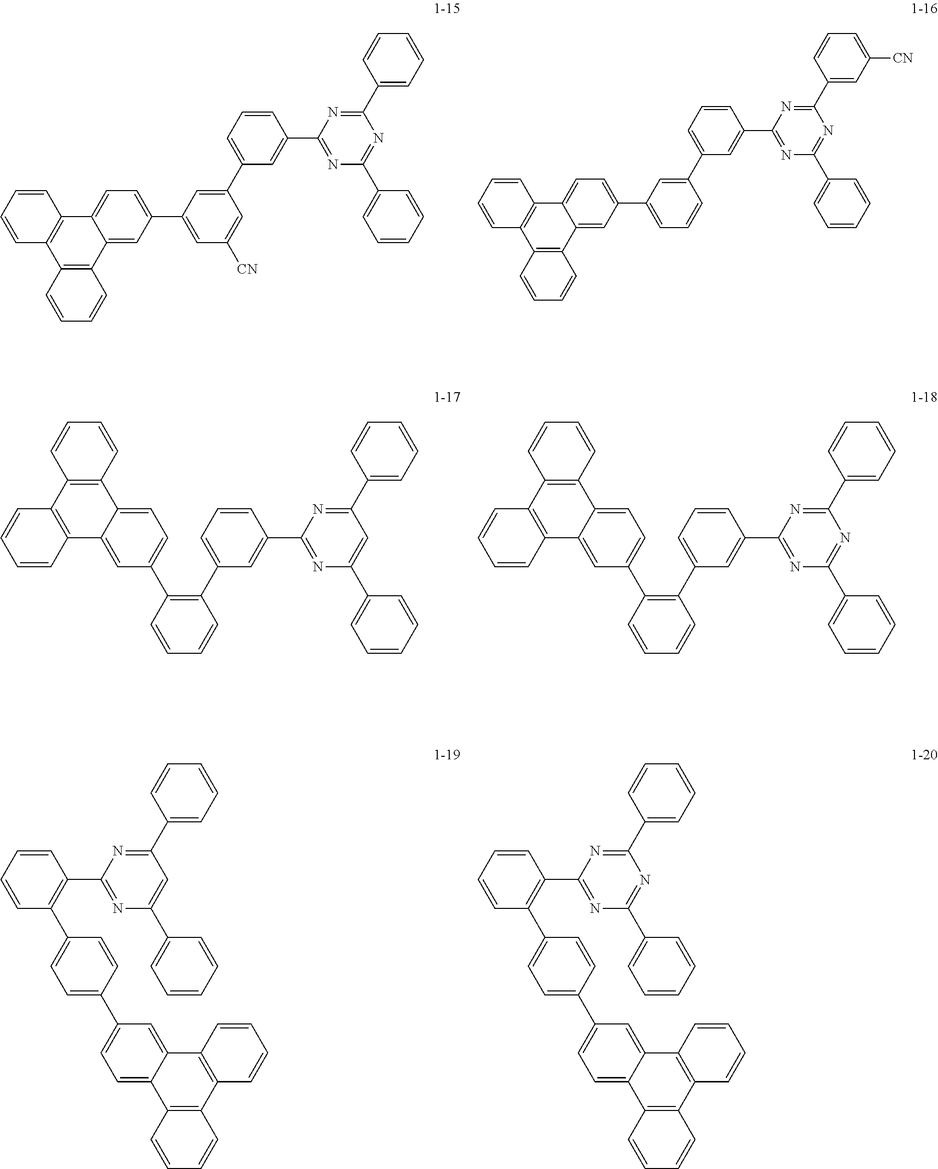 [SMILES: N#Cc1cc(-c2cccc(-c3nc(-c4ccccc4)nc(-c4ccccc4)n3)c2)cc(-c2ccc3c4ccccc4c4ccccc4c3c2)c1 ; N#Cc1cccc(-c2nc(-c3ccccc3)nc(-c3cccc(-c4cccc(-c5ccc6c7ccccc7c7ccccc7c6c5)c4)c3)n2)c1 ; c1ccc(-c2cc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1]

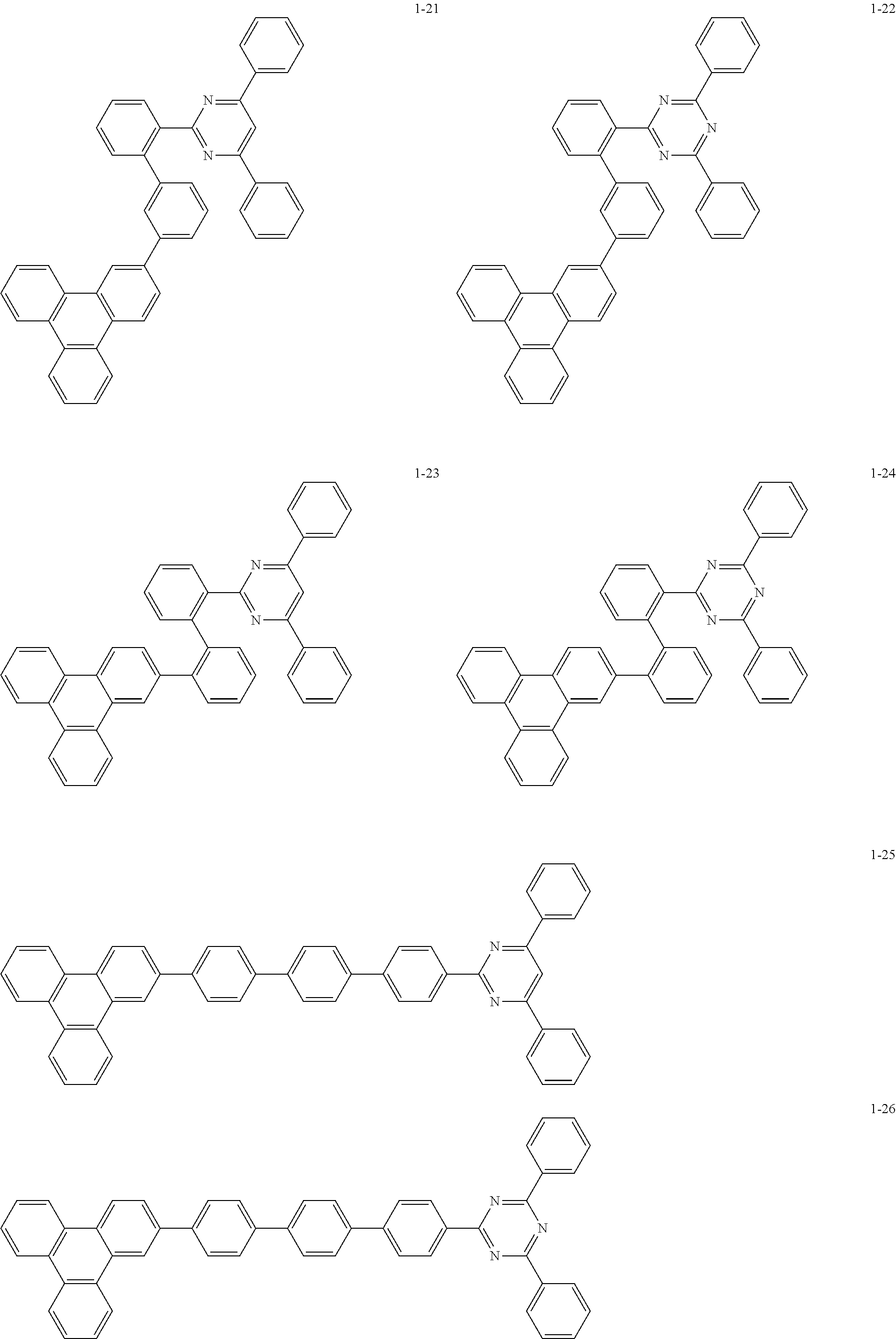

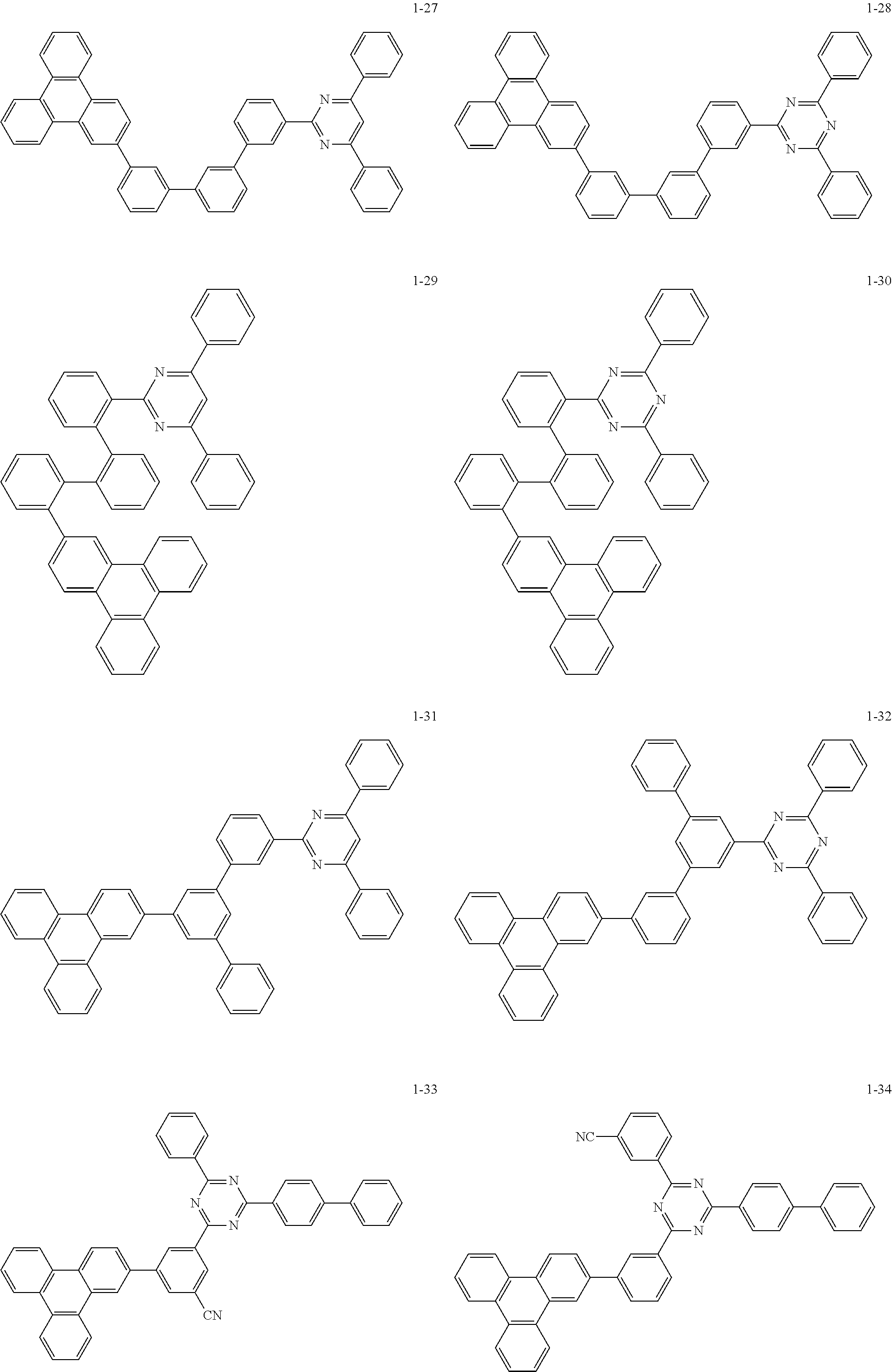

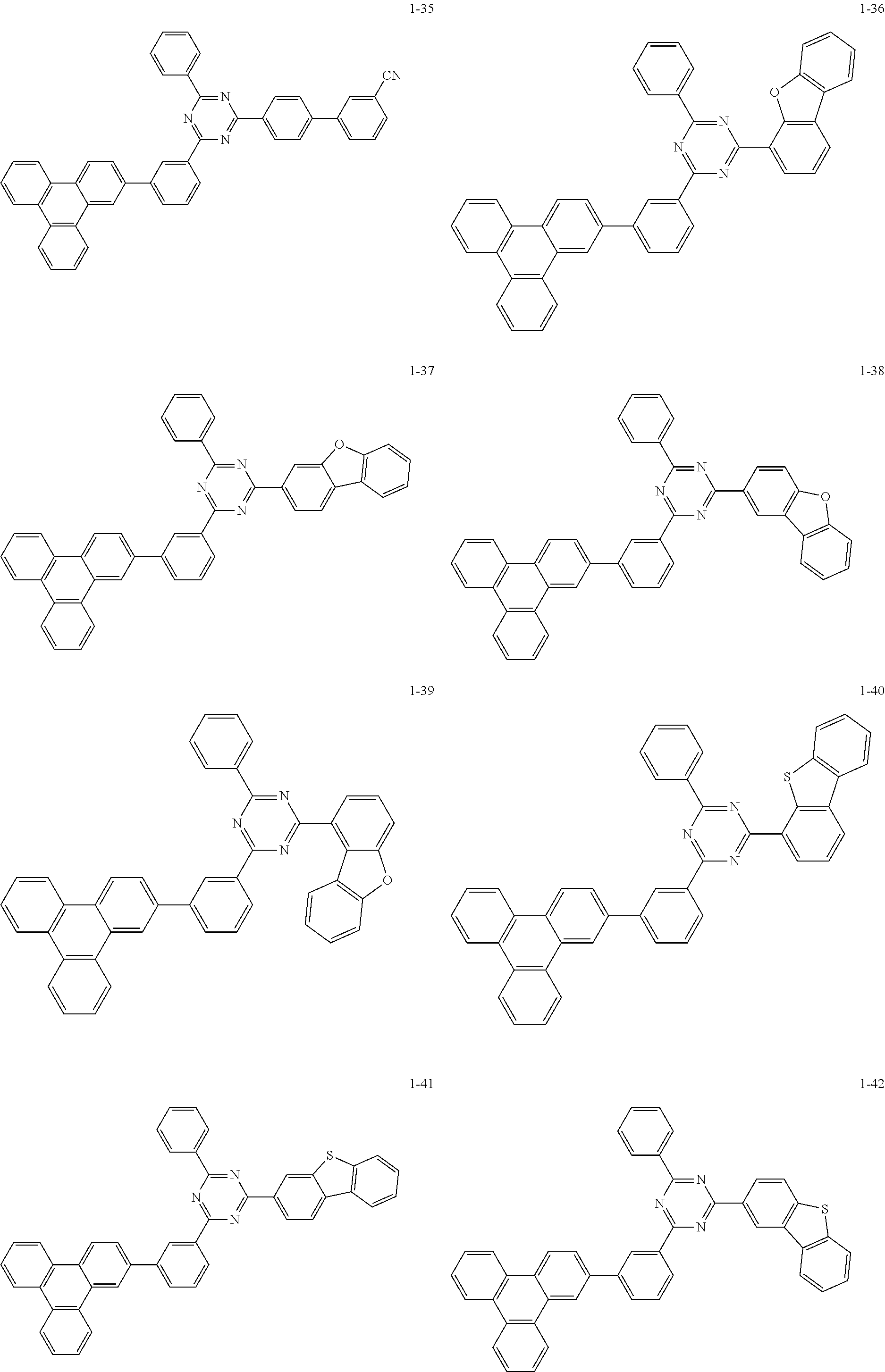

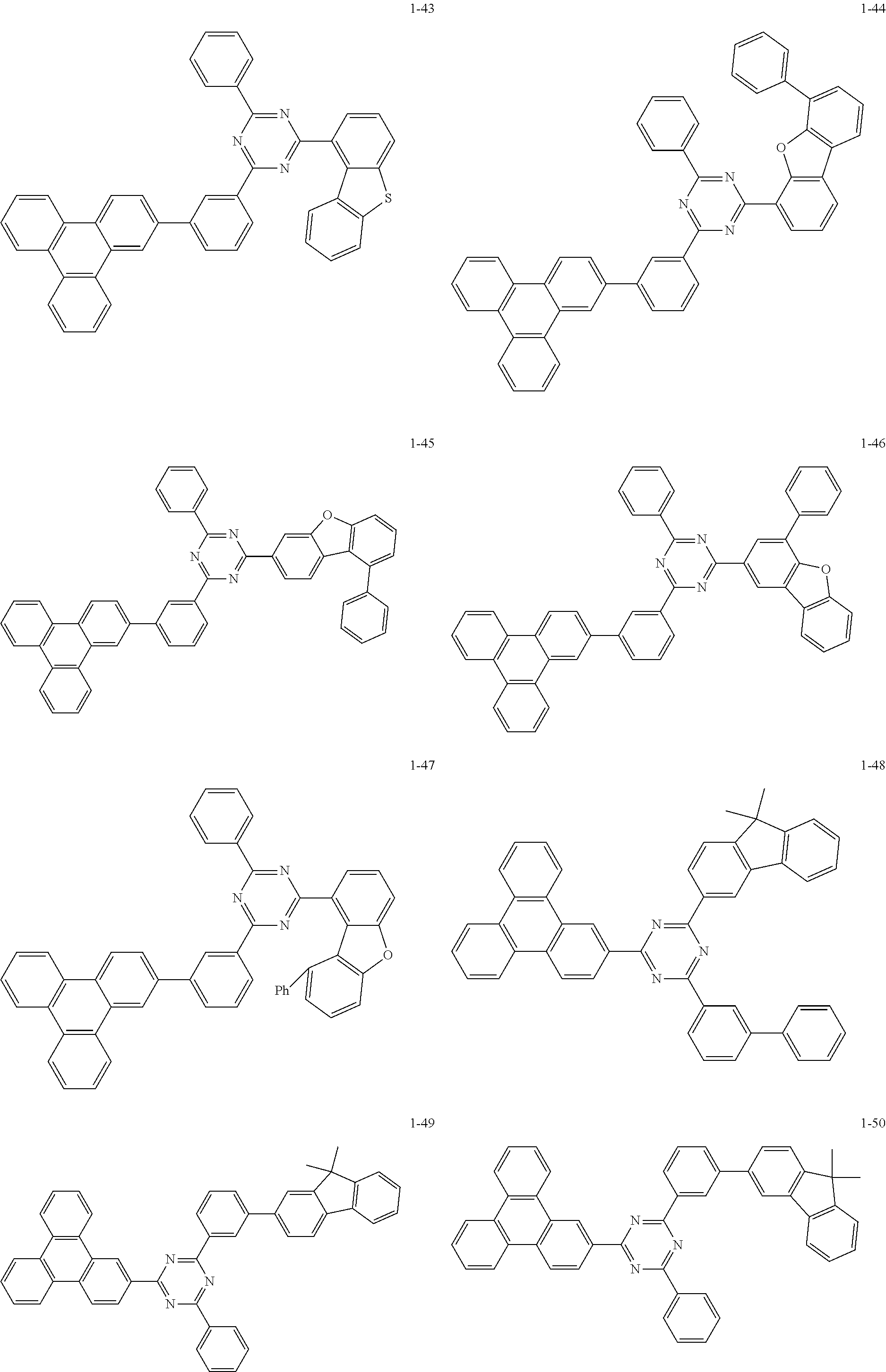

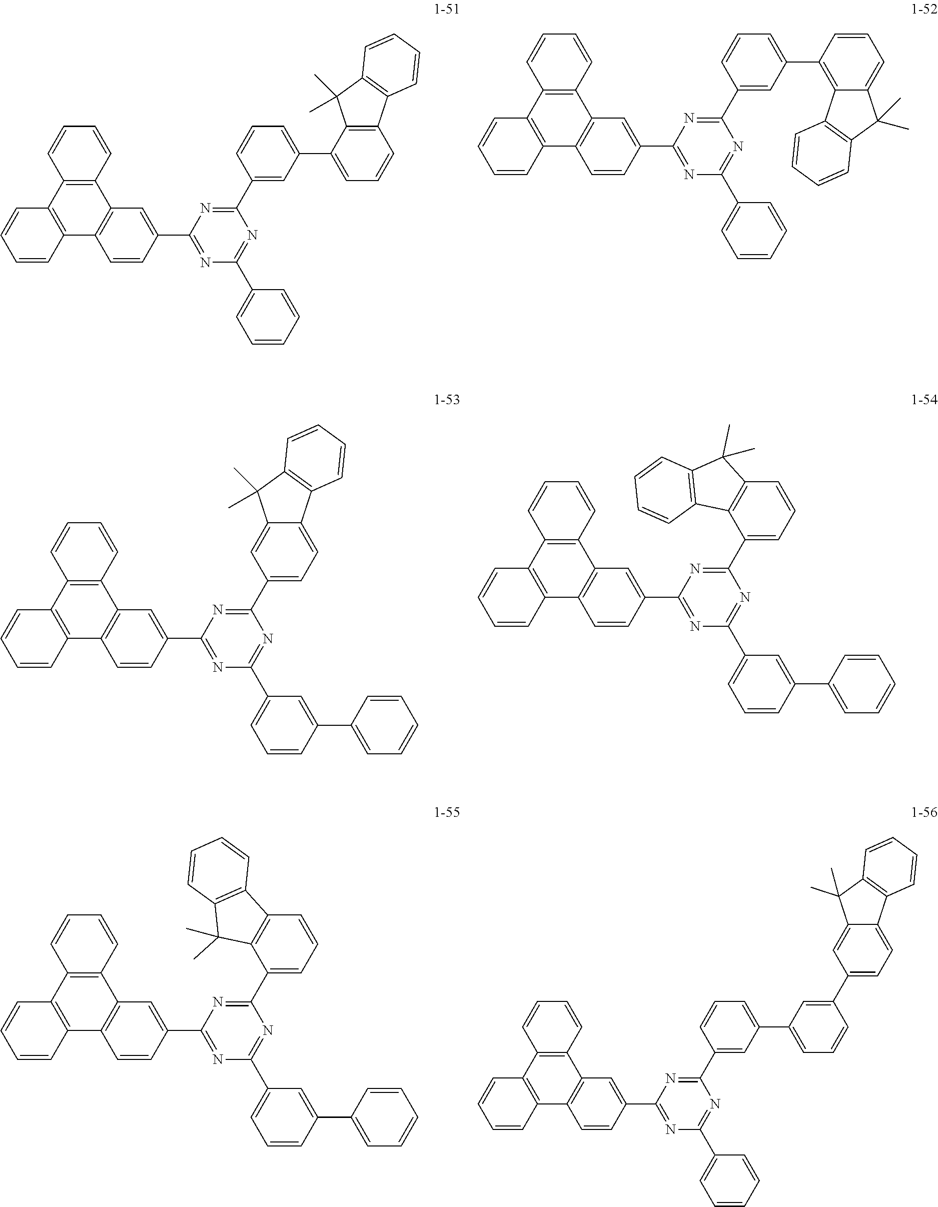

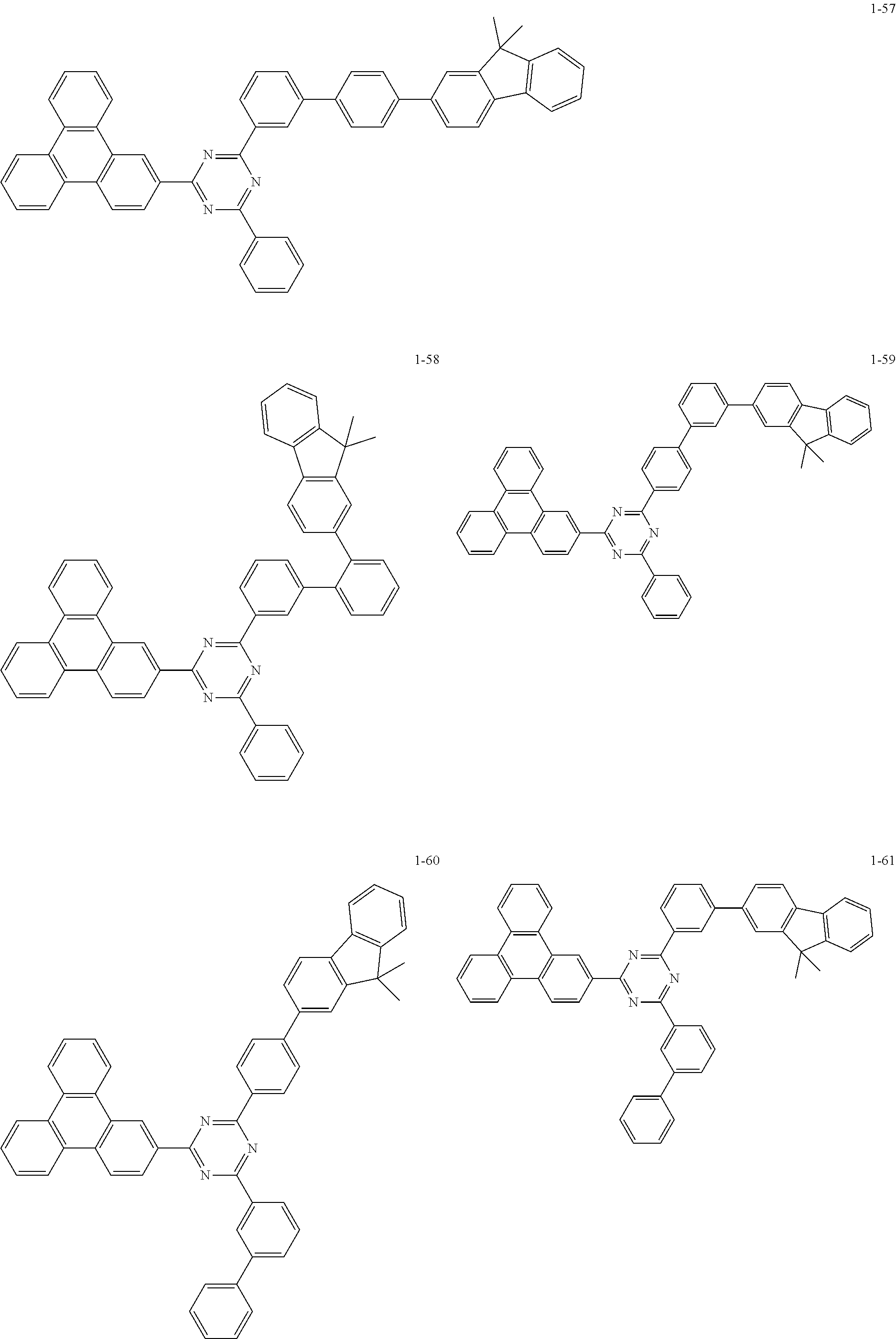 [SMILES: CC1(C)c2ccccc2-c2ccc(-c3ccc(-c4cccc(-c5nc(-c6ccccc6)nc(-c6ccc7c8ccccc8c8ccccc8c7c6)n5)c4)cc3)cc21 ; CC1(C)c2ccccc2-c2ccc(-c3ccccc3-c3cccc(-c4nc(-c5ccccc5)nc(-c5ccc6c7ccccc7c7ccccc7c6c5)n4)c3)cc21 ; CC1(C)c2ccccc2-c2ccc(-c3cccc(-c4ccc(-c5nc(-c6ccccc6)nc(-c6ccc7c8ccccc8c8ccccc8c7c6)n5)cc4)c3)cc21 ; CC1(C)c2ccccc2-c2ccc(-c3ccc(-c4nc(-c5cccc(-c6ccccc6)c5)nc(-c5ccc6c7ccccc7c7ccccc7c6c5)n4)cc3)cc21 ; CC1(C)c2ccccc2-c2ccc(-c3cccc(-c4nc(-c5cccc(-c6ccccc6)c5)nc(-c5ccc6c7ccccc7c7ccccc7c6c5)n4)c3)cc21]

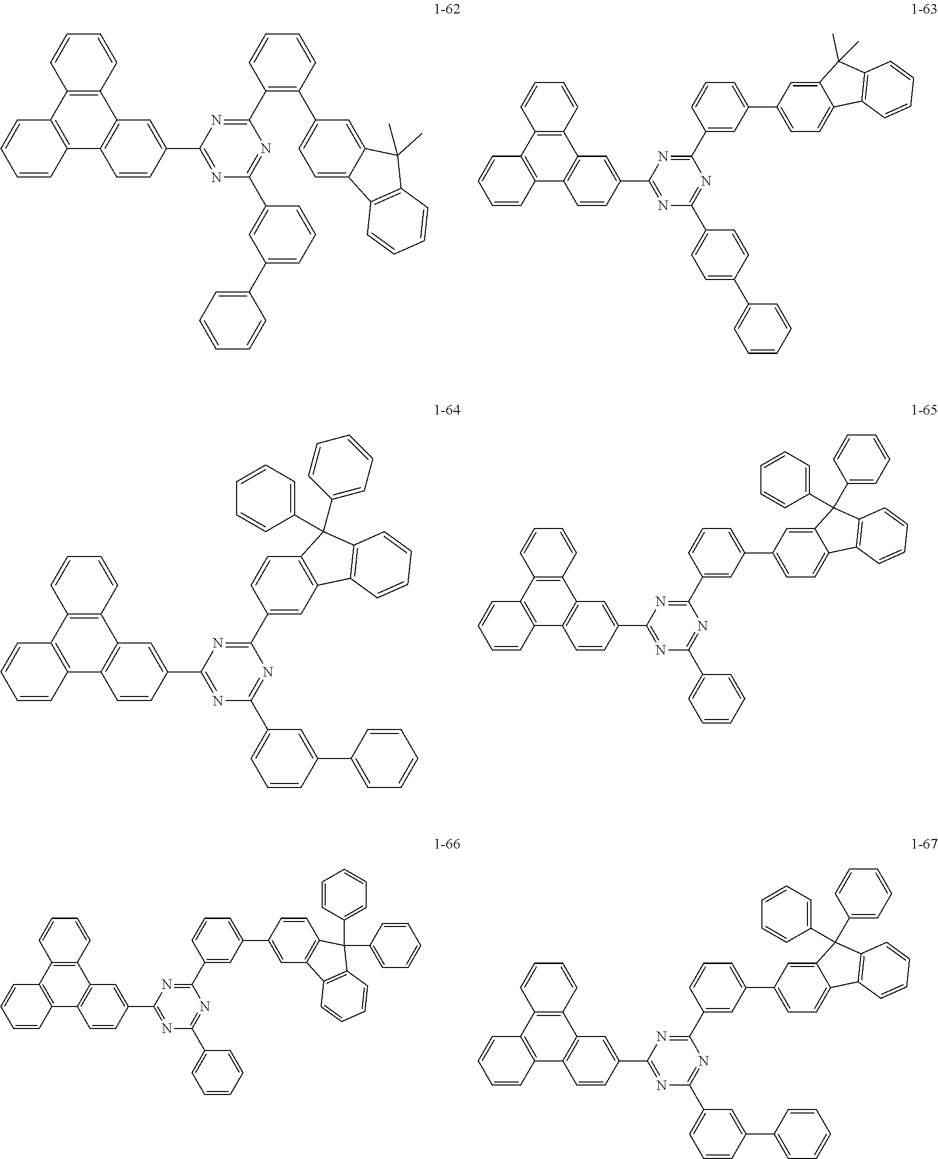

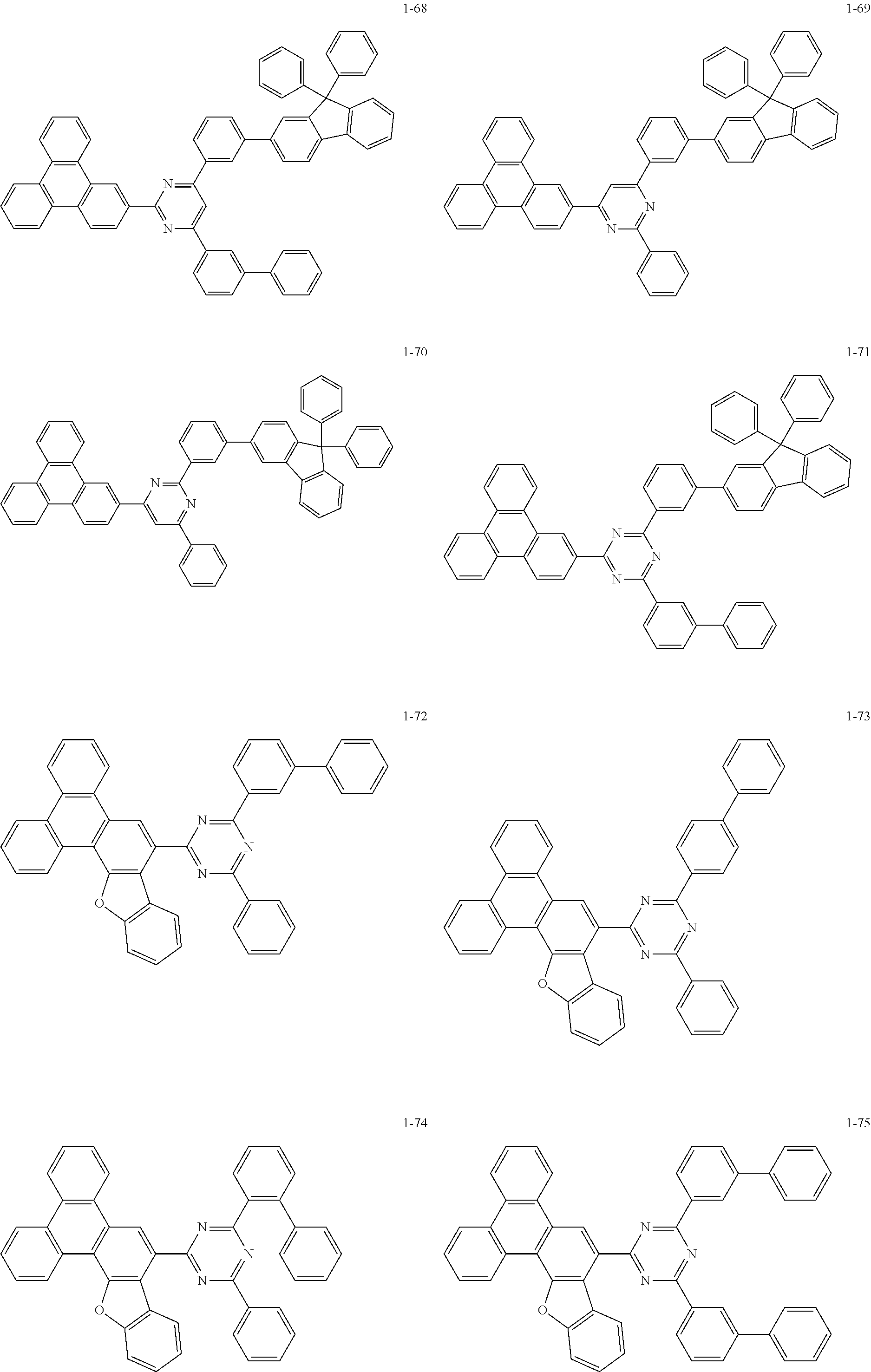

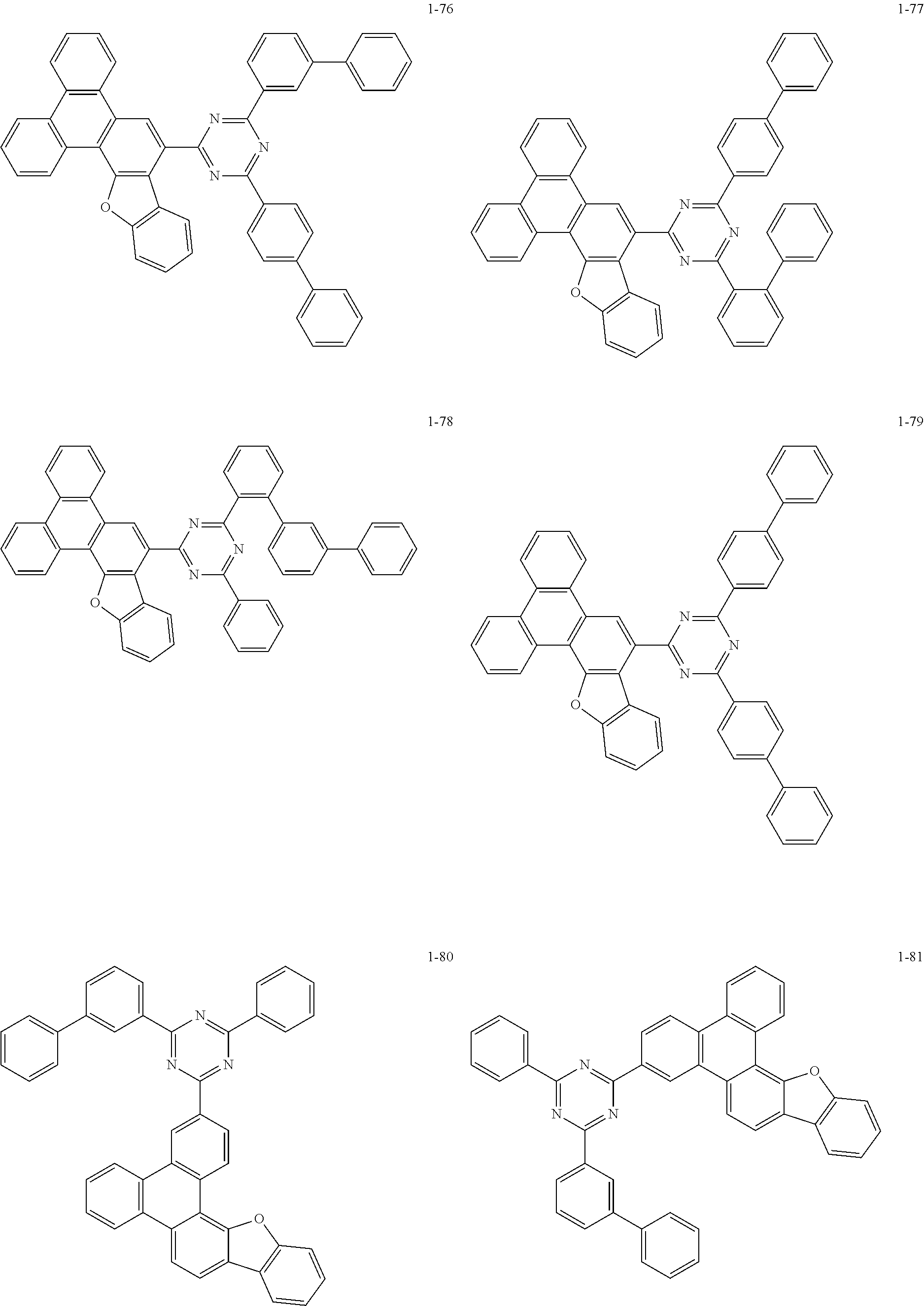

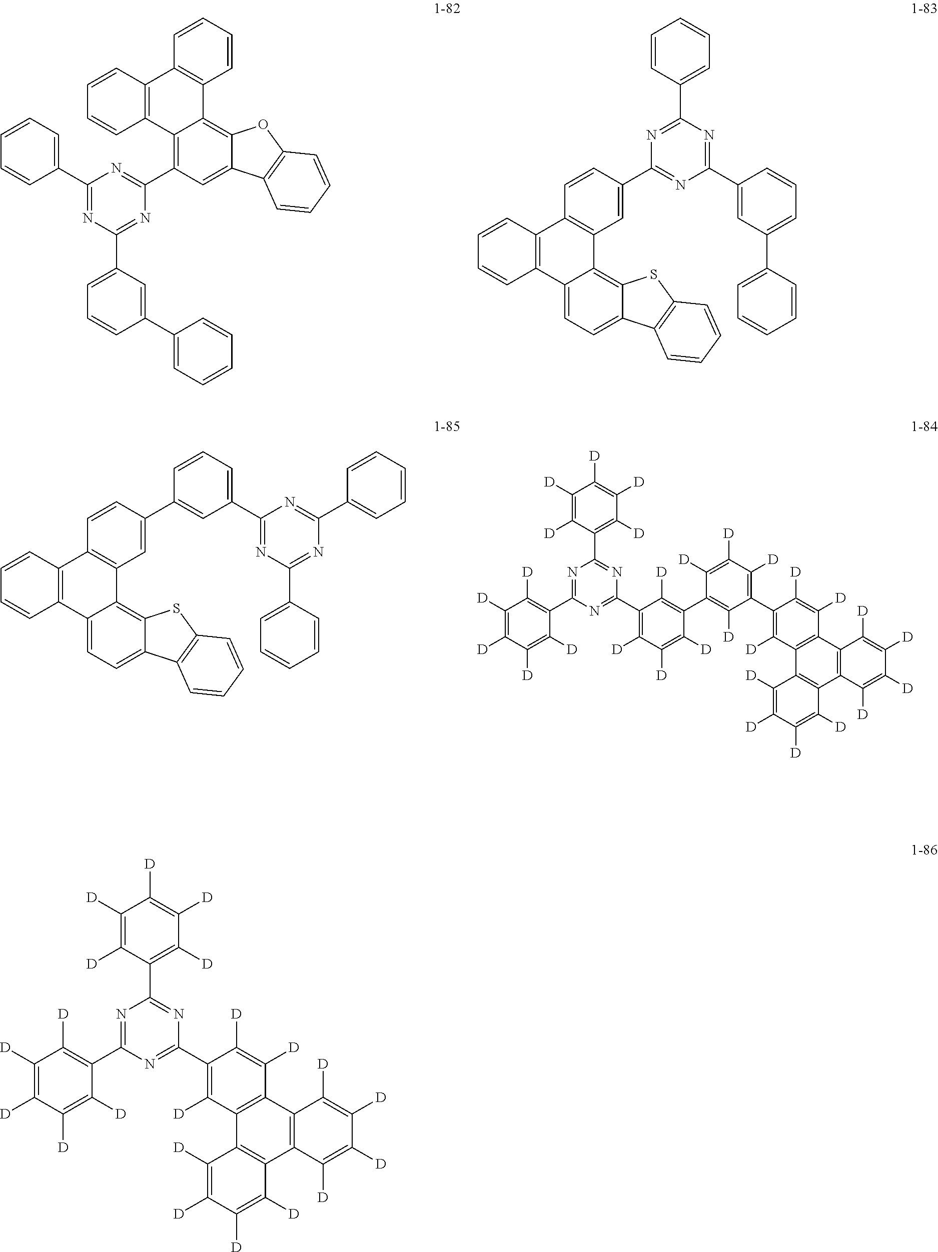

The second compound having the hole characteristics has a structure in which carbazole or indolocarbazole is substituted with a substituted or unsubstituted C6 to C30 aryl group, or a substituted or unsubstituted C2 to C30 heterocyclic group and is for example represented by Chemical Formula 2 or a combination of Chemical Formula 3 and Chemical Formula 4.

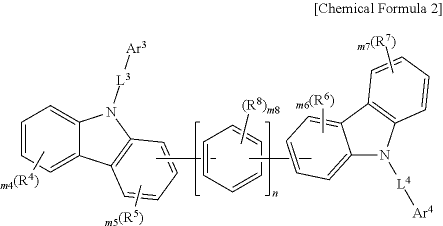

In Chemical Formula 2,
Ar³ and Ar⁴ are each independently a substituted or unsubstituted C6 to C30 aryl group or a substituted or unsubstituted C2 to C30 heterocyclic group,
L³ and L⁴ are each independently a single bond or a substituted or unsubstituted C6 to C20 arylene group,
R⁴ to R⁸ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
m5 and m6 are each independently one of integers of 1 to 3,
m4, m7, and m8 are each independently one of integers of 1 to 4, and
n is one of integers of 0 to 2;

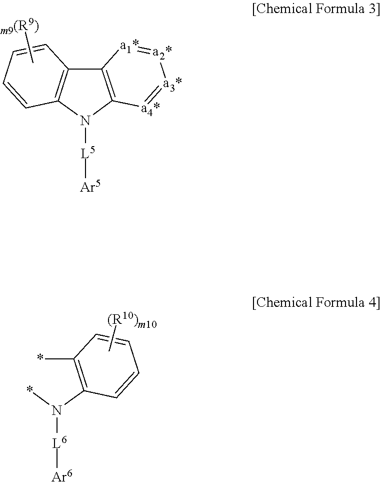

wherein, in Chemical Formulas 3 and 4,
Ar⁵ and Ar⁶ are each independently a substituted or unsubstituted C6 to C30 aryl group or a substituted or unsubstituted C2 to C30 heterocyclic group,
a₁* to a₄* in Chemical Formula 3 are each independently a linking carbon (C) or C-L^{b}-R^{b},
among a₁* to a₄* in Chemical Formula 3, two adjacent ones are linked to * in Chemical Formula 4,
L^{b}, L⁵, and L⁶ are each independently a single bond or a substituted or unsubstituted C6 to C20 arylene group,
R^{b}, R⁹, and R¹⁰ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group, and
m9 and m10 are each independently one of integers of 1 to 4.

When m4 is 2 or more, each R⁴ may be the same or different.

When m5 is 2 or more, each R⁵ may be the same or different.

When m6 is 2 or more, each R6 may be the same or different.

When m7 is 2 or more, each R⁶ may be the same or different.

When m8 is 2 or more, each R⁸ may be the same or different.

When m9 is 2 or more, each R⁹ may be the same or different.

When m10 is 2 or more, each R¹⁰ may be the same or different.

For example, in Chemical Formula 2, Ar³ and Ar⁴ may each independently be a substituted or unsubstituted C6 to C20 aryl group or a substituted or unsubstituted C2 to C20 heterocyclic group.

For example, in Chemical Formula 2, Ar³ and Ar⁴ may each independently be a substituted or unsubstituted C6 to C20 aryl group, or a substituted or unsubstituted C2 to C20 heterocyclic group, and at least one of Ar³ and Ar⁴ may be a C6 to C20 aryl group substituted or unsubstituted with one or more deuteriums, or a C2 to C20 heterocyclic group substituted or unsubstituted with one or more deuteriums.

For example, in Chemical Formula 2, R⁴ to R⁸ may each independently be hydrogen, deuterium, a cyano group, a halogen, a substituted or unsubstituted amine group, a substituted or unsubstituted C1 to C10 alkyl group, a substituted or unsubstituted C6 to C20 aryl group, or a substituted or unsubstituted C2 to C20 heterocyclic group.

For example, in Chemical Formula 2, R⁴ to R⁸ may each independently be hydrogen, deuterium, a cyano group, a halogen, a substituted or unsubstituted amine group, a substituted or unsubstituted C1 to C10 alkyl group, a substituted or unsubstituted C6 to C20 aryl group or a substituted or unsubstituted C2 to C20 heterocyclic group, and at least one of R⁴ to R⁸ may be deuterium, a C1 to C10 alkyl group substituted or unsubstituted with one or more deuteriums, a C6 to C20 aryl group substituted or unsubstituted with one or more deuteriums, or a C2 to C20 heterocyclic group substituted or unsubstituted with one or more deuteriums.

For example, in Chemical Formula 2, at least one of Ar³ and Ar⁴ may be a C6 to C20 aryl group substituted or unsubstituted with one or more deuteriums, or a C2 to C20 heterocyclic group substituted or unsubstituted with one or more deuteriums, and at least one of R⁴ to R⁸ may be deuterium, a C1 to C10 alkyl group substituted or unsubstituted with one or more deuteriums, a C6 to C20 aryl group substituted or unsubstituted with one or more deuteriums, or a C2 to C20 heterocyclic group substituted or unsubstituted with one or more deuteriums.

As a specific example, in Chemical Formula 2, Ar³ and Ar⁴ may each independently be a substituted or unsubstituted C6 to C20 aryl group, a substituted or unsubstituted dibenzofuranyl group, a substituted or unsubstituted dibenzothiophenyl group, or a substituted or unsubstituted carbazolyl group.

As a specific example, in Chemical Formula 2, Ar³ and Ar⁴ may each independently be a substituted or unsubstituted C6 to C20 aryl group, a substituted or unsubstituted dibenzofuranyl group, a substituted or unsubstituted dibenzothiophenyl group, or a substituted or unsubstituted carbazolyl group, and at least one of Ar³ and Ar⁴ may be a C6 to C20 aryl group substituted or unsubstituted with one or more deuteriums, a dibenzofuranyl group substituted or unsubstituted with one or more deuteriums, a dibenzothiophenyl group substituted or unsubstituted with one or more deuteriums, or a carbazolyl group substituted or unsubstituted with one or more deuteriums.

As a specific example, in Chemical Formula 2, at least one of Ar³ and Ar⁴ may be a C6 to C20 aryl group substituted or unsubstituted with one or more deuteriums, a dibenzofuranyl group substituted or unsubstituted with one or more deuteriums, a dibenzothiophenyl group substituted or unsubstituted with one or more deuteriums, or a carbazolyl group substituted or unsubstituted with one or more deuteriums, and at least one of R⁴ to R⁸ may be deuterium, a C1 to C10 alkyl group substituted or unsubstituted with one or more deuteriums, a C6 to C20 aryl group substituted or unsubstituted with one or more deuteriums, or a C2 to C20 heterocyclic group substituted or unsubstituted with one or more deuteriums.

For example, in Chemical Formula 3 and Chemical Formula 4, Ar⁵ and Ar⁶ may each independently be a substituted or unsubstituted C6 to C20 aryl group, or a substituted or unsubstituted C2 to C20 heterocyclic group.

For example, in Chemical Formula 3 and Chemical Formula 4, Ar⁵ and Ar⁶ may each independently be a substituted or unsubstituted C6 to C20 aryl group, or a substituted or unsubstituted C2 to C20 heterocyclic group, and at least one of Ar⁵ and Ar⁶ may be a C6 to C20 aryl group substituted or unsubstituted with one or more deuteriums, or a C2 to C20 heterocyclic group substituted or unsubstituted with one or more deuteriums.

For example, in Chemical Formula 3 and Chemical Formula 4, R^{b}, R⁹, and R¹⁰ may each independently be hydrogen, deuterium, a cyano group, a halogen, a substituted or unsubstituted amine group, a substituted or unsubstituted C1 to C10 alkyl group, a substituted or unsubstituted C6 to C20 aryl group or a substituted or unsubstituted C2 to C20 heterocyclic group.

For example, in Chemical Formula 3 and Chemical Formula 4, R^{b}, R⁹, and R¹⁰ may each independently be hydrogen, deuterium, a cyano group, a halogen, a substituted or unsubstituted amine group, a substituted or unsubstituted C1 to C10 alkyl group, a substituted or unsubstituted C6 to C20 aryl group or a substituted or unsubstituted C2 to C20 heterocyclic group, and at least one of R^{b}, R⁹, and R¹⁰ may be deuterium, a C1 to C10 alkyl group substituted or unsubstituted with one or more deuteriums, a C6 to C20 aryl group substituted or unsubstituted with one or more deuteriums, or a C2 to C20 heterocyclic group substituted or unsubstituted with one or more deuteriums.

For example, in Chemical Formula 3 and Chemical Formula 4, at least one of Ar⁵ and Ar⁶ may be a C6 to C20 aryl group substituted or unsubstituted with one or more deuteriums, or a C2 to C20 heterocyclic group substituted or unsubstituted with one or more deuteriums, and at least one of R^{b}, R⁹, and R¹⁰ may be deuterium, a C1 to C10 alkyl group substituted or unsubstituted with one or more deuteriums, a C6 to C20 aryl group substituted or unsubstituted with one or more deuteriums, or a C2 to C20 heterocyclic group substituted or unsubstituted with one or more deuteriums.

As a specific example, in Chemical Formula 3 and Chemical Formula 4, Ar⁵ and Ar⁶ may each independently be a substituted or unsubstituted C6 to C20 aryl group, a substituted or unsubstituted dibenzofuranyl group, a substituted or unsubstituted dibenzothiophenyl group, or a substituted or unsubstituted carbazolyl group.

As a specific example, in Chemical Formula 3 and Chemical Formula 4, Ar⁵ and Ar⁶ may each independently be a substituted or unsubstituted C6 to C20 aryl group, a substituted or unsubstituted dibenzofuranyl group, a substituted or unsubstituted dibenzothiophenyl group, or a substituted or unsubstituted carbazolyl group, and at least one of Ar⁵ and Ar⁶ may be a C6 to C20 aryl group substituted or unsubstituted with one or more deuteriums, a dibenzofuranyl group substituted or unsubstituted with one or more deuteriums, a dibenzothiophenyl group substituted or unsubstituted with one or more deuteriums, or a carbazolyl group substituted or unsubstituted with one or more deuteriums.

As a specific example, in Chemical Formula 3 and Chemical Formula 4, at least one of Ar⁵ and Ar⁶ may be a C6 to C20 aryl group substituted or unsubstituted with one or more deuteriums, a dibenzofuranyl group substituted or unsubstituted with one or more deuteriums, a dibenzothiophenyl group substituted or unsubstituted with one or more deuteriums, or a carbazolyl group substituted or unsubstituted with one or more deuteriums, and at least one of R^{b}, R⁹, and R¹⁰ may be deuterium, a C1 to C10 alkyl group substituted or unsubstituted with one or more deuteriums, a C6 to C20 aryl group substituted or unsubstituted with one or more deuteriums, or a C2 to C20 heterocyclic group substituted or unsubstituted with one or more deuteriums.

As a more specific example, in Chemical Formula 2, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted carbazolyl group, a substituted or unsubstituted dibenzothiophenyl group, a substituted or unsubstituted dibenzofuranyl group, or a substituted or unsubstituted fluorenyl group,
in Chemical Formula 2, L³ and L⁴ may each independently be a single bond, a substituted or unsubstituted phenylene group, a substituted or unsubstituted biphenylene group, or a substituted or unsubstituted naphthalenylene group,
in Chemical Formula 2, R⁴ to R⁸ may each independently be hydrogen, deuterium, a substituted or unsubstituted C6 to C12 aryl group, a substituted or unsubstituted carbazolyl group, a substituted or unsubstituted dibenzofuranyl group or a substituted or unsubstituted dibenzothiophenyl group, and
n may be 0 or 1.

For example, in Chemical Formula 2, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted carbazolyl group, a substituted or unsubstituted dibenzothiophenyl group, a substituted or unsubstituted dibenzofuranyl group, or a substituted or unsubstituted fluorenyl group, and
at least one of Ar³ and Ar⁴ may be a phenyl group substituted or unsubstituted with one or more deuteriums, a biphenyl group substituted or unsubstituted with one or more deuteriums, a terphenyl group substituted or unsubstituted with one or more deuteriums, a naphthyl group substituted or unsubstituted with one or more deuteriums, an anthracenyl group substituted or unsubstituted with one or more deuteriums, a phenanthrenyl group substituted or unsubstituted with one or more deuteriums, a triphenylene group substituted or unsubstituted with one or more deuteriums, a carbazolyl group substituted or unsubstituted with one or more deuteriums, a dibenzothiophenyl group substituted or unsubstituted with one or more deuteriums, a dibenzofuranyl group substituted or unsubstituted with one or more deuteriums, or a fluorenyl group substituted or unsubstituted with one or more deuteriums.

For example, in Chemical Formula 2, R⁴ to R⁸ may each independently be hydrogen, deuterium, a substituted or unsubstituted C6 to C12 aryl group, a substituted or unsubstituted carbazolyl group, a substituted or unsubstituted dibenzofuranyl group, or a substituted or unsubstituted dibenzothiophenyl group, and
at least one of R⁴ to R⁸ may be a deuterium, a C6 to C12 aryl group substituted or unsubstituted with one or more deuteriums, a carbazolyl group substituted or unsubstituted with one or more deuteriums, a dibenzofuranyl group substituted or unsubstituted with one or more deuteriums, or a dibenzothiophenyl group substituted or unsubstituted with one or more deuteriums.

For example, in Chemical Formula 2, at least one of Ar³ and Ar⁴ may be a phenyl group substituted or unsubstituted with one or more deuteriums, a biphenyl group substituted or unsubstituted with one or more deuteriums, a terphenyl group substituted or unsubstituted with one or more deuteriums, a naphthyl group substituted or unsubstituted with one or more deuteriums, an anthracenyl group substituted or unsubstituted with one or more deuteriums, a phenanthrenyl group substituted or unsubstituted with one or more deuteriums, a triphenylene group substituted or unsubstituted with one or more deuteriums, a carbazolyl group substituted or unsubstituted with one or more deuteriums, a dibenzothiophenyl group substituted or unsubstituted with one or more deuteriums, a dibenzofuranyl group substituted or unsubstituted with one or more deuteriums, or a fluorenyl group substituted or unsubstituted with one or more deuteriums, and
in Chemical Formula 2, at least one of R⁴ to R⁸ may be deuterium, a C6 to C12 aryl group substituted or unsubstituted with one or more deuteriums, a carbazolyl group substituted or unsubstituted with one or more deuteriums, a dibenzofuranyl group substituted or unsubstituted with one or more deuteriums, or a dibenzothiophenyl group substituted or unsubstituted with one or more deuteriums.

As an example, in Chemical Formula 2, “substituted” refers to replacement of at least one hydrogen by deuterium, a C1 to C4 alkyl group, a C6 to C18 aryl group, or a C2 to C30 heteroaryl group.

In a specific embodiment of the present invention, Chemical Formula 2 may be represented by one of Chemical Formula 2-1 to Chemical Formula 2-15.

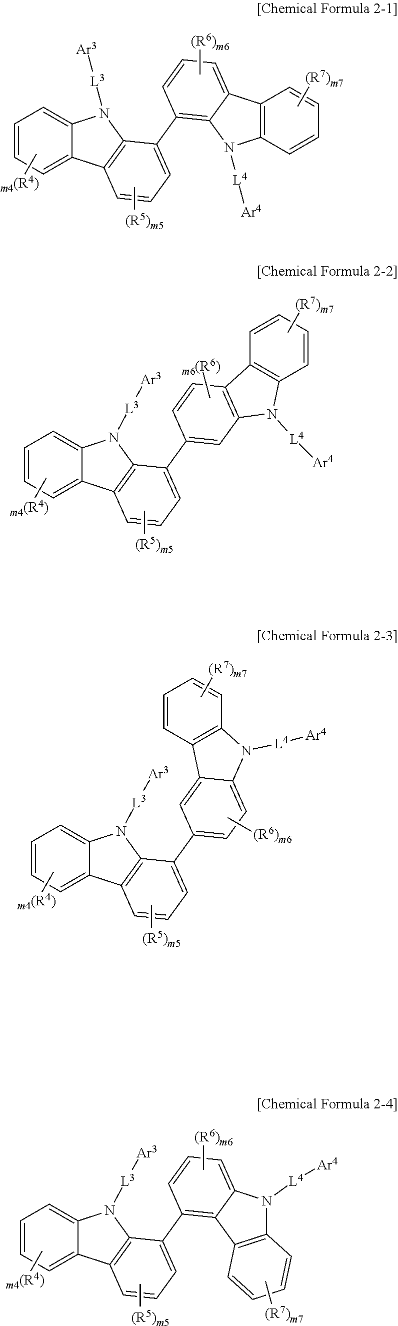

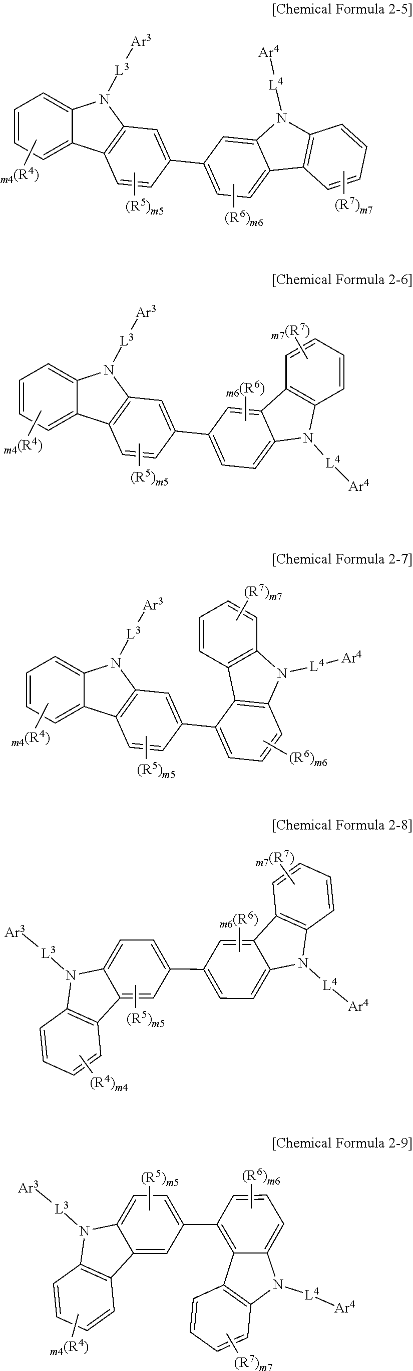

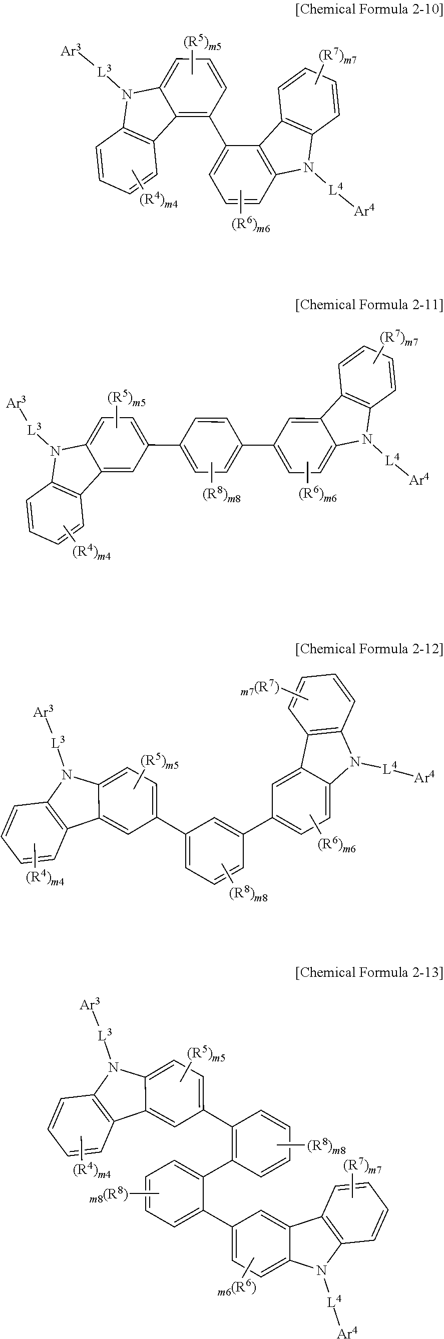

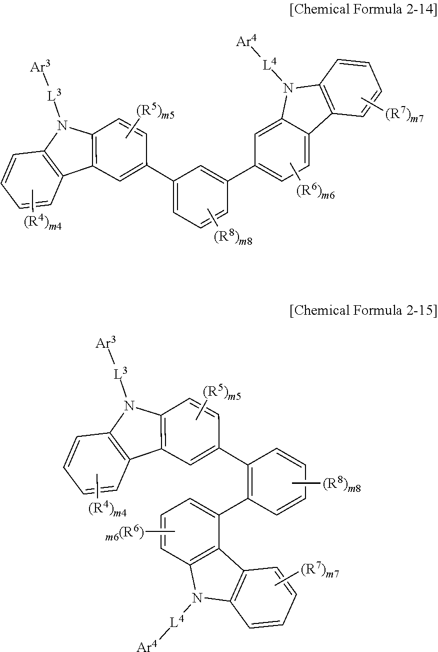

In Chemical Formula 2-1 to Chemical Formula 2-15, R⁴ to R⁸ may each independently be hydrogen, deuterium, a substituted or unsubstituted C6 to C12 aryl group, a substituted or unsubstituted carbazolyl group, a substituted or unsubstituted dibenzofuranyl group or a substituted or unsubstituted dibenzothiophenyl group, and L³-Ar³ and L⁴-Ar⁴ may each independently be one of the substituents listed in Group I.

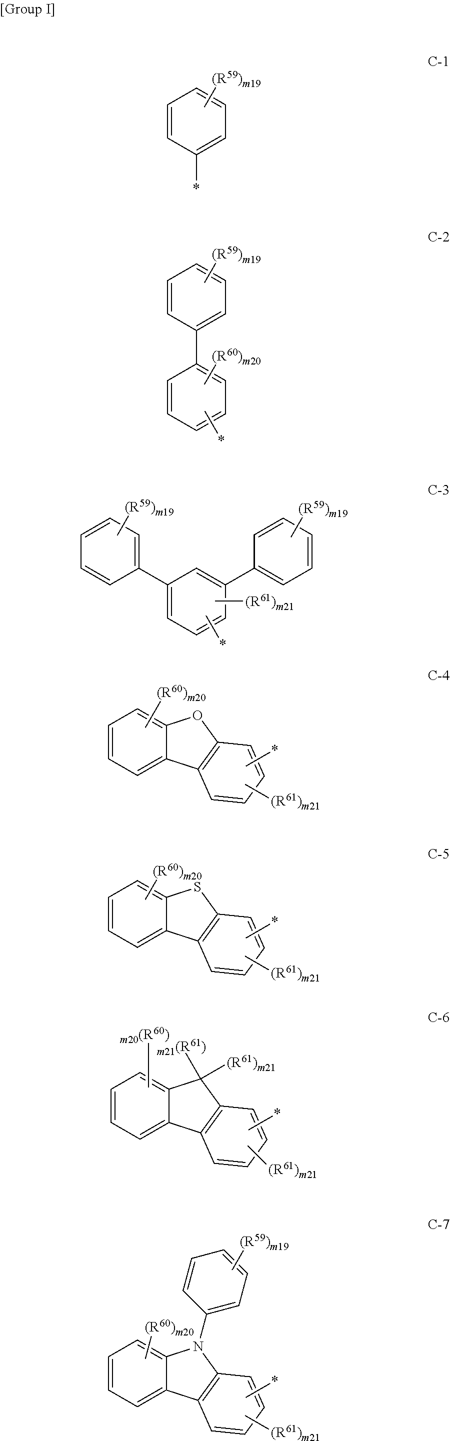

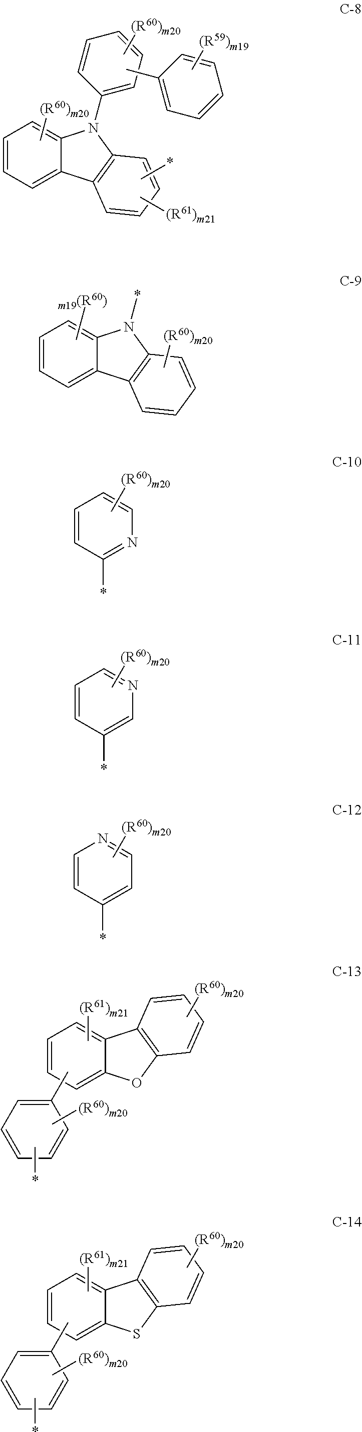

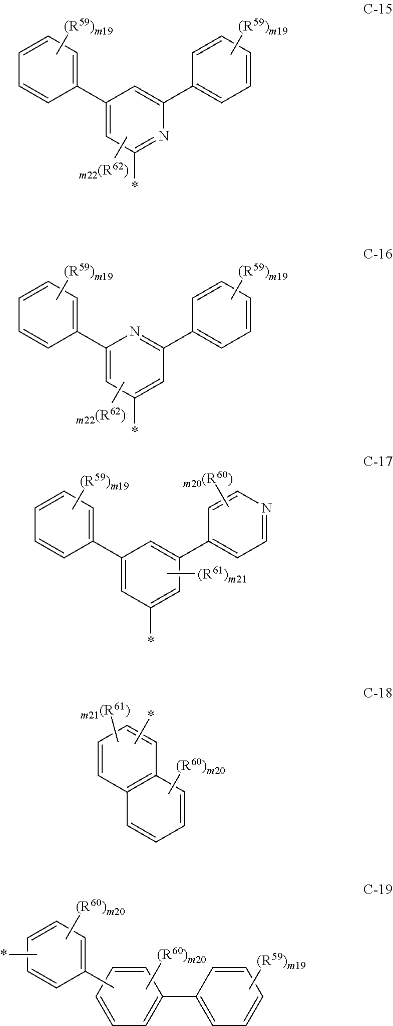

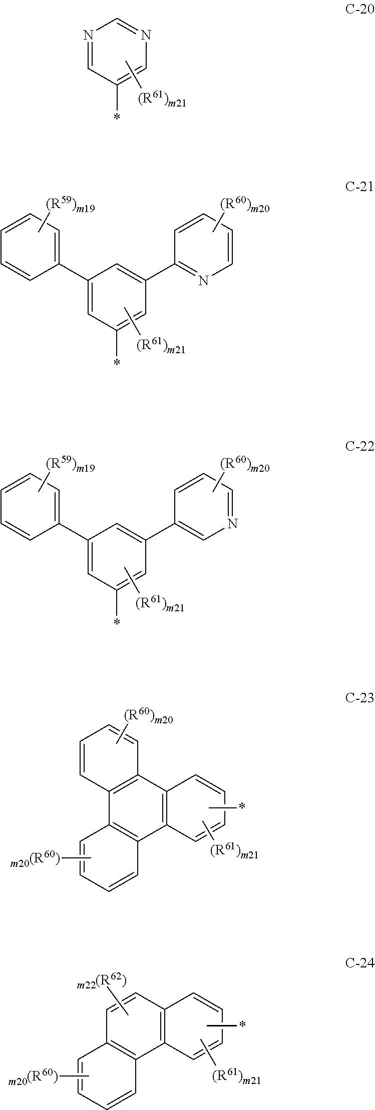

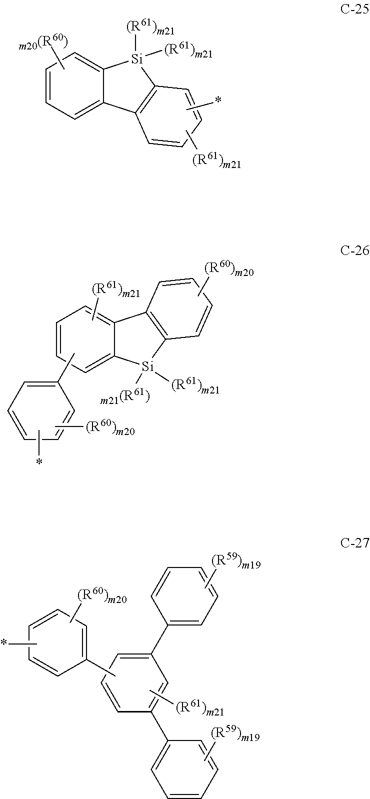

In Group I,
R⁵⁹ to R⁶² are each independently hydrogen, deuterium, C1 to C4 alkyl group, C6 to C18 aryl group, or C2 to C30 heteroaryl group,
m19 is one of integers of 1 to 5,
m20 is one of integers of 1 to 4,
m21 is one of integers of 1 to 3,
m12 is an integer of 1 or 2, and
* is a linking point.

When m19 is 2 or more, each R⁵⁹ may be the same or different.

When m20 is 2 or more, each R⁶¹ may be the same or different.

When m21 is 2 or more, each R⁶¹ may be the same or different.

When m22 is 2 or more, each R⁶² may be the same or different.

In an embodiment, Chemical Formula 2 may be represented by Chemical Formula 2-8.

In addition, in Chemical Formula 2-8, L³-Ar³ and L⁴-Ar⁴ may each independently be selected from Group I, and may be, for example, any one of C-1, C-2, C-3, C-4, C-7, C-8, and C-9.

For example, the second compound represented by the combination of Chemical Formula 3 and Chemical Formula 4 may be represented by any one of Chemical Formula 3A, Chemical Formula 3B, Chemical Formula 3C, Chemical Formula 3D, and Chemical Formula 3E.

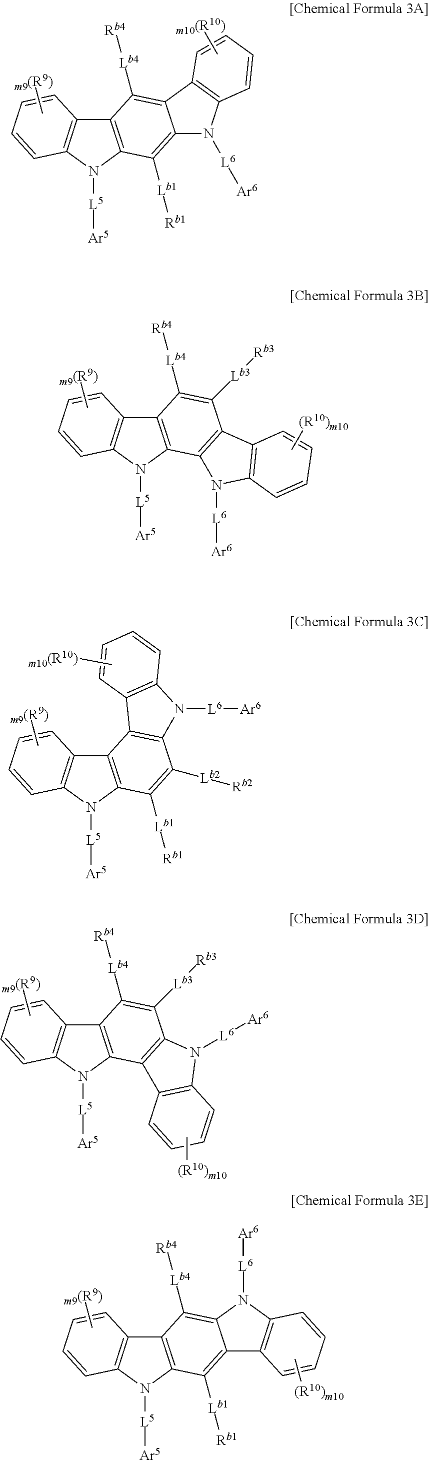

In Chemical Formula 3A to Chemical Formula 3E, Ar⁵, Ar⁶, L⁵, L⁶, R⁹, and R¹⁰ are the same as described above,
L^{b1} to L^{b4} are defined as L⁵ and L⁶ described above, and
R^{b1} to R^{b4} are as defined for R⁹ and R¹⁰ described above.

For example, in Chemical Formulas 3 and 4, Ar⁵ and Ar⁶ may each independently be a substituted or unsubstituted phenyl group, a substituted or unsubstituted biphenyl group, a substituted or unsubstituted naphthyl group, a substituted or unsubstituted triphenylene group, a substituted or unsubstituted carbazolyl group, a substituted or unsubstituted dibenzofuranyl group, or a substituted or unsubstituted dibenzothiophenyl group, and
R^{b1} to R^{b4}, R⁹, and R¹⁰ may each independently be hydrogen, deuterium, a cyano group, a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group.

In a specific embodiment of the present invention, in Chemical Formulas 3 and 4, L⁵-Ar⁵ and L⁶-Ar⁶ may each independently be selected from the substituents listed in Group I.

In an embodiment, R^{b1} to R^{b4}, R⁹, and R¹⁰ may each independently be hydrogen, deuterium, a cyano group, a substituted or unsubstituted phenyl group, a substituted or unsubstituted biphenyl group, a substituted or unsubstituted naphthyl group, a substituted or unsubstituted triphenylene group, a substituted or unsubstituted carbazolyl group, a substituted or unsubstituted dibenzofuranyl group, or a substituted or unsubstituted dibenzothiophenyl group.

For example, R^{b1} to R^{b4}, R⁹, and R¹⁰ may each independently be hydrogen, deuterium, a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group, and

in a specific embodiment, R^{b1} to R^{b4}, R⁹, and R¹⁰ may each independently be hydrogen, deuterium, a phenyl group, a carbazolyl group, a dibenzofuranyl group, or a dibenzothiophenyl group.

For example, in Chemical Formulas 3 and 4, Ar⁵ and Ar⁶ may each independently be a substituted or unsubstituted phenyl group, a substituted or unsubstituted biphenyl group, a substituted or unsubstituted naphthyl group, a substituted or unsubstituted triphenylene group, a substituted or unsubstituted carbazolyl group, a substituted or unsubstituted dibenzofuranyl group, or a substituted or unsubstituted dibenzothiophenyl group, and
at least one of Ar⁵ and Ar⁶ may be a phenyl group substituted or unsubstituted with one or more deuteriums, a biphenyl group substituted or unsubstituted with one or more deuteriums, a naphthyl group substituted or unsubstituted with one or more deuteriums, a triphenylene group substituted or unsubstituted with one or more deuteriums, a carbazolyl group substituted or unsubstituted with one or more deuteriums, a dibenzofuranyl group substituted or unsubstituted with one or more deuteriums, or a dibenzothiophenyl group substituted or unsubstituted with one or more deuteriums.

For example, in Chemical Formulas 3 and 4, R^{b1} to R^{b4}, R⁹, and R¹⁰ may each independently be hydrogen, deuterium, a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group, and
at least one of R^{b1} to R^{b4}, R⁹, and R¹⁰ may be deuterium, a phenyl group substituted or unsubstituted with one or more deuteriums, a biphenyl group substituted or unsubstituted with one or more deuteriums, a carbazolyl group substituted or unsubstituted with one or more deuteriums, a dibenzofuranyl group substituted or unsubstituted with one or more deuteriums, or a dibenzothiophenyl group substituted or unsubstituted with one or more deuteriums.

For example, in Chemical Formulas 3 and 4, at least one of Ar⁵ and Ar⁶ may be a phenyl group substituted or unsubstituted with one or more deuteriums, a biphenyl group substituted or unsubstituted with one or more deuteriums, a naphthyl group substituted or unsubstituted with one or more deuteriums, a triphenylene group substituted or unsubstituted with one or more deuteriums, a carbazolyl group substituted or unsubstituted with one or more deuteriums, a dibenzofuranyl group substituted or unsubstituted with one or more deuteriums, or a dibenzothiophenyl group substituted or unsubstituted with one or more deuteriums, and
at least one of R^{b1} to R^{b4}, R⁹ and R¹⁰ may be deuterium, a phenyl group substituted or unsubstituted with one or more deuteriums, a biphenyl group substituted or unsubstituted with one or more deuteriums, a carbazolyl group substituted or unsubstituted with one or more deuteriums, a dibenzofuranyl group substituted or unsubstituted with one or more deuteriums, or a dibenzothiophenyl group substituted or unsubstituted with one or more deuteriums.

In a specific embodiment of the present invention, the second compound may be represented by Chemical Formula 2-8, wherein in Chemical Formula 2-8, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted carbazolyl group, a substituted or unsubstituted dibenzothiophenyl group, a substituted or unsubstituted dibenzofuranyl group, or a substituted or unsubstituted fluorenyl group, and L³ and L⁴ may each independently be a single bond, or a substituted or unsubstituted C6 to C20 arylene group, R⁴ to R⁸ are each independently hydrogen, deuterium, a cyano group, a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group.

In a specific embodiment of the present invention, the second compound may be represented by Chemical Formula 3C, wherein, in Chemical Formula Chemical Formula 3C, L^{b1} and L^{b2} may be a single bond, L⁵ and L⁶ may each independently be a single bond or a substituted or unsubstituted C6 to C12 arylene group, R^{b1}, R^{b2}, R⁹, and R¹⁰ may each independently be hydrogen, deuterium, a phenyl group, a triphenylene group, a carbazolyl group, a dibenzofuranyl group, or a dibenzothiophenyl group, and Ar⁵ and Ar⁶ may each independently be a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group.

For example, the second compound may be one selected from the compounds listed in Group 2, but is not limited thereto.

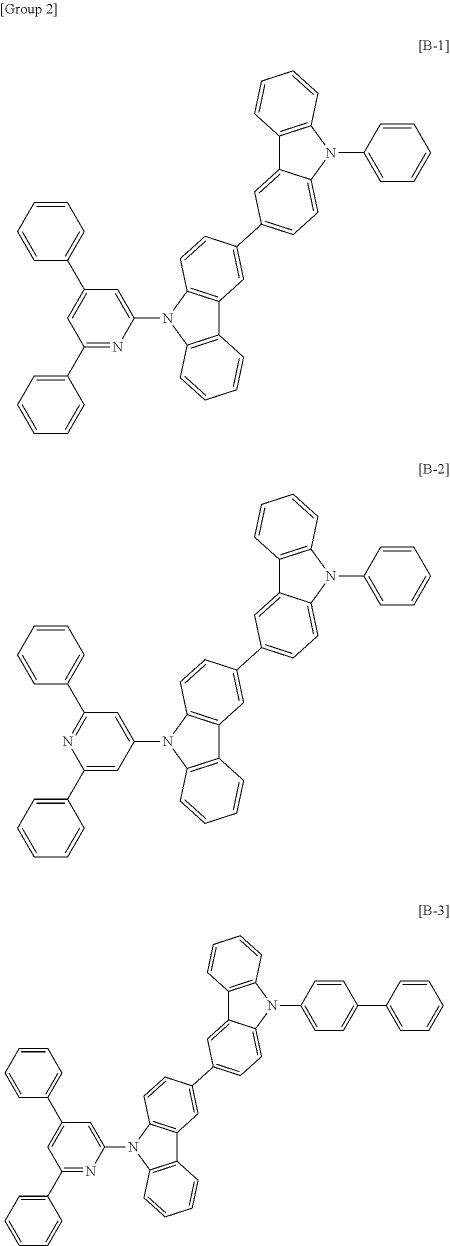 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

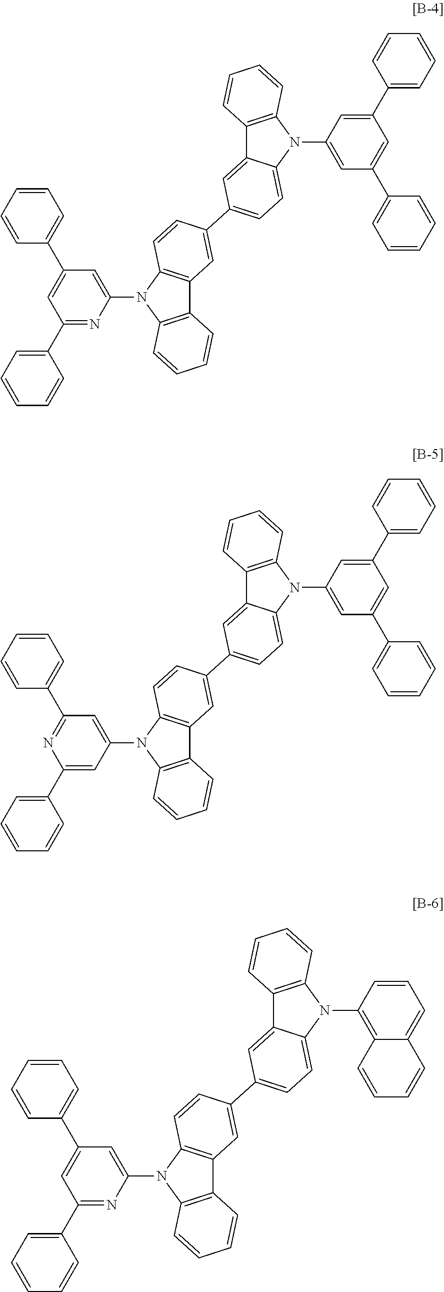 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

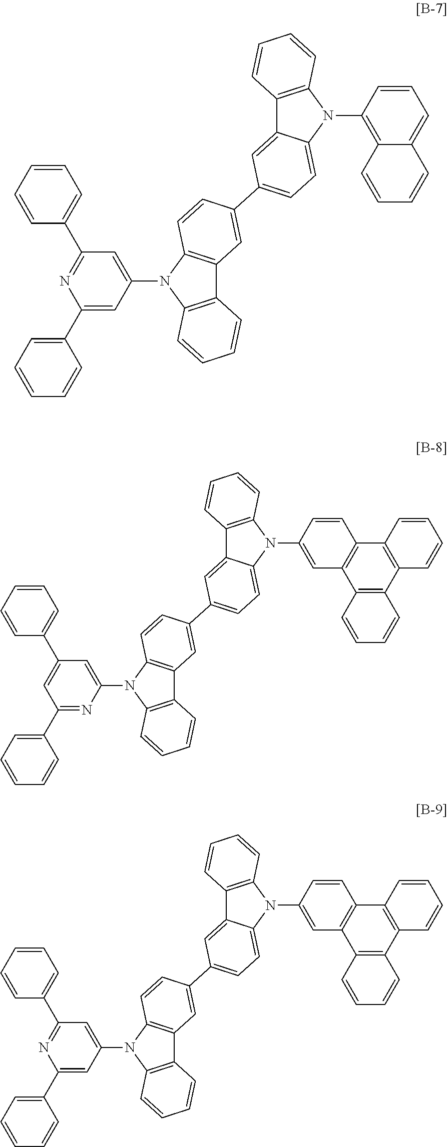 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1]

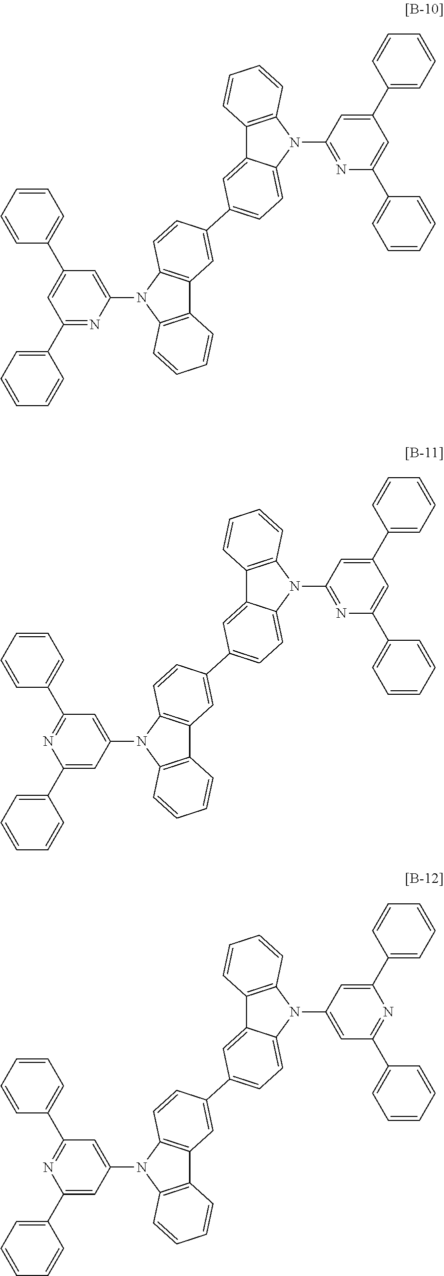 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

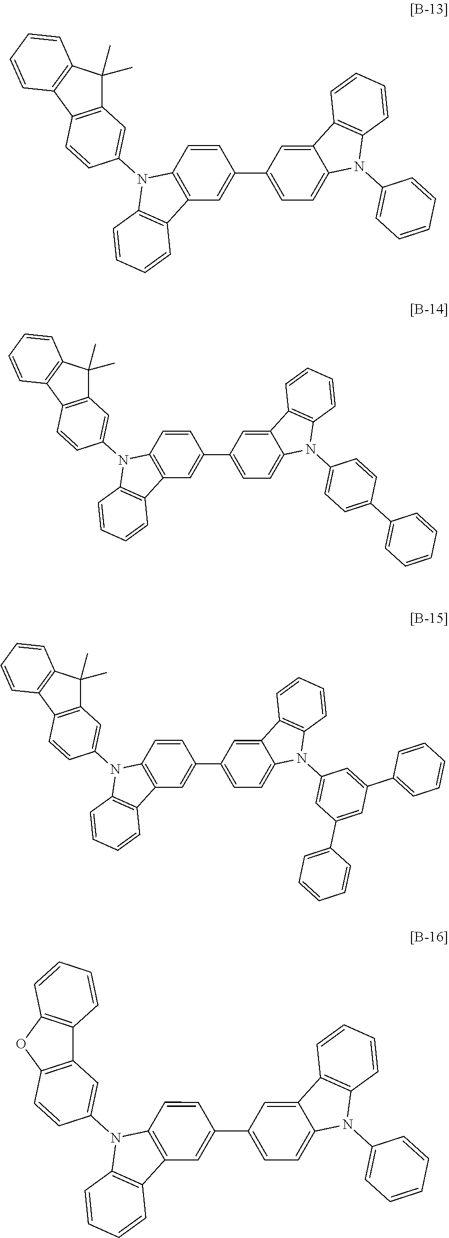 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1]

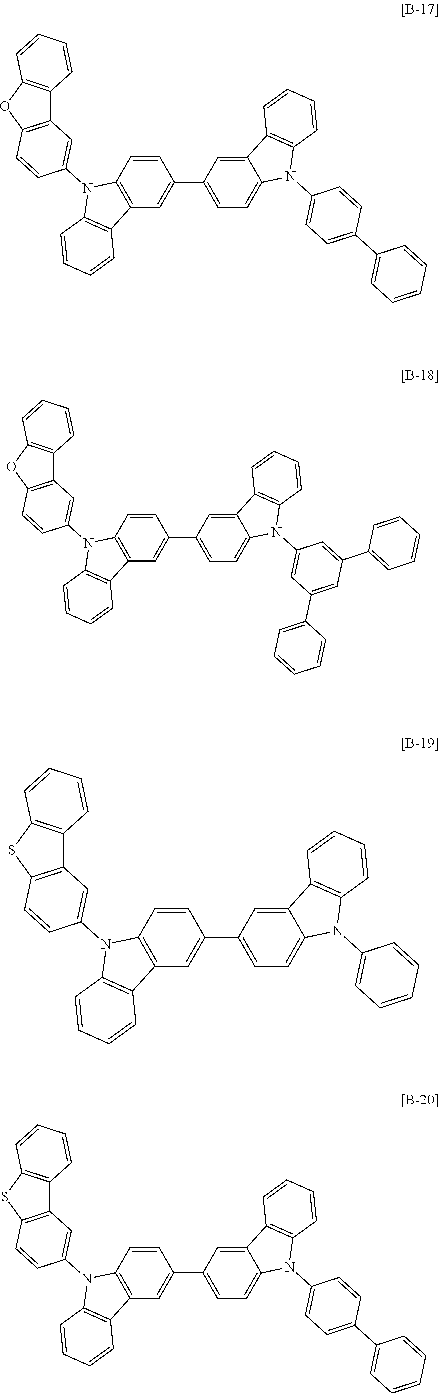 [SMILES: c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

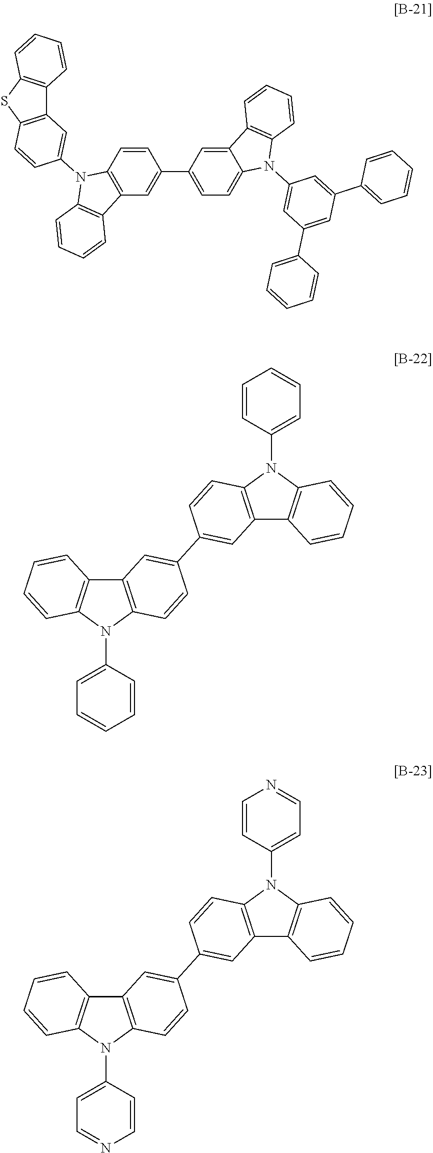 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1]

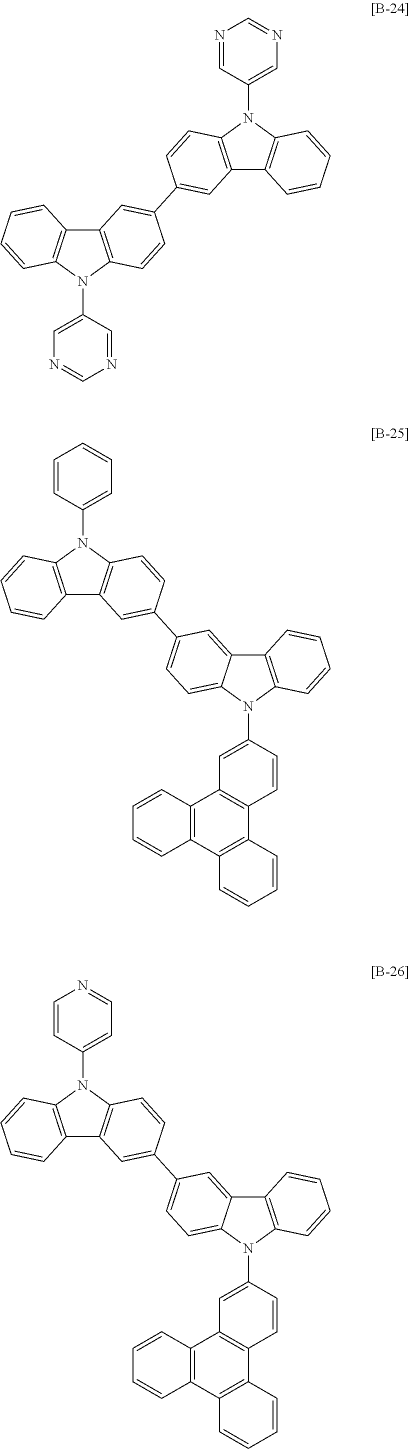 [SMILES: c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

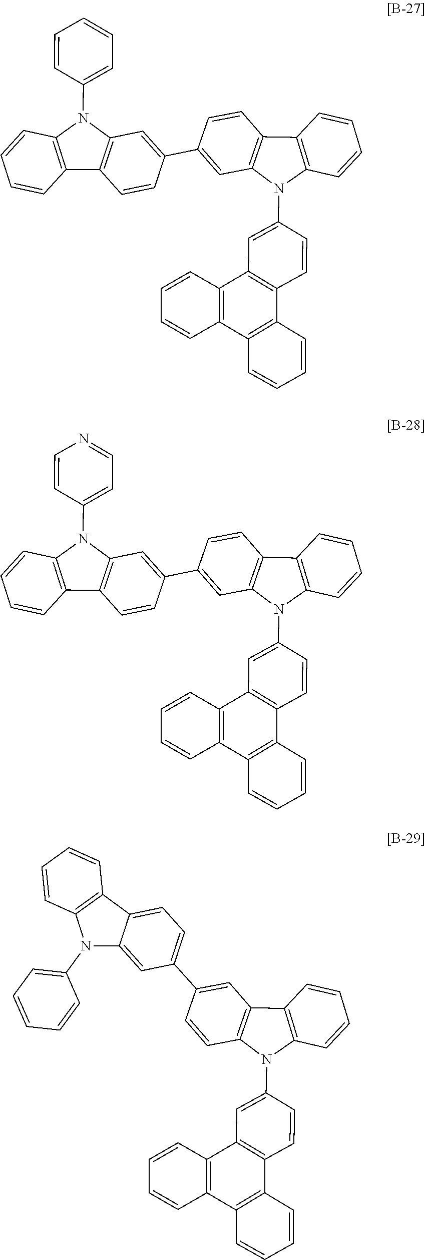 [SMILES: c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1]

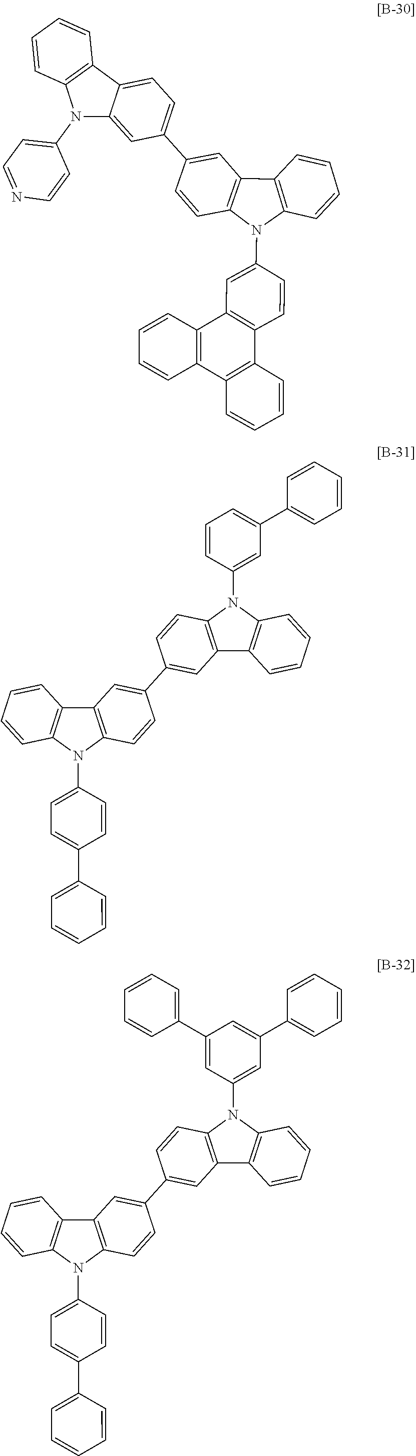 [SMILES: c1ccc2c(c1)c1ccccc1c1cc(-n3c4ccccc4c4cc(-c5ccc6c7ccccc7n(-c7ccncc7)c6c5)ccc43)ccc21 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

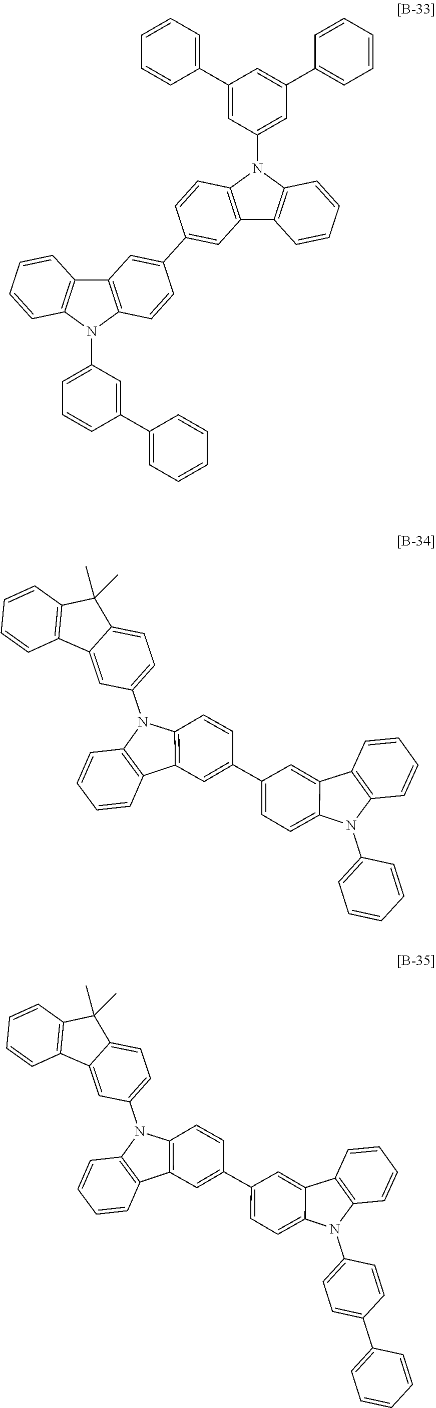 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

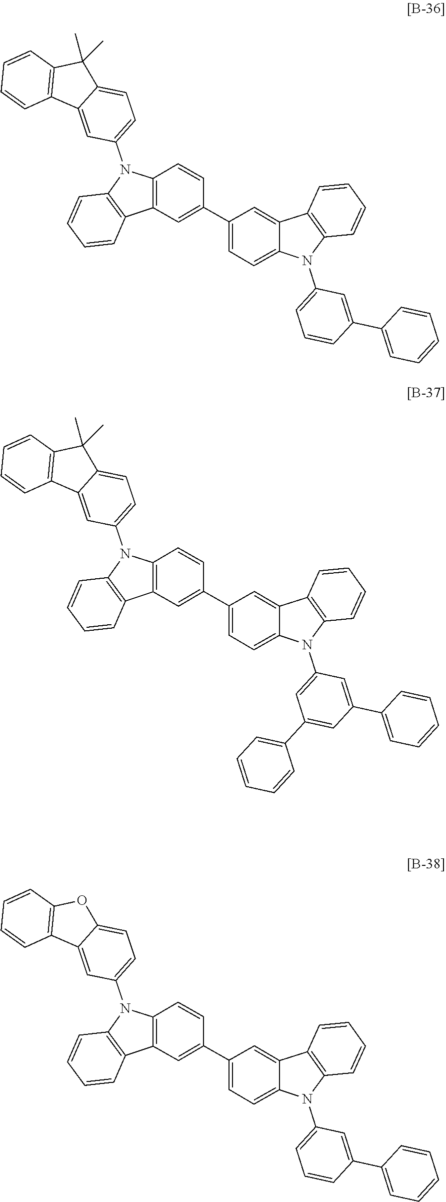 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1]

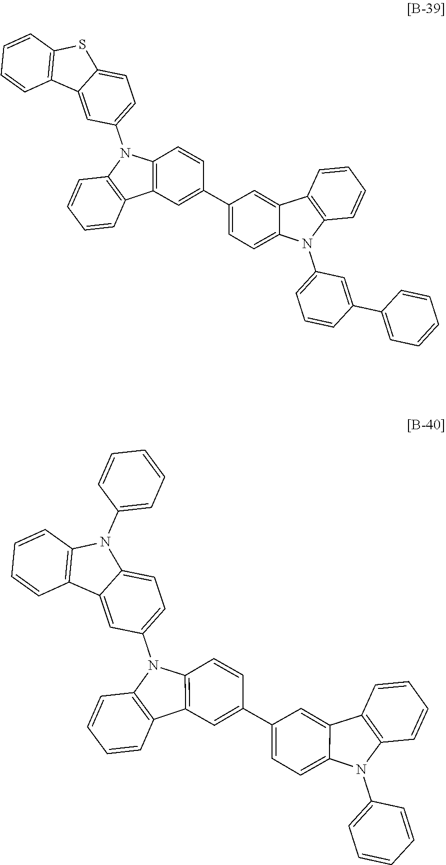 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

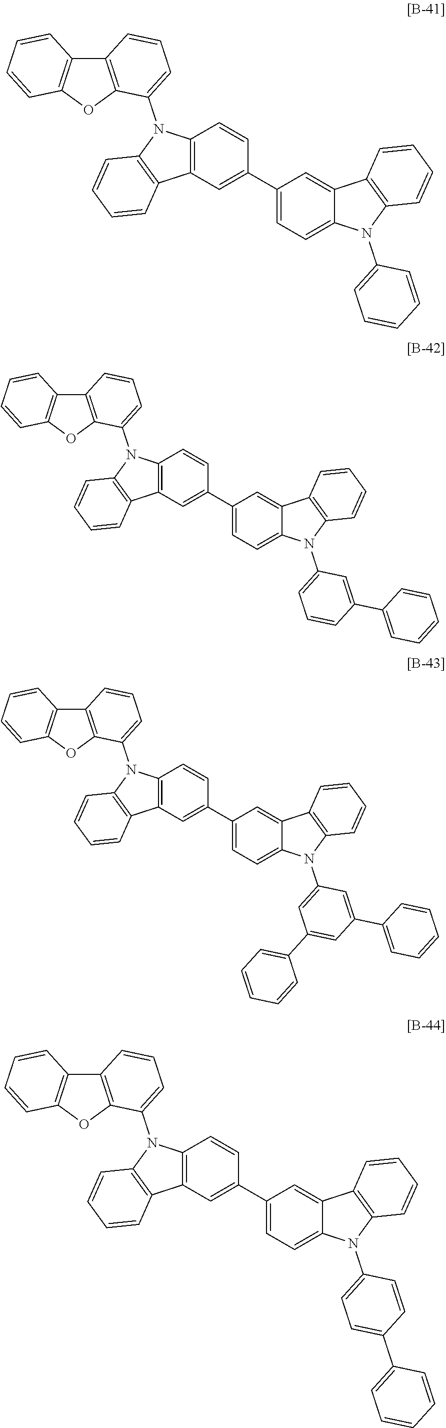 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1]

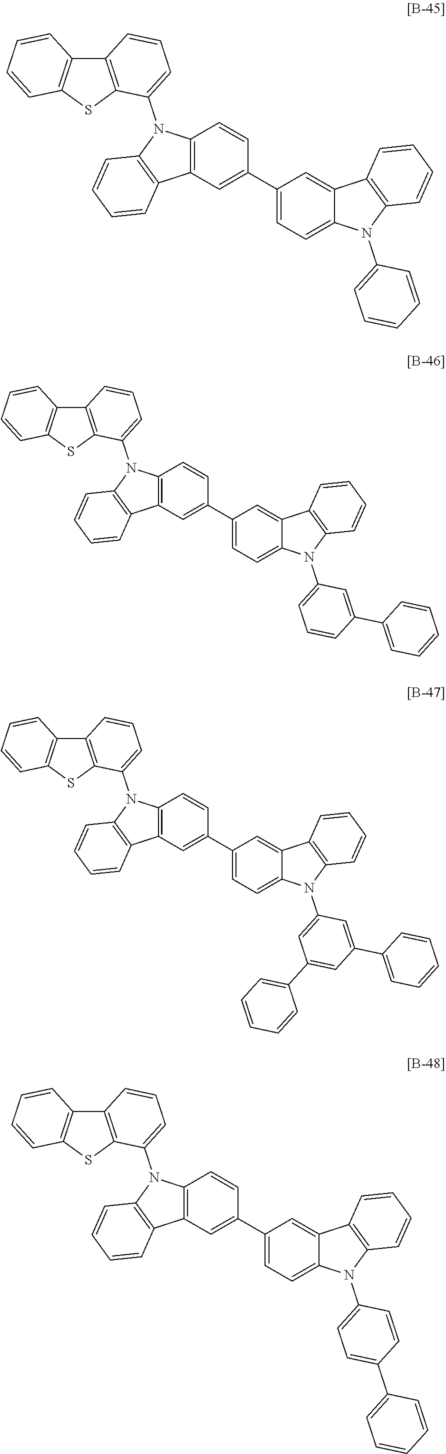 [SMILES: c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

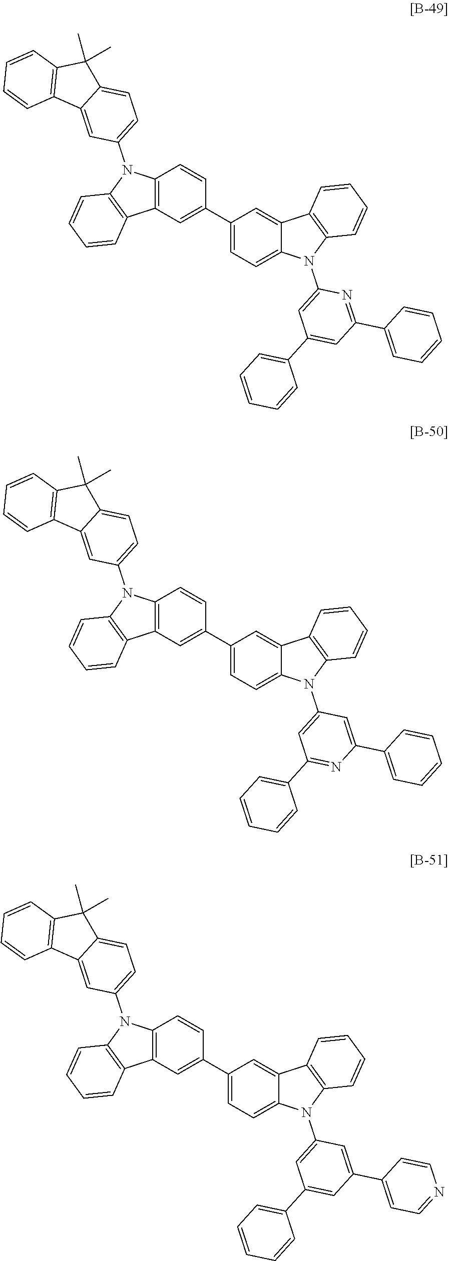 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21]

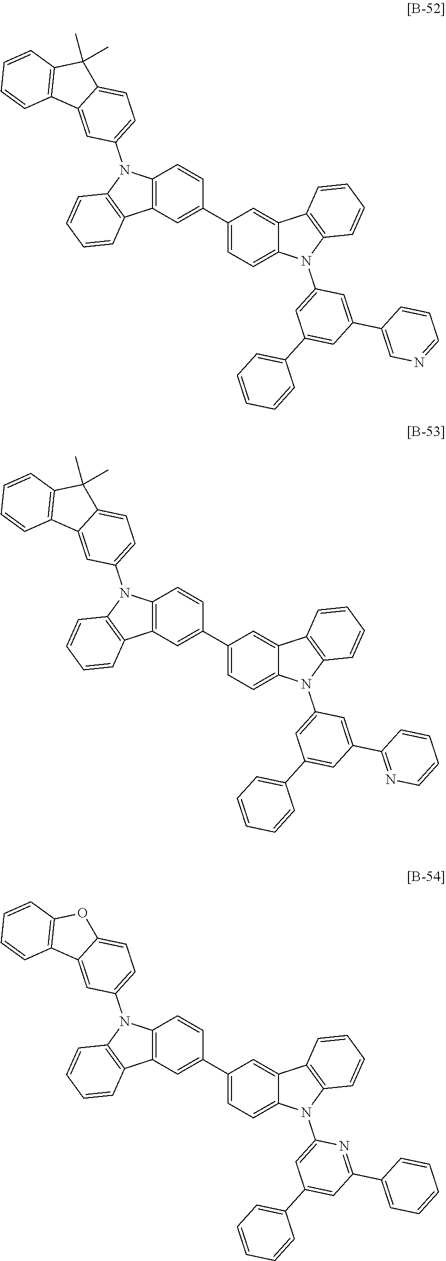 [SMILES: CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21 ; c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1]

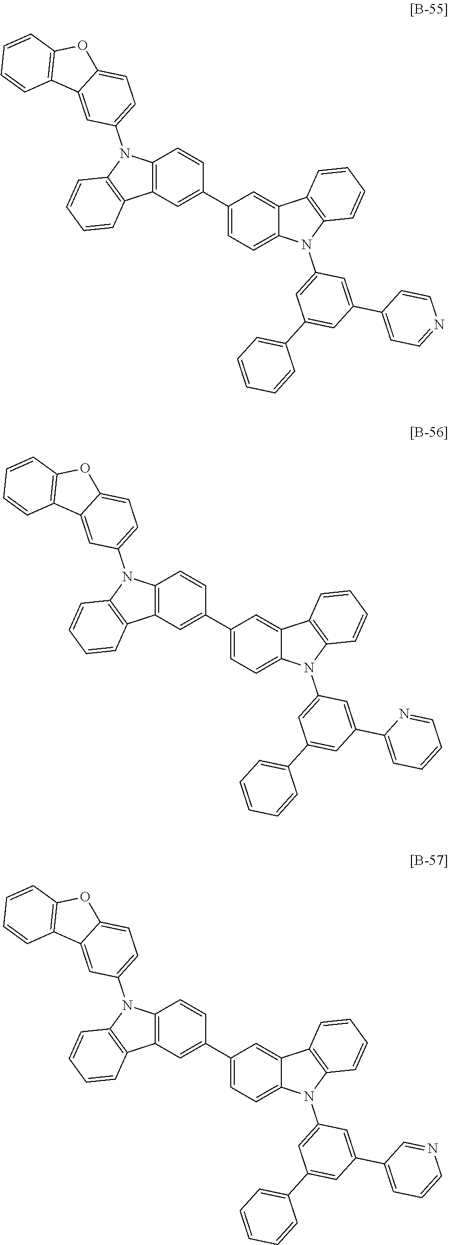 [SMILES: c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1]

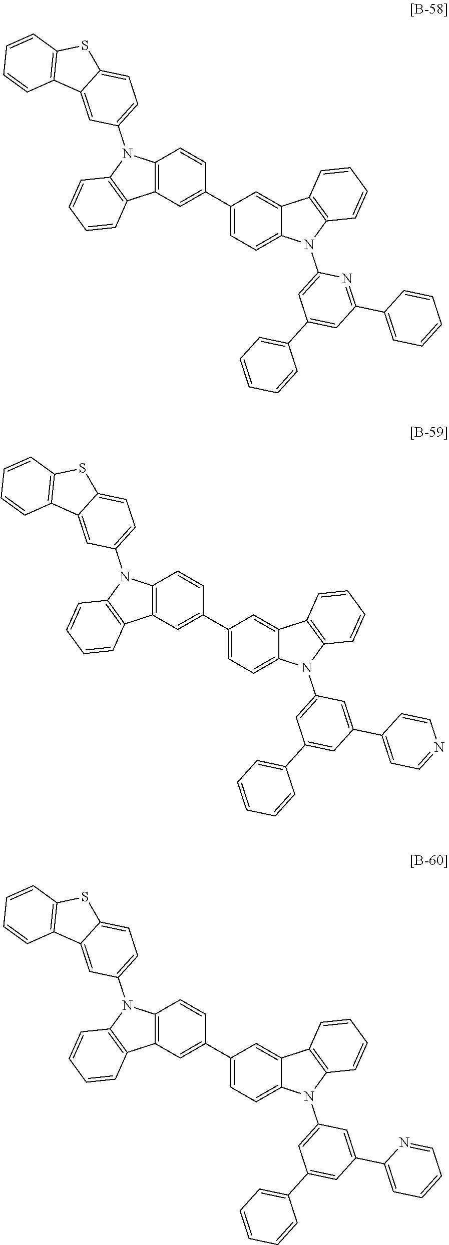 [SMILES: c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

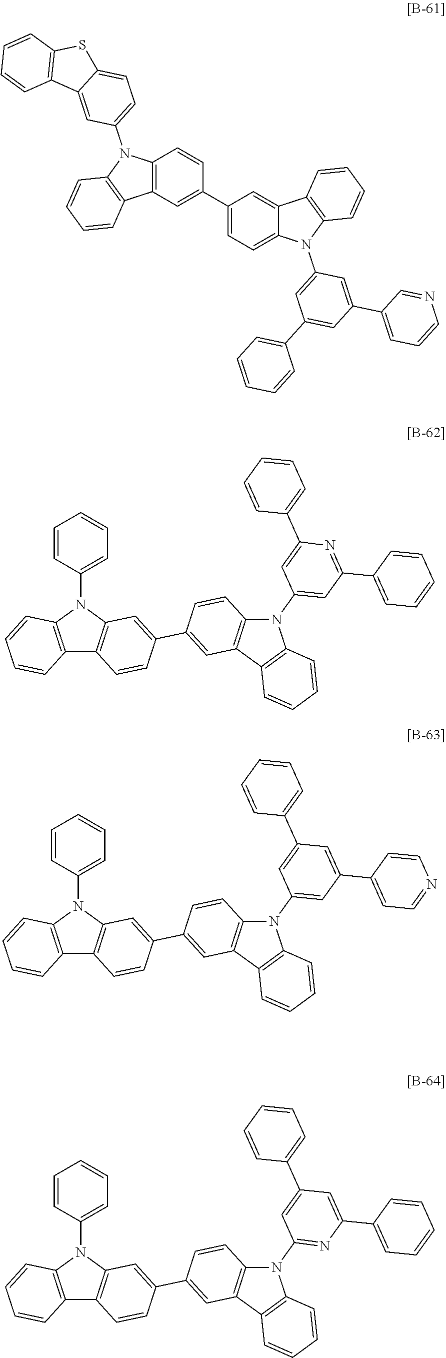 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1]

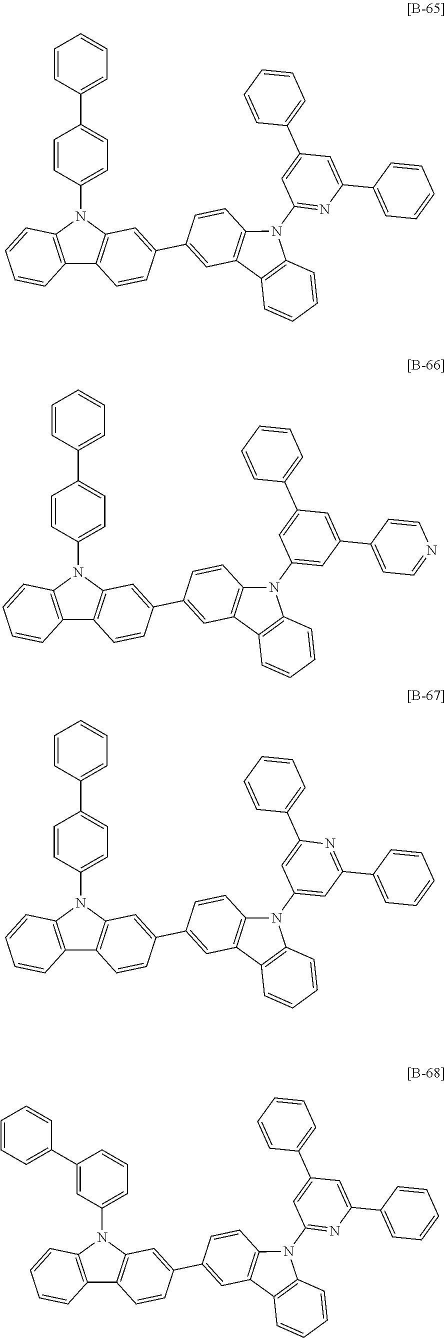 [SMILES: c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

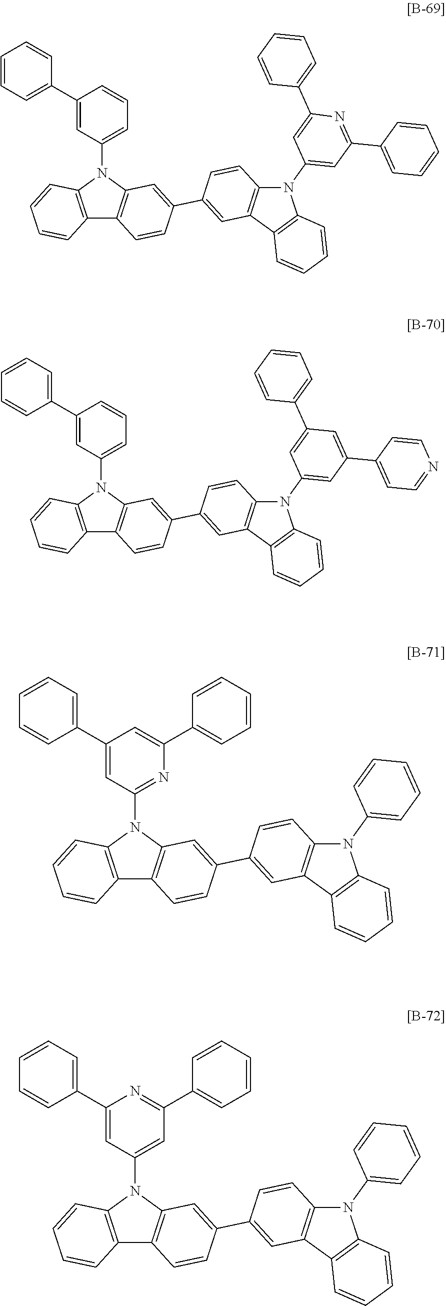 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1]

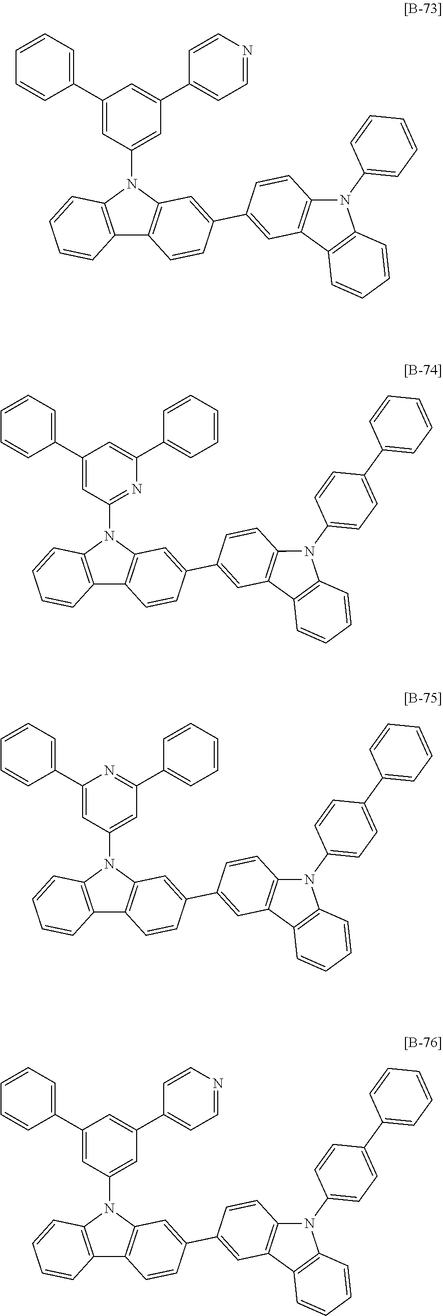 [SMILES: c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1]

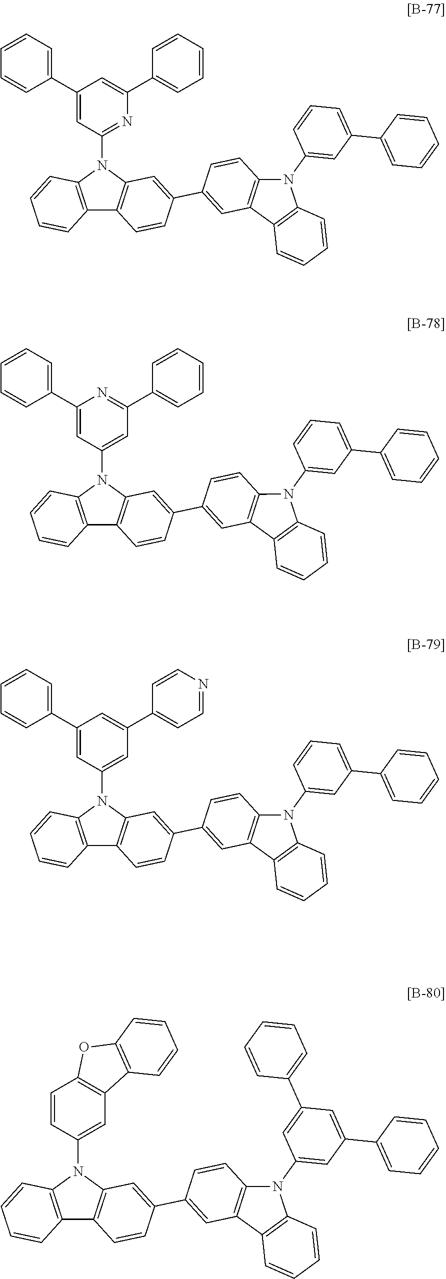 [SMILES: c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

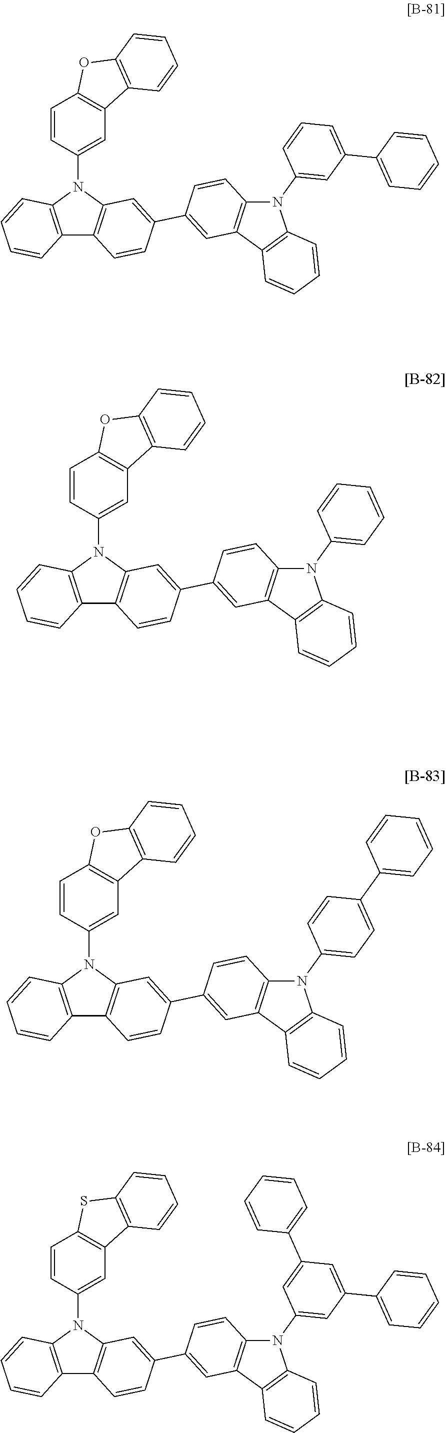 [SMILES: c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1]

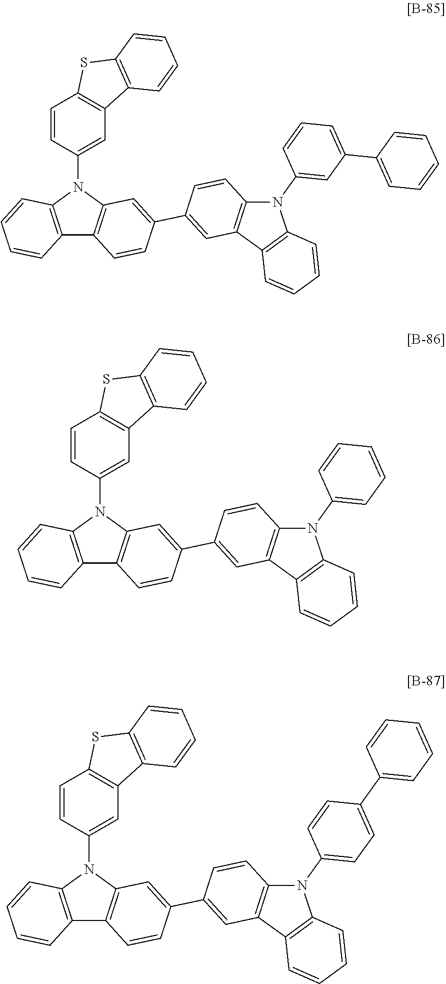 [SMILES: c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1]

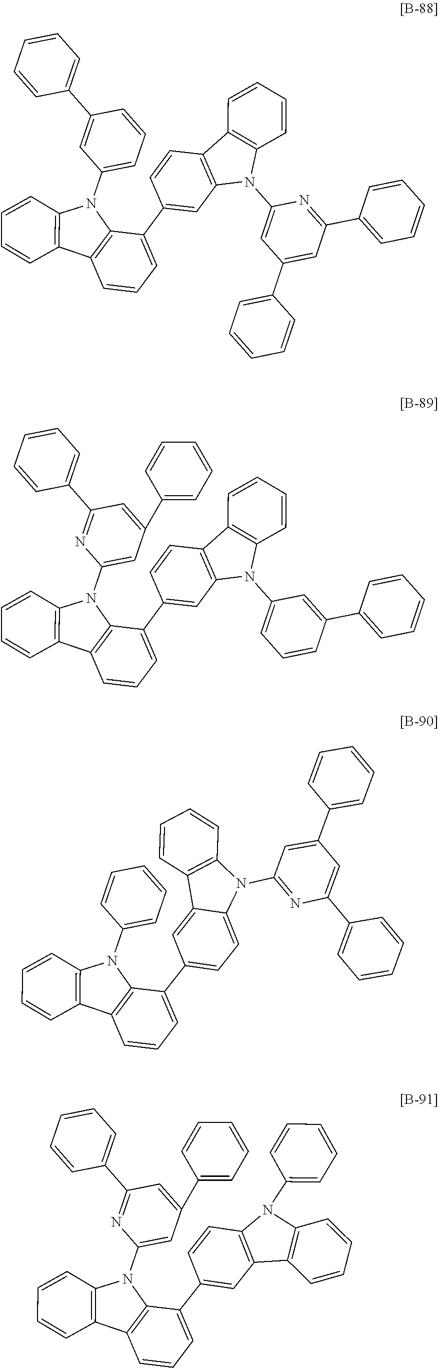 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1 ; c1ccc(-c2cc(-c3ccccc3)nc(-n3c4ccccc4c4cccc(-c5ccc6c(c5)c5ccccc5n6-c5ccccc5)c43)c2)cc1]

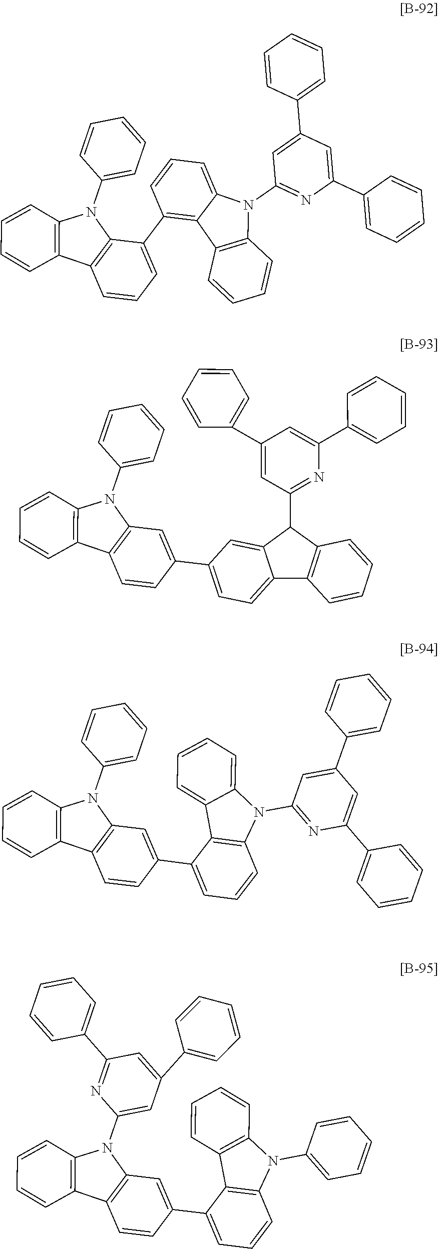 [SMILES: c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(C3c4ccccc4-c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1]

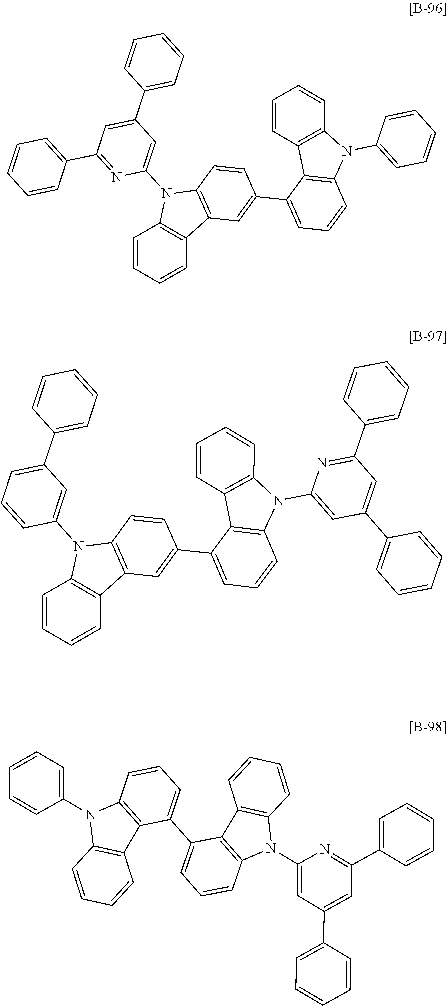 [SMILES: c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

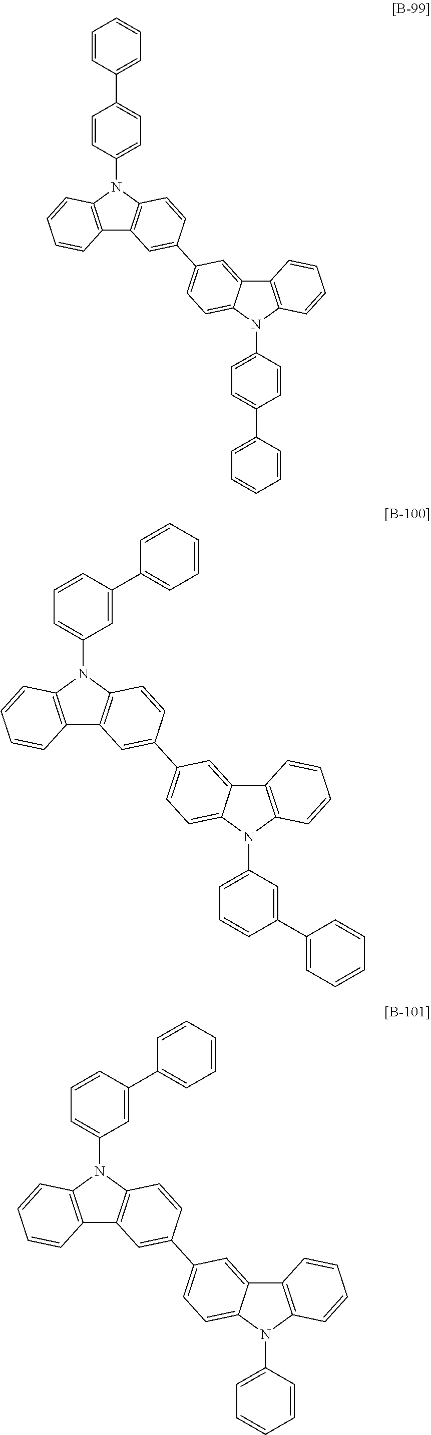 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1]

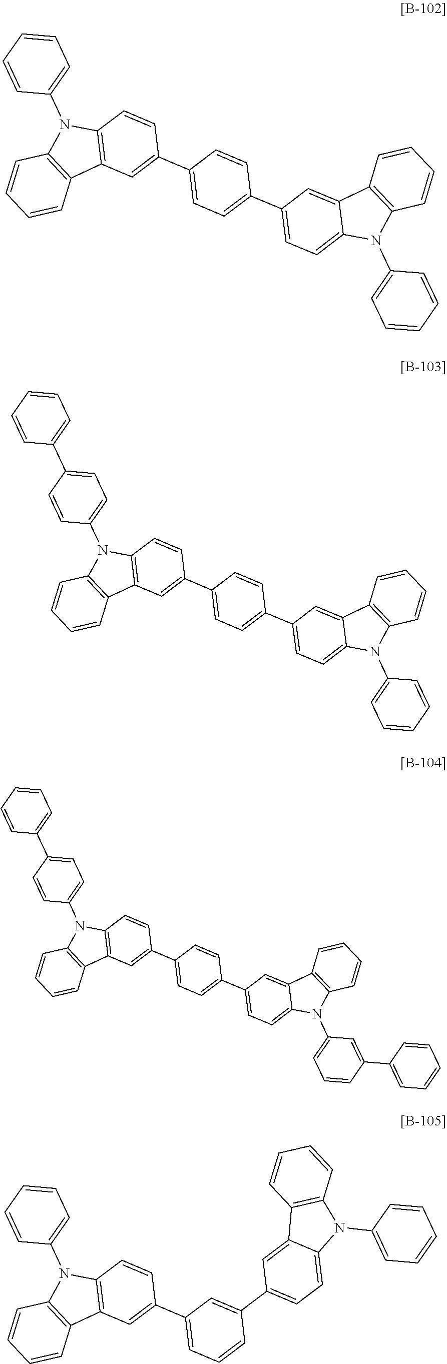 [SMILES: c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1]

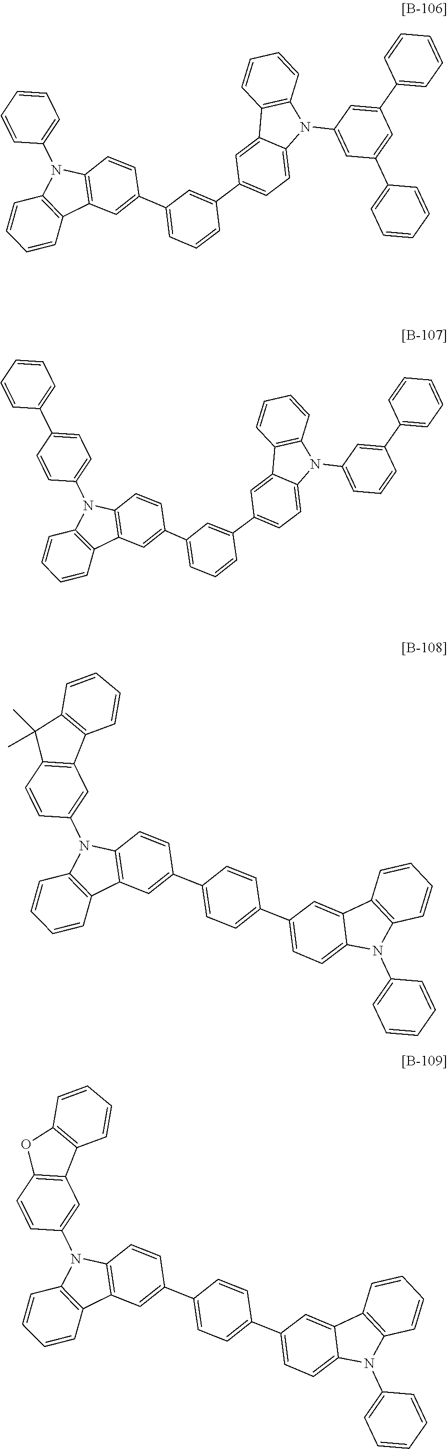 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21 ; c1ccc(-n2c3ccccc3c3cc(-c4ccc(-c5ccc6c(c5)c5ccccc5n6-c5ccc6oc7ccccc7c6c5)cc4)ccc32)cc1]

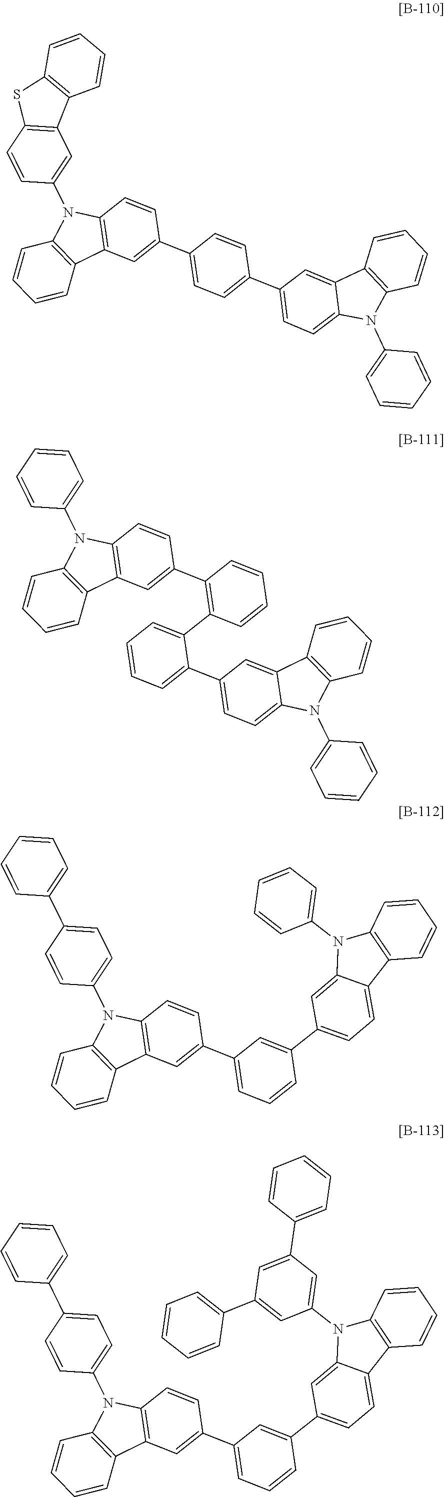 [SMILES: c1ccc(-n2c3ccccc3c3cc(-c4ccc(-c5ccc6c(c5)c5ccccc5n6-c5ccc6sc7ccccc7c6c5)cc4)ccc32)cc1 ; c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1]

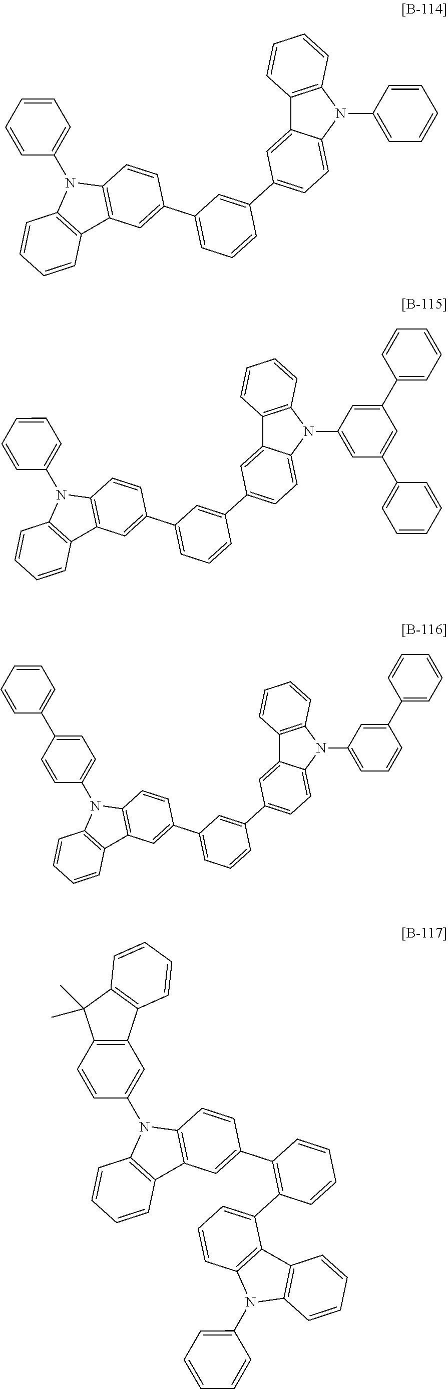 [SMILES: c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21]

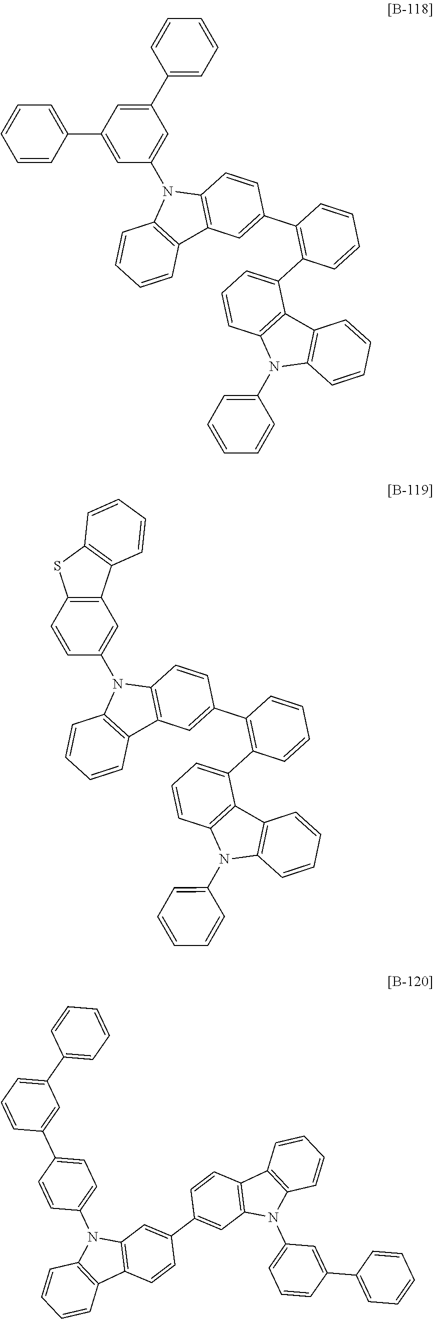 [SMILES: c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

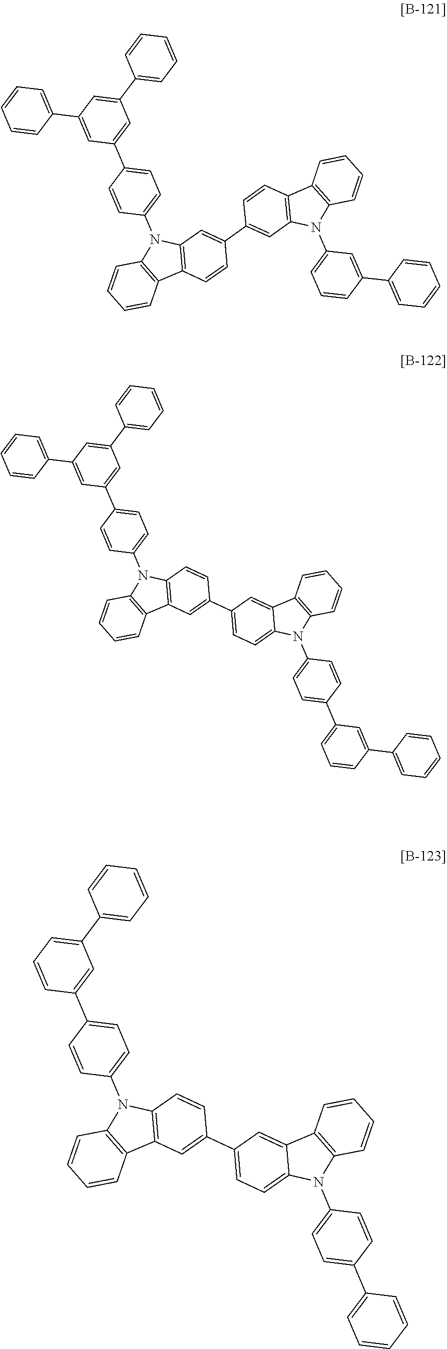 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

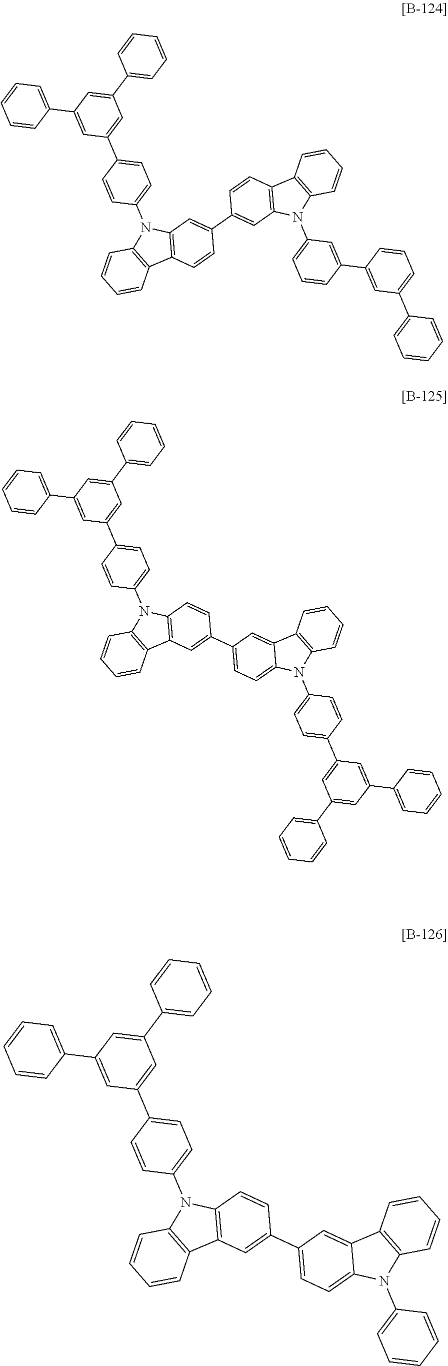 [SMILES: c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

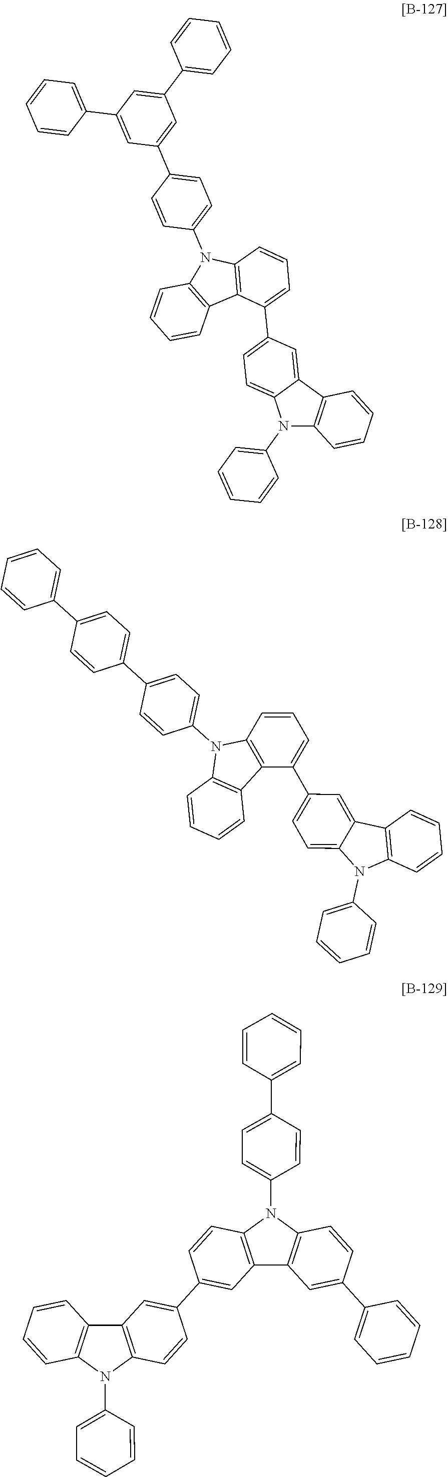 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

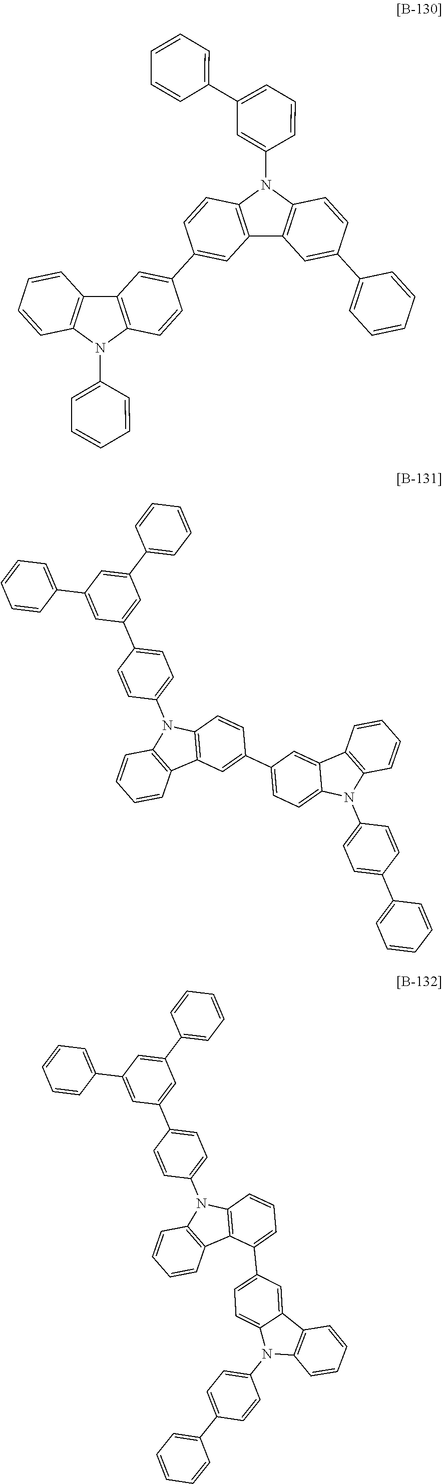 [SMILES: c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1]

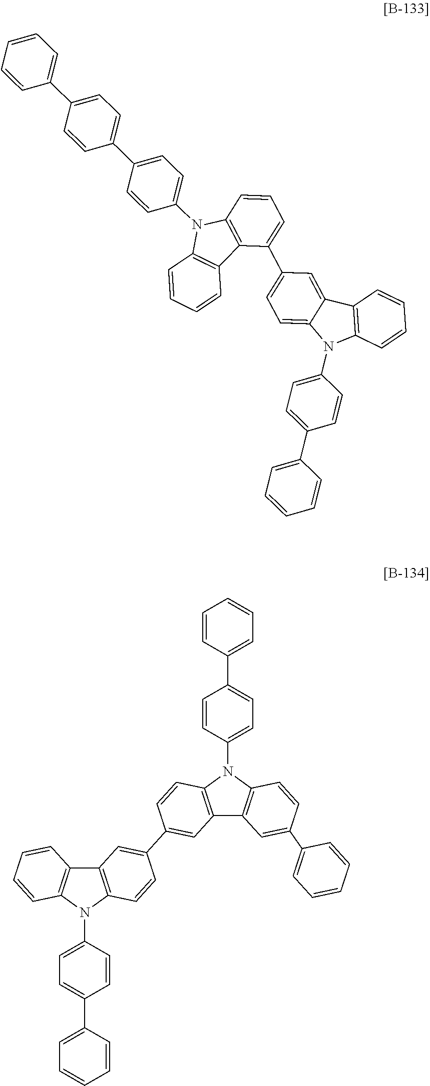 [SMILES: c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

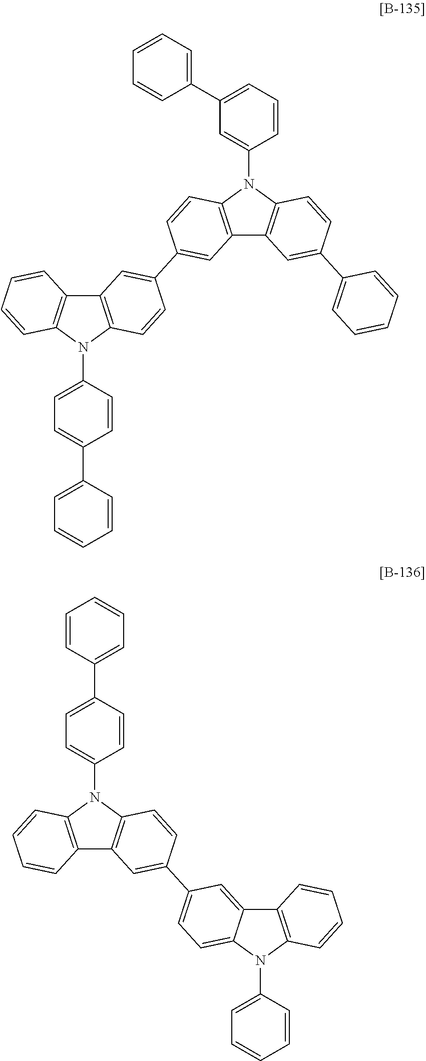 [SMILES: c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1]

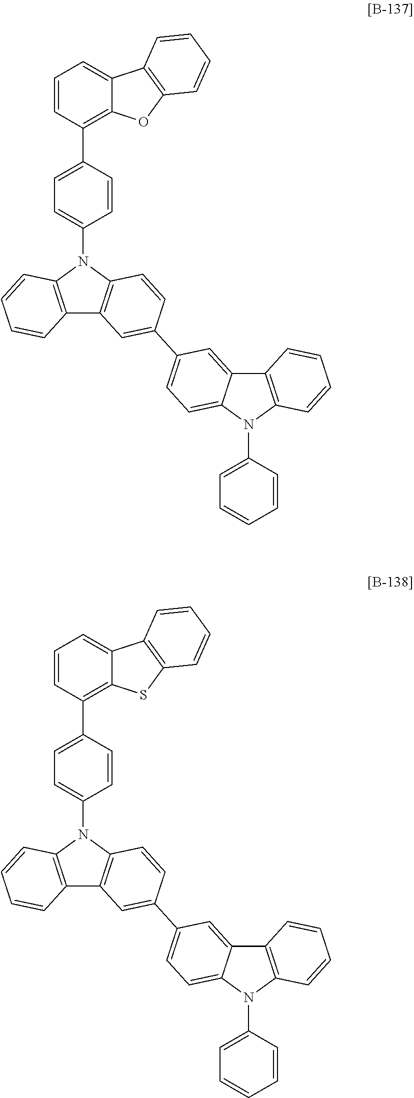 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

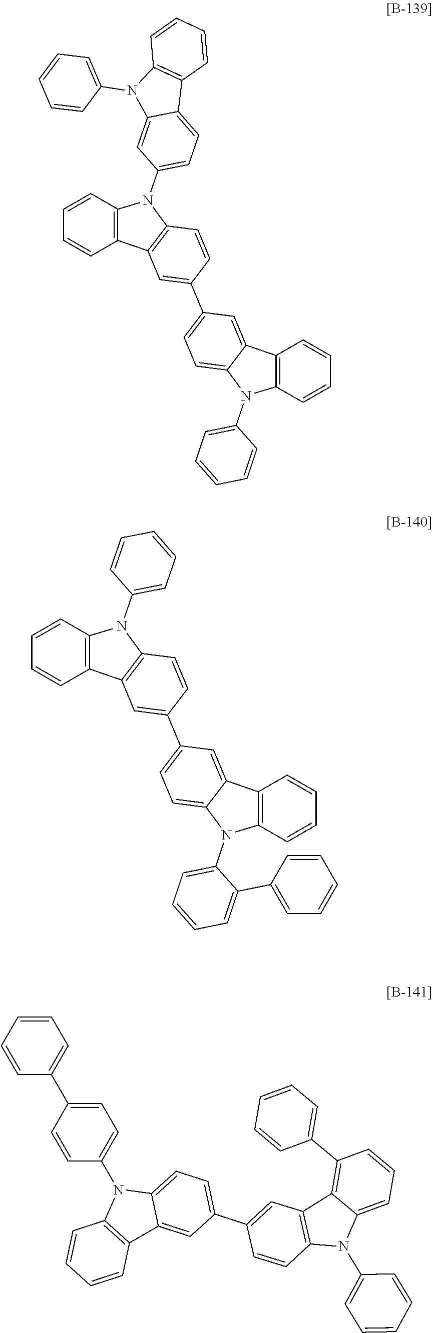 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1]

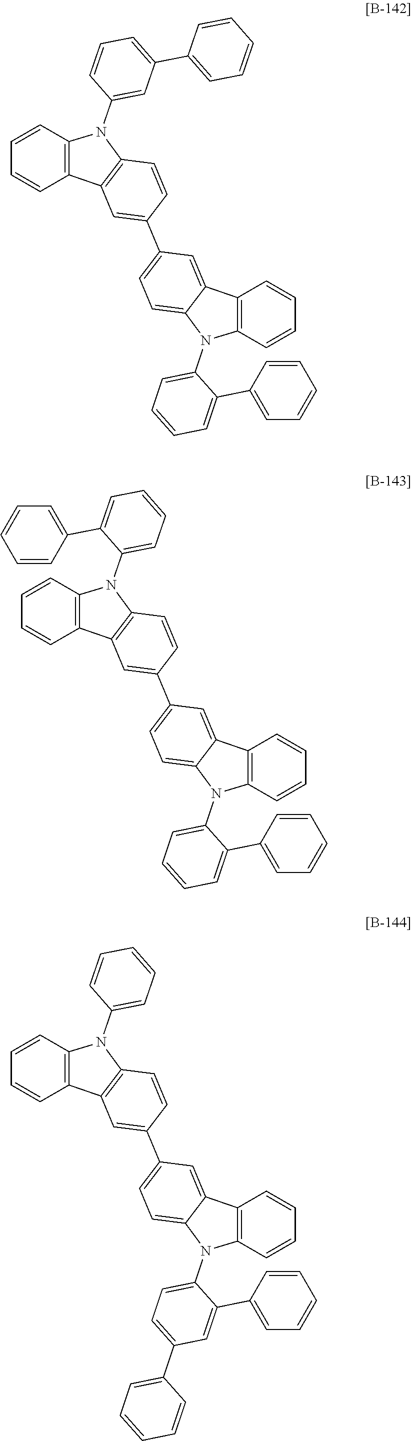 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1]

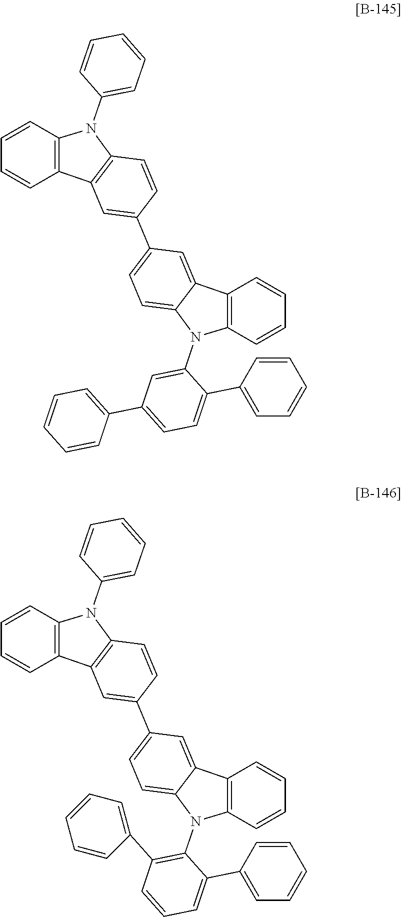 [SMILES: c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1]

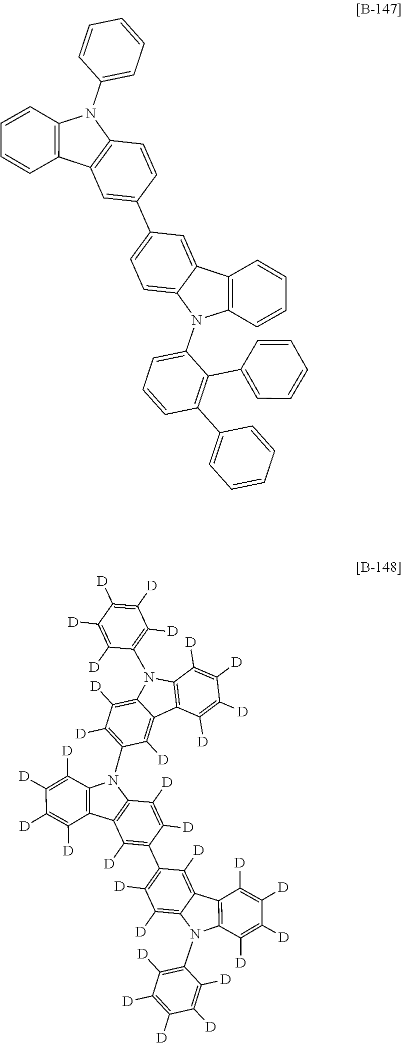 [SMILES: c1ccc(-c2cccc(-n3c4ccccc4c4cc(-c5ccc6c(c5)c5ccccc5n6-c5ccccc5)ccc43)c2-c2ccccc2)cc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

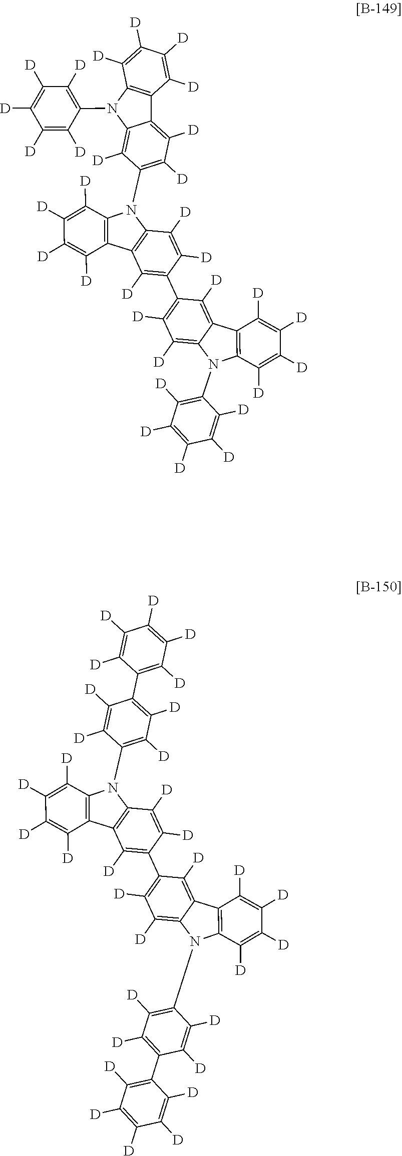

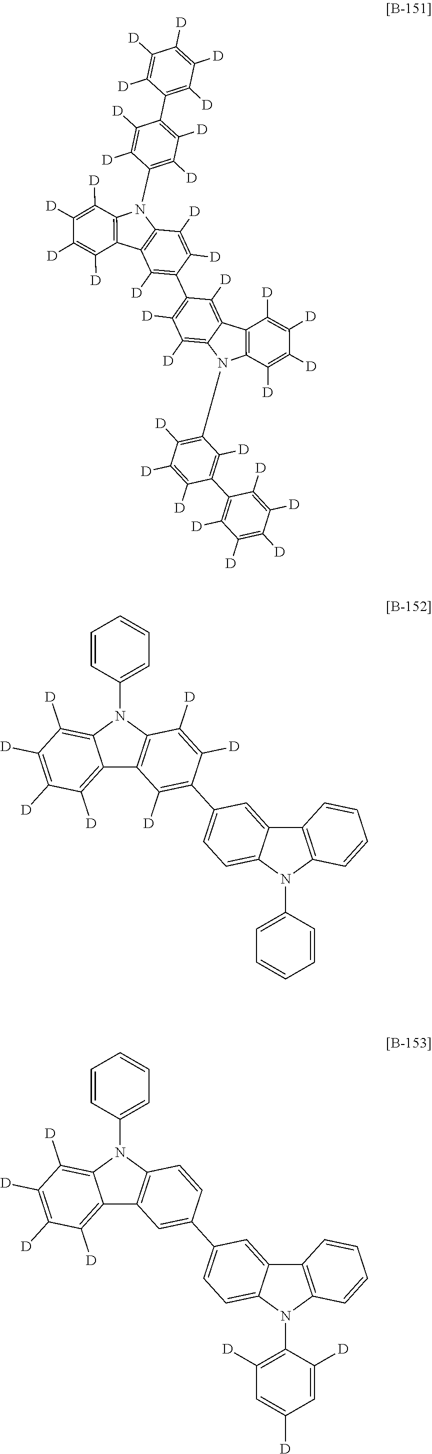 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H] ; [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1]

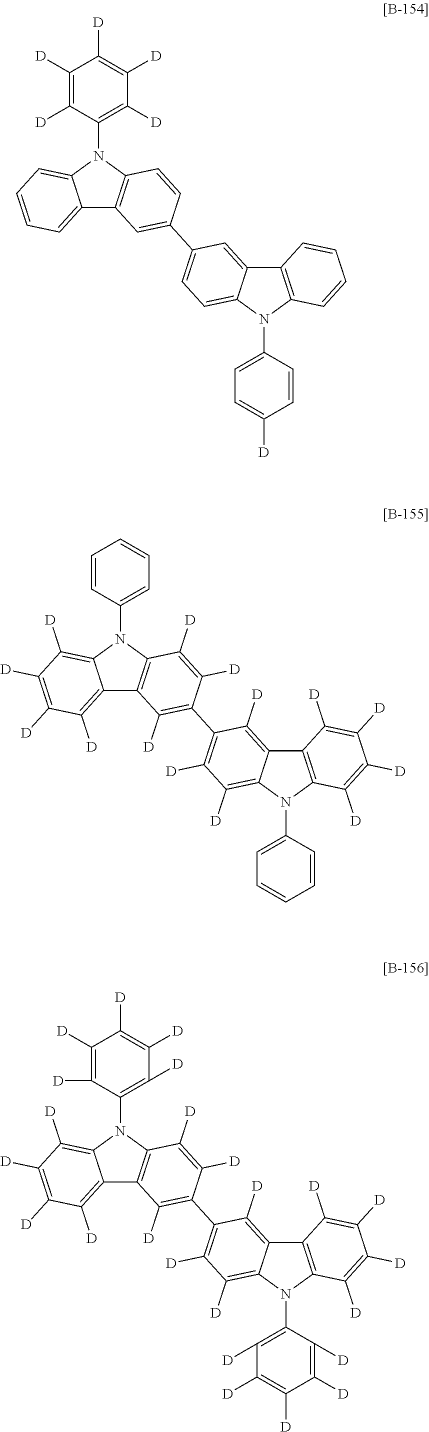 [SMILES: [2H]c1ccc(-n2c3ccccc3c3cc(-c4ccc5c(c4)c4ccccc4n5-c4c([2H])c([2H])c([2H])c([2H])c4[2H])ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

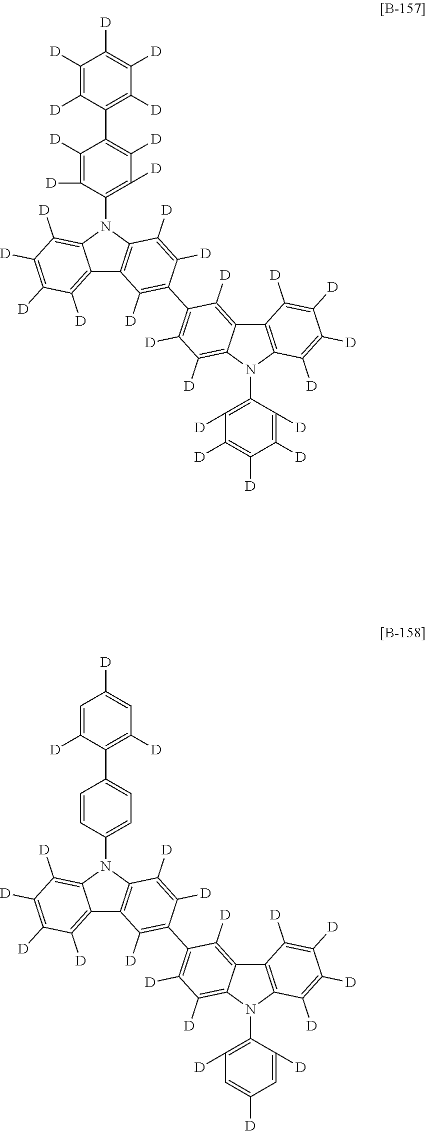 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H] ; [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1]

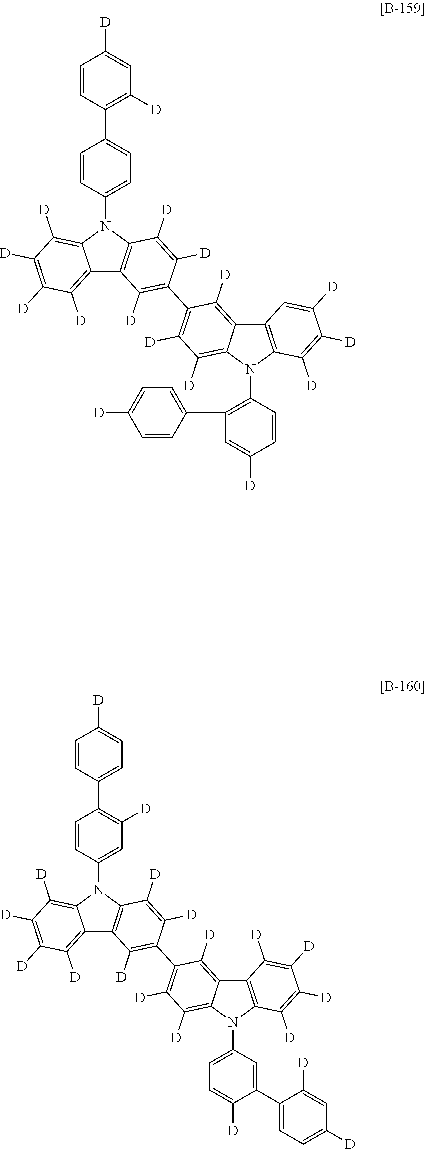 [SMILES: [2H]c1ccc(-c2cc([2H])ccc2-n2c3c([2H])c([2H])c([2H])cc3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1 ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1]

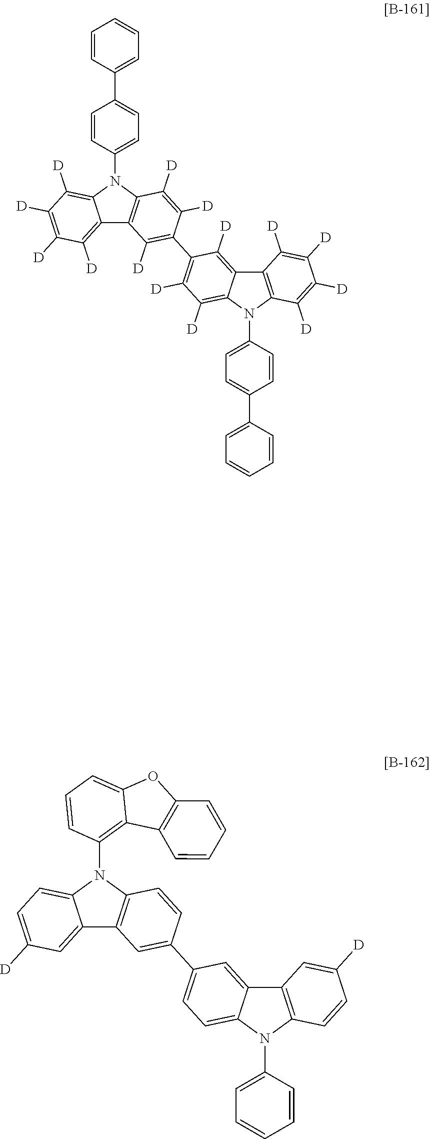 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; [2H]c1ccc2c(c1)c1cc(-c3ccc4c(c3)c3cc([2H])ccc3n4-c3cccc4oc5ccccc5c34)ccc1n2-c1ccccc1]

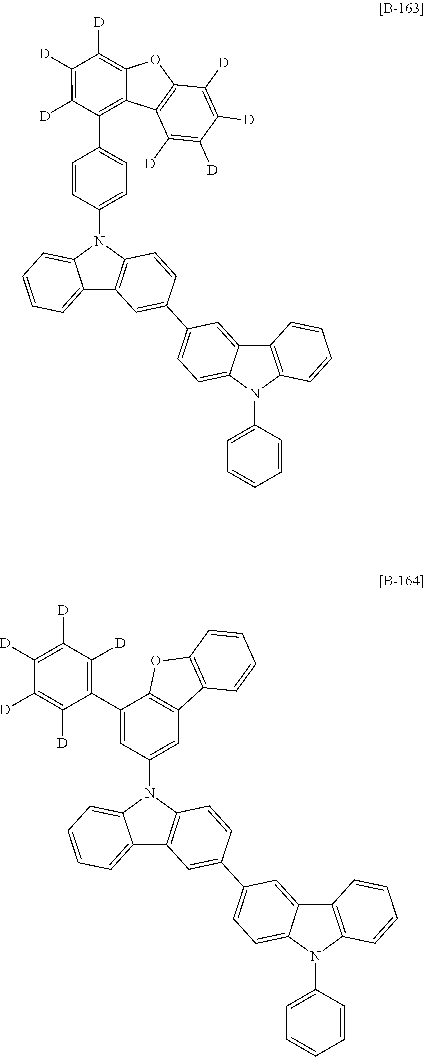 [SMILES: [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

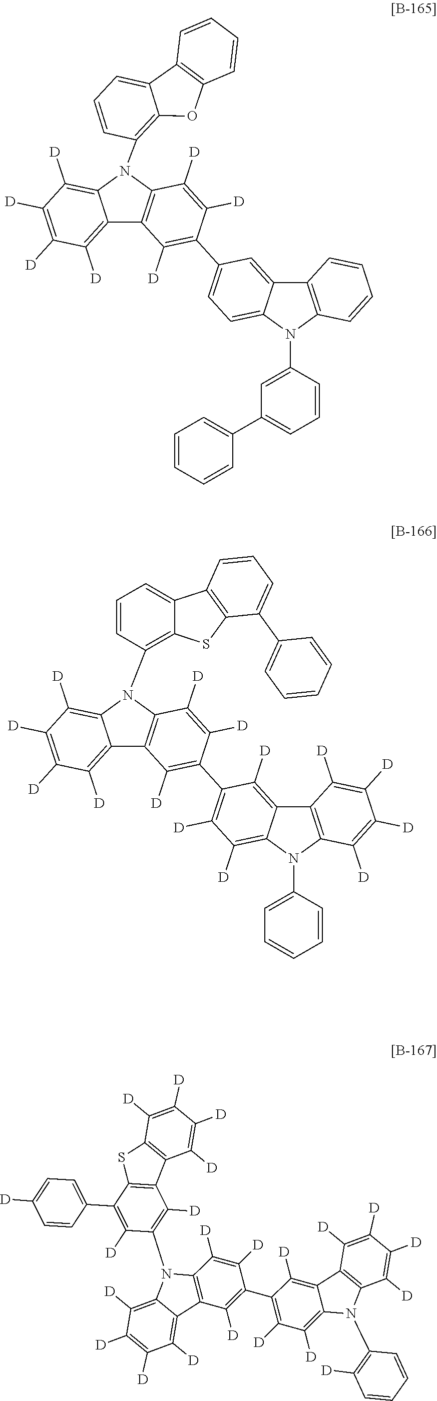

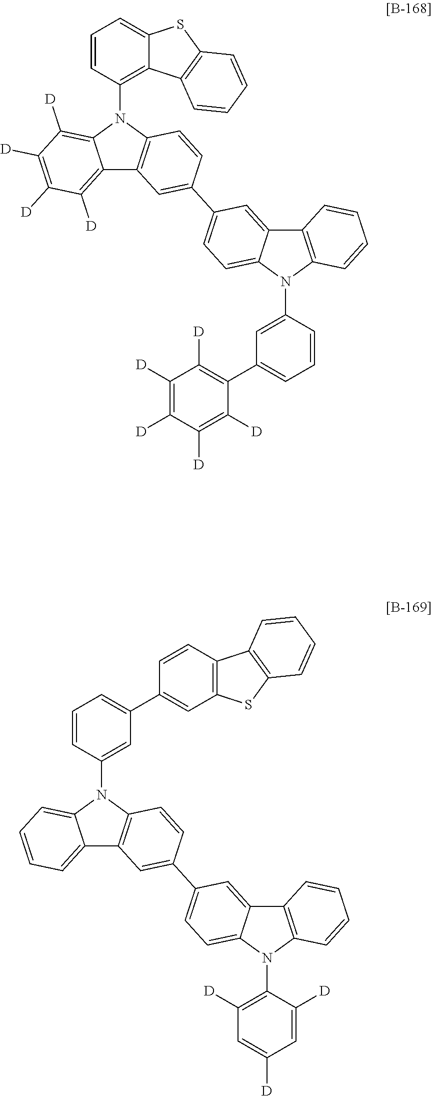 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

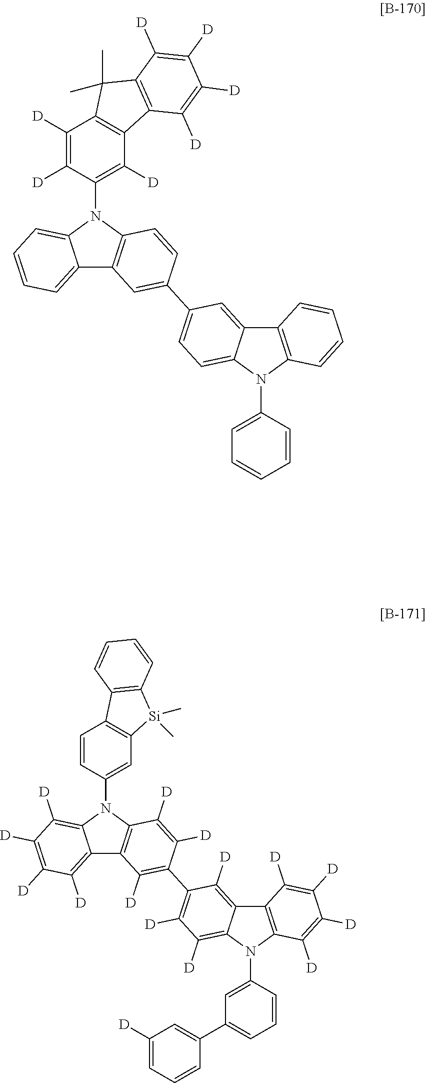 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

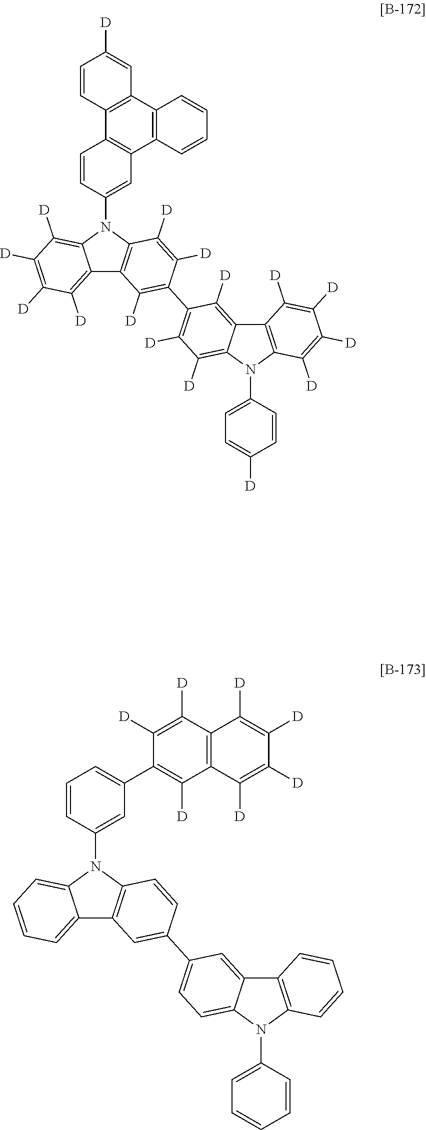 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H]]

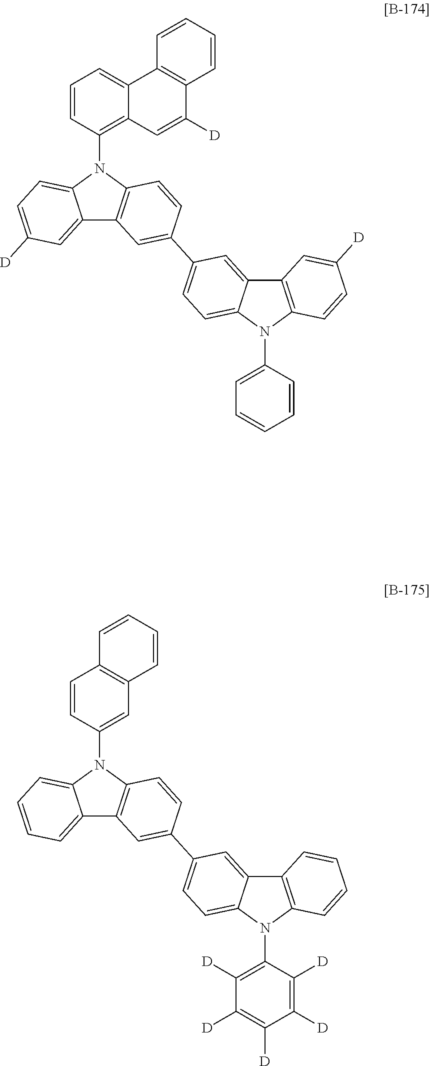 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

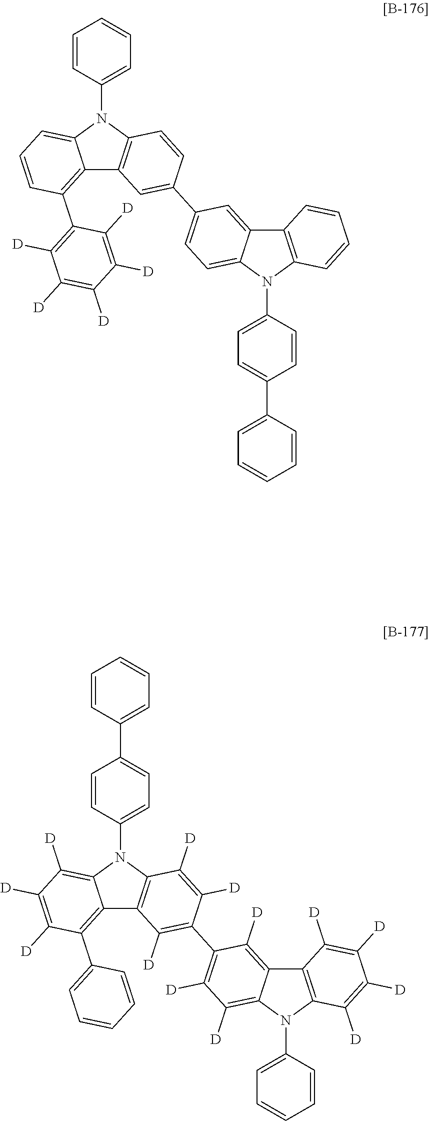 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

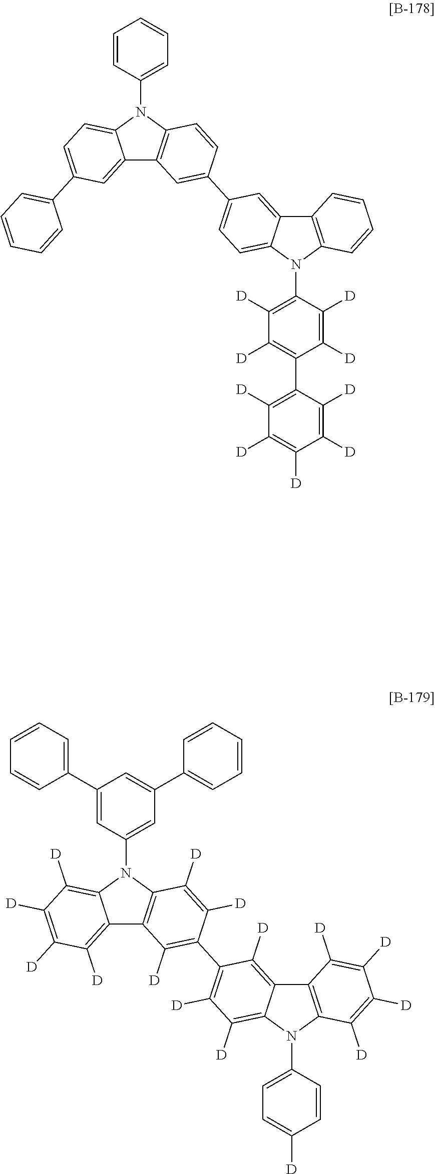 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H] ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

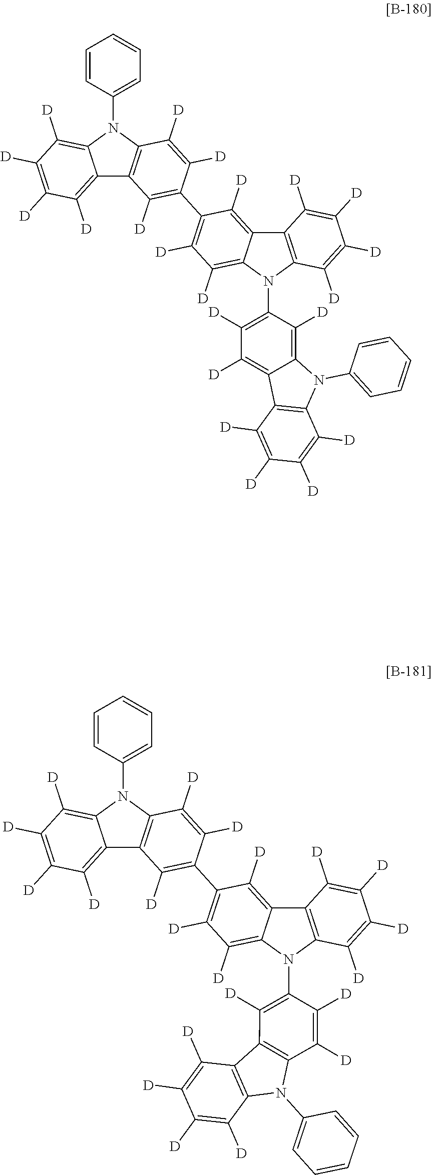 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

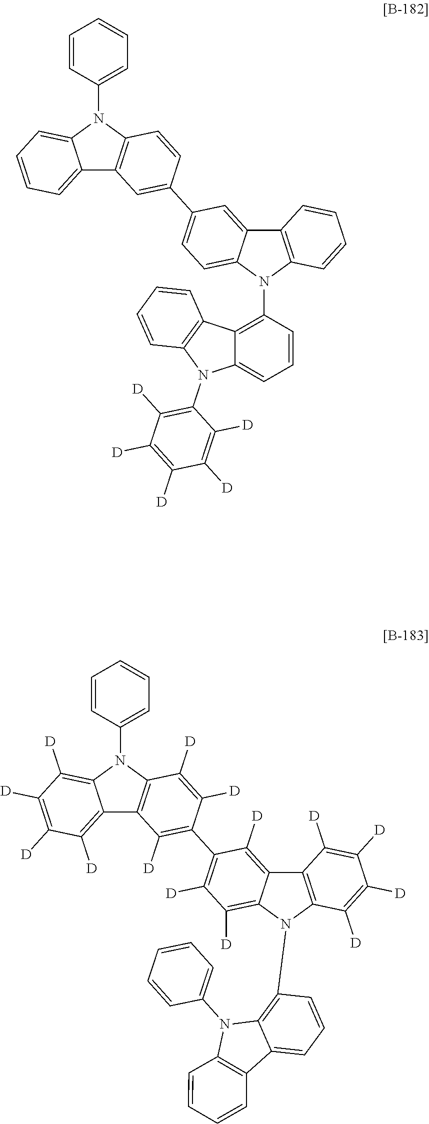 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

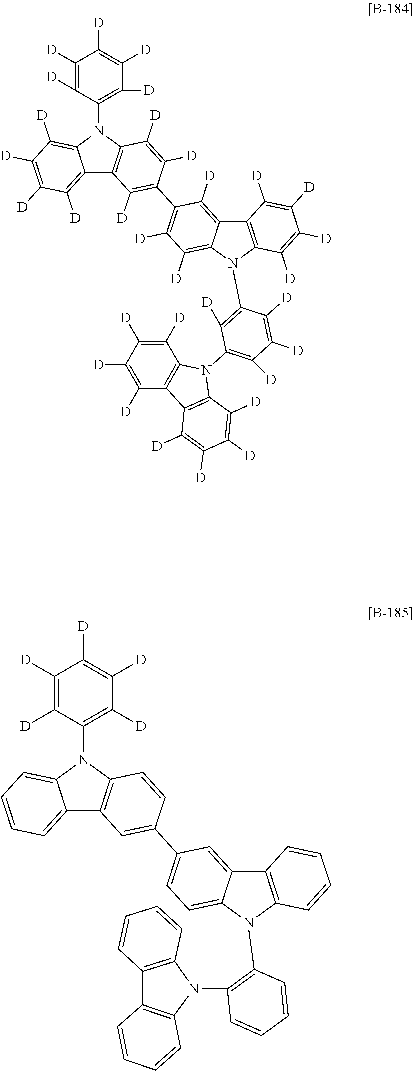 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

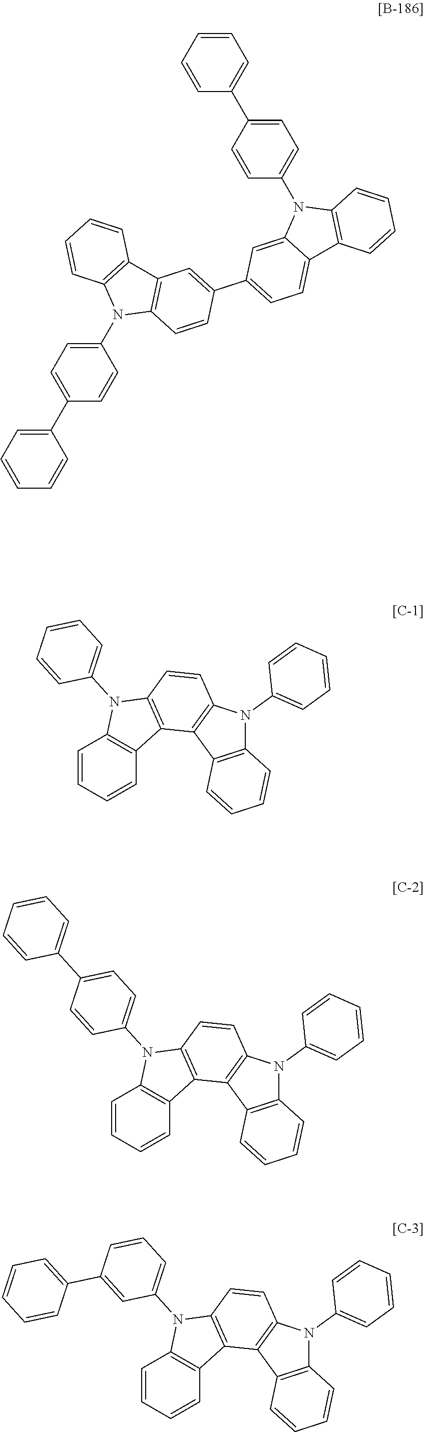 [SMILES: c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1]

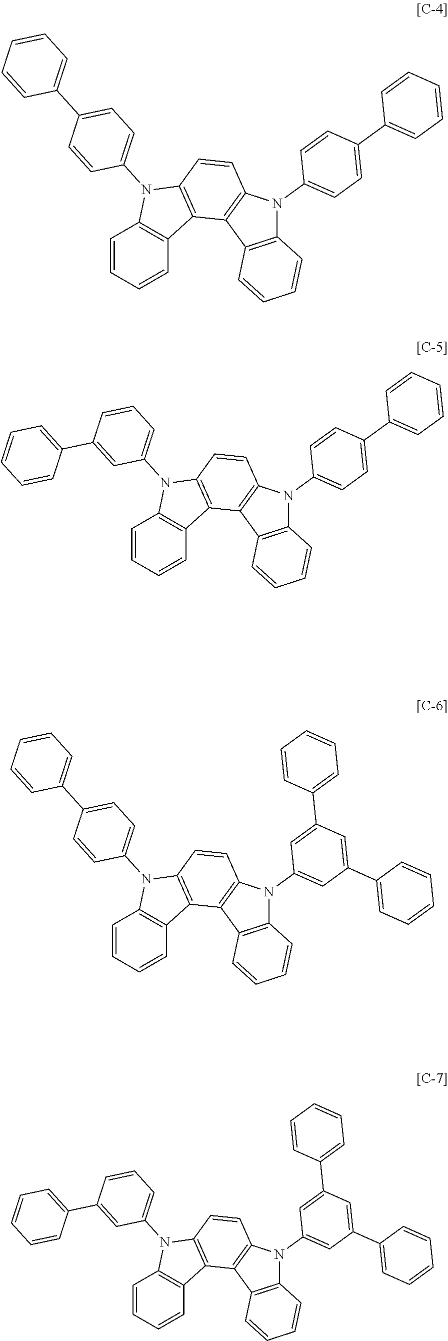 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1]

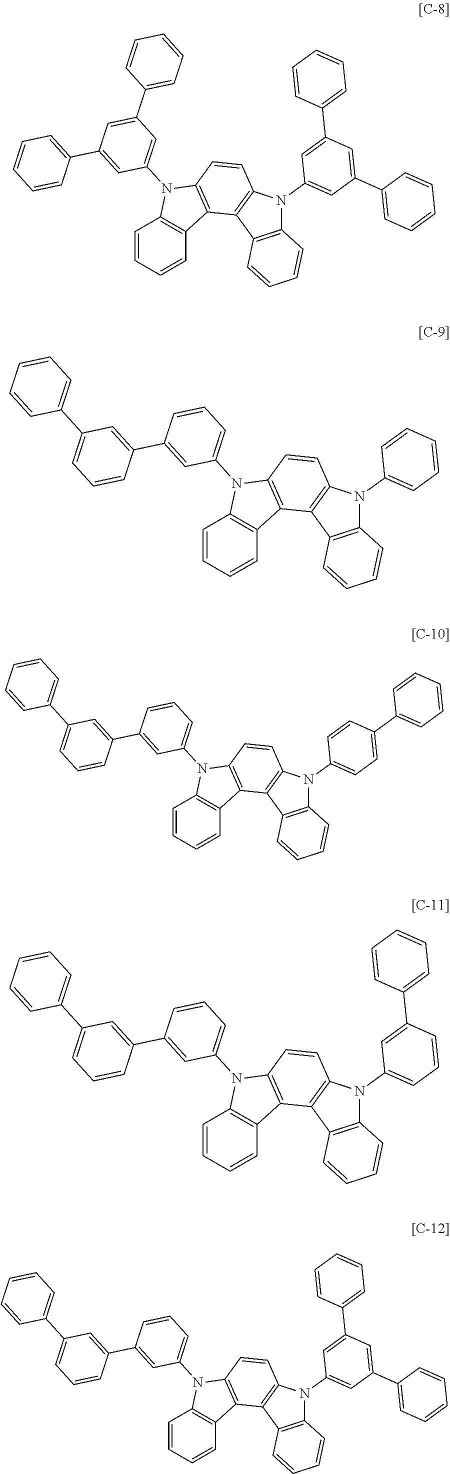 [SMILES: c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

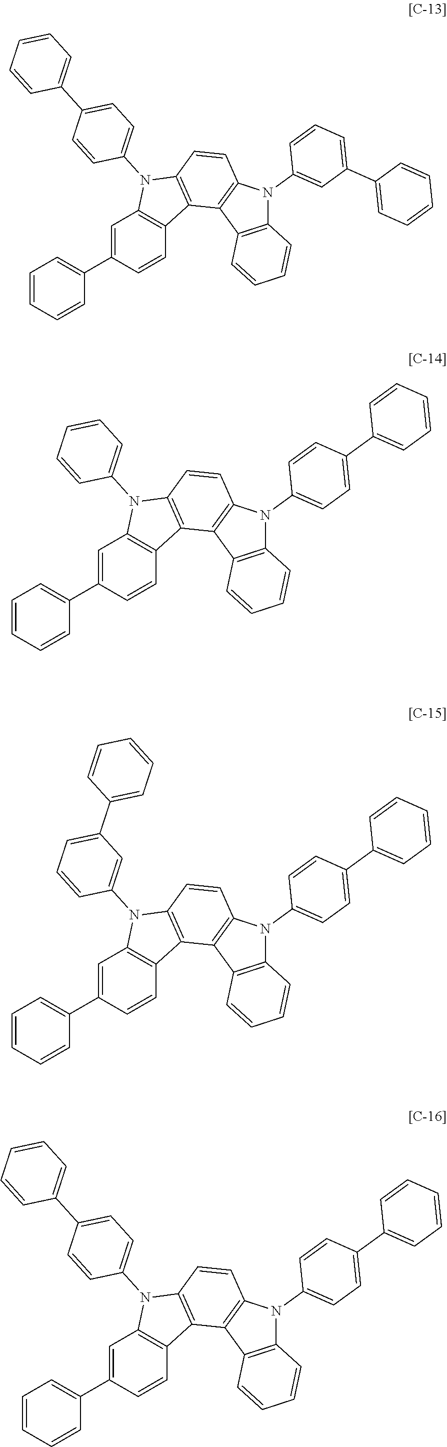 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

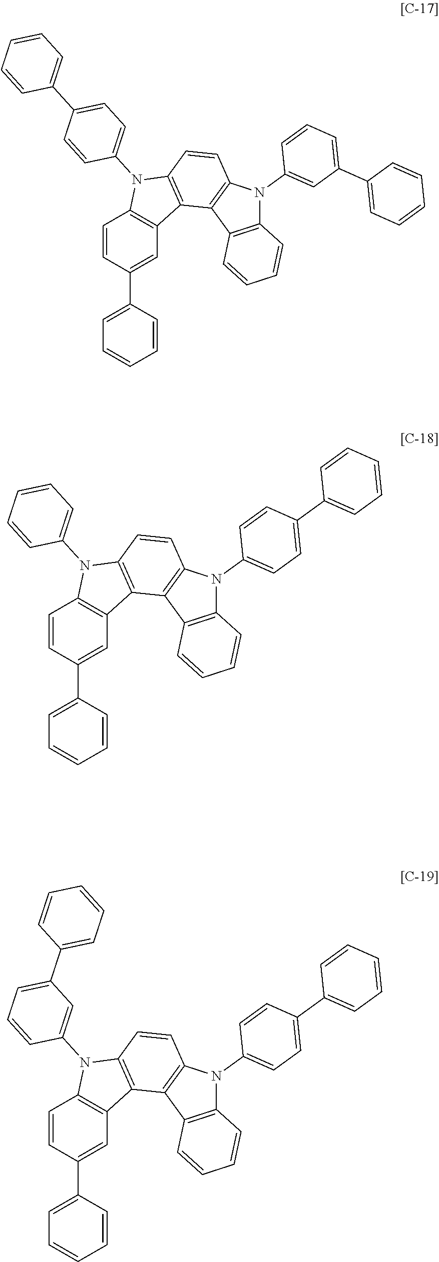 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1]

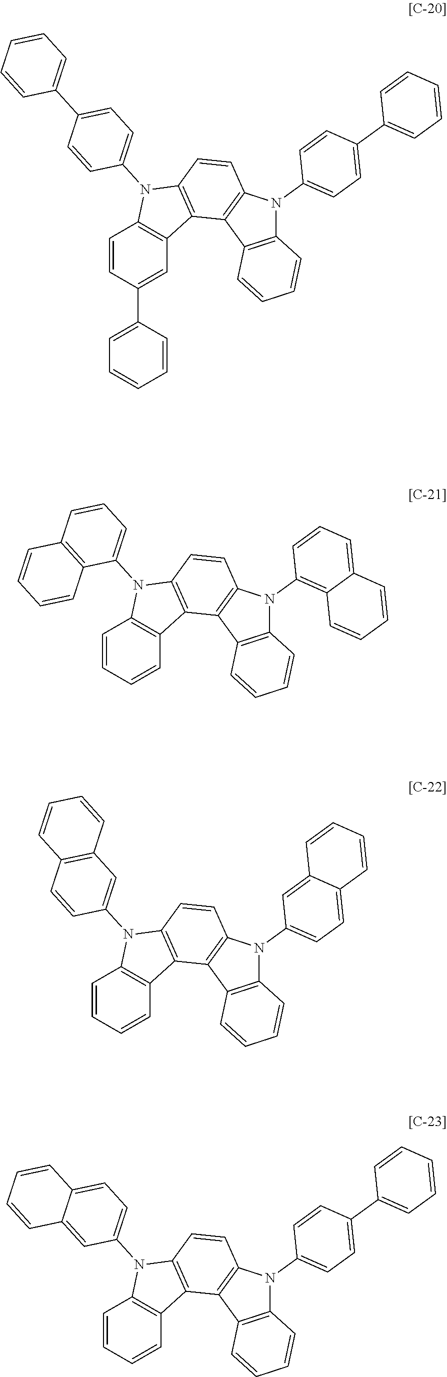 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1]

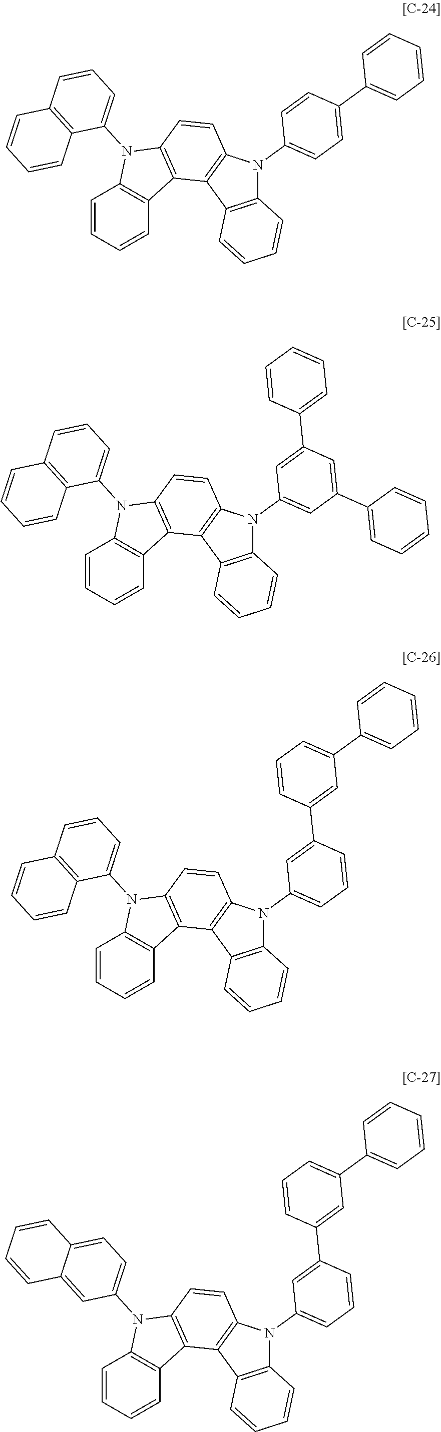 [SMILES: c1ccc(-c2ccc(-n3c4ccccc4c4c5c6ccccc6n(-c6cccc7ccccc67)c5ccc43)cc2)cc1 ; c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

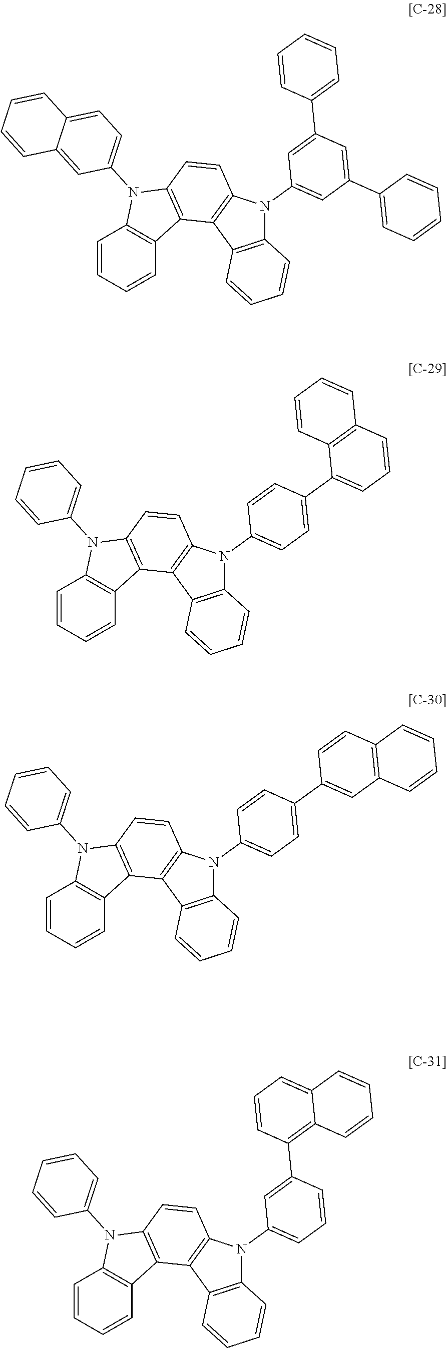 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1]

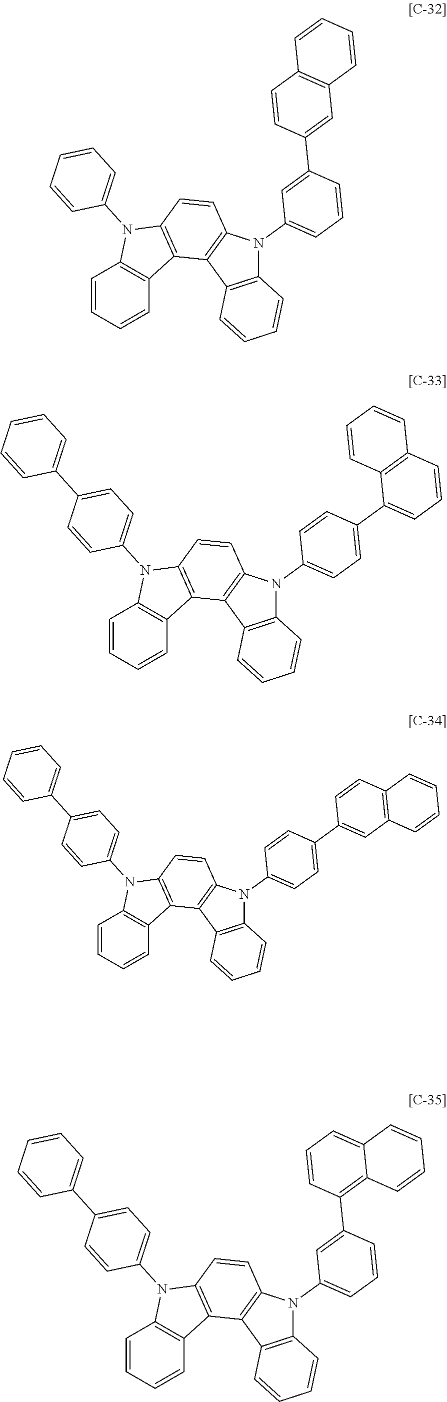 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1]

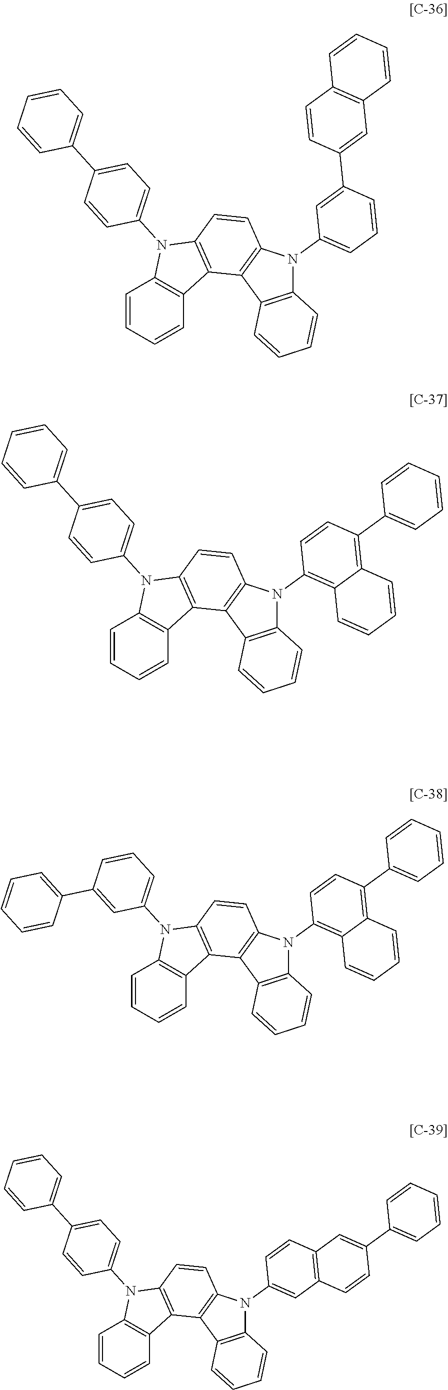 [SMILES: c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1]

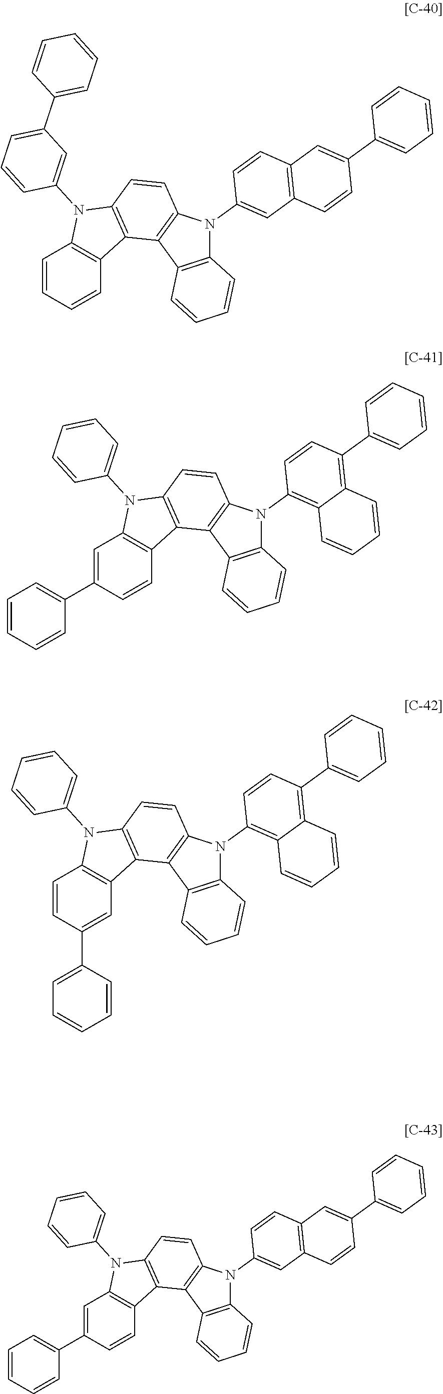 [SMILES: c1ccc(-c2cccc(-n3c4ccccc4c4c5c6ccccc6n(-c6ccc7cc(-c8ccccc8)ccc7c6)c5ccc43)c2)cc1 ; c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1]

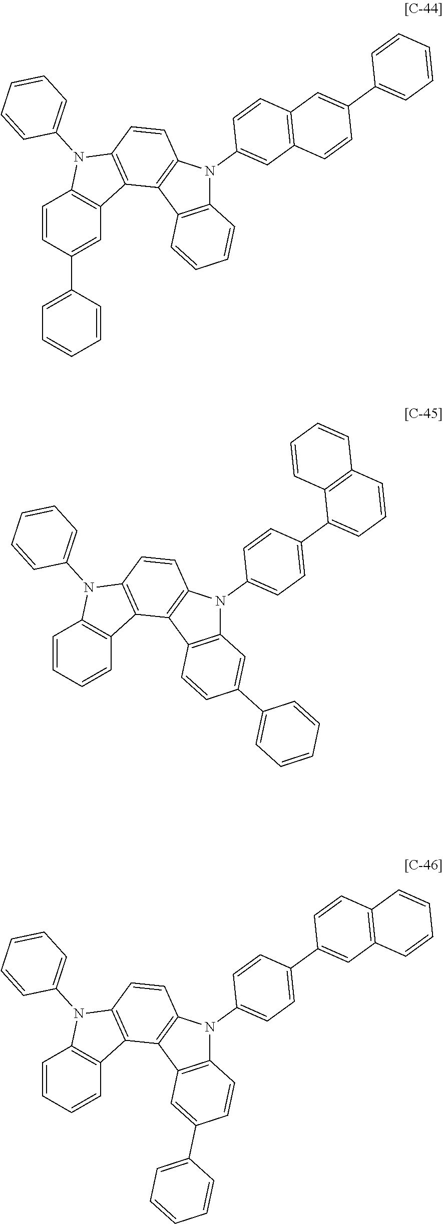 [SMILES: c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1]

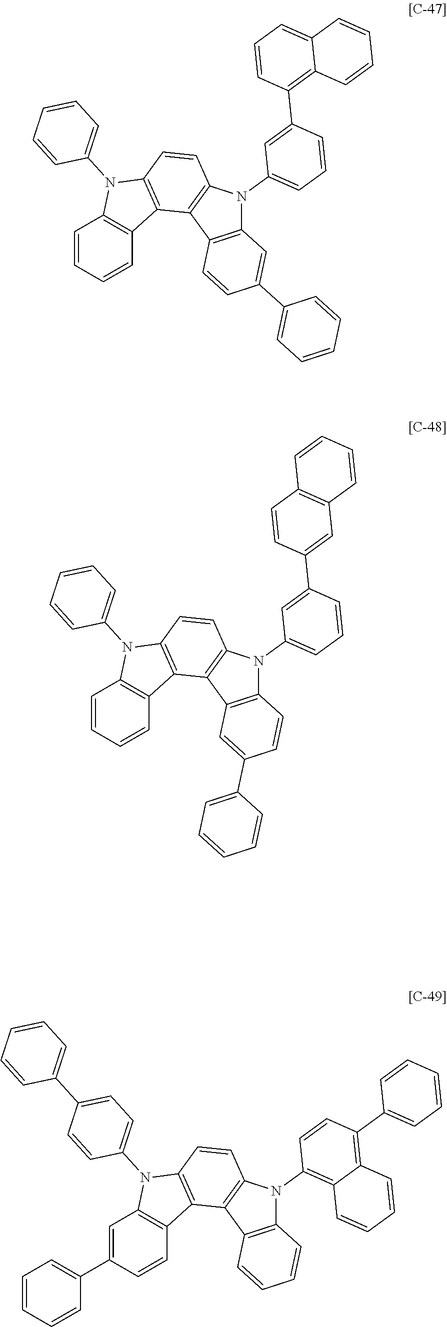 [SMILES: c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1]

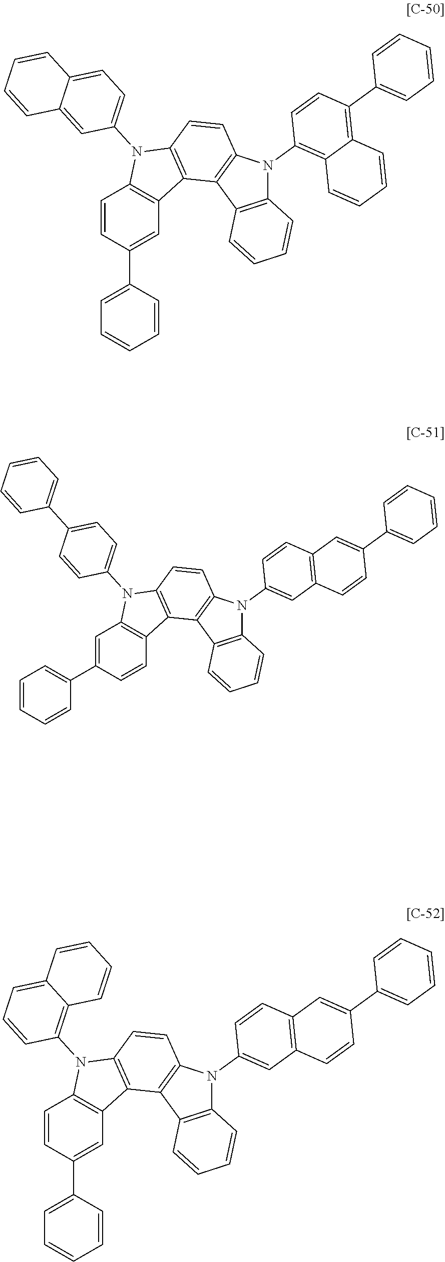 [SMILES: c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1]

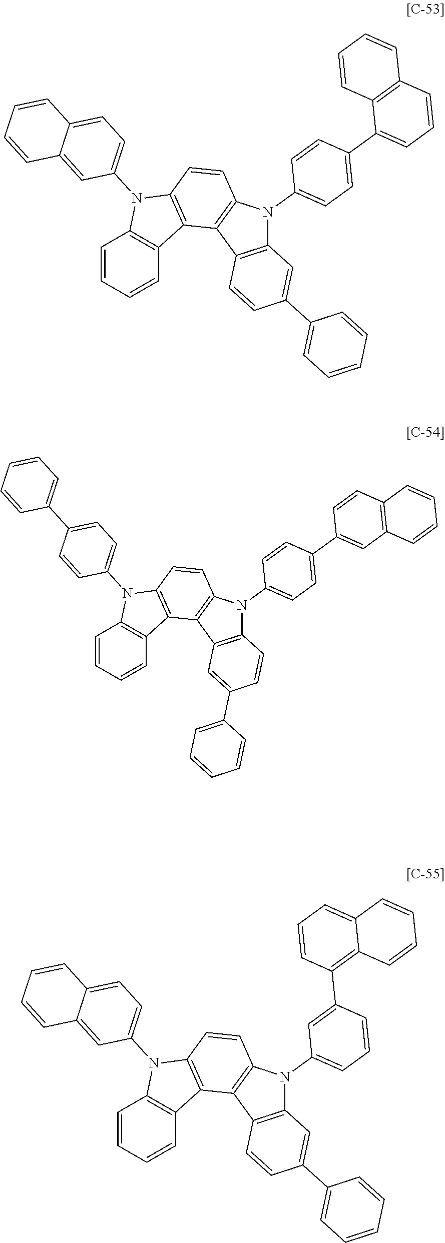 [SMILES: c1ccc(-c2ccc3c4c5c6ccccc6n(-c6ccc7ccccc7c6)c5ccc4n(-c4ccc(-c5cccc6ccccc56)cc4)c3c2)cc1 ; c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1]

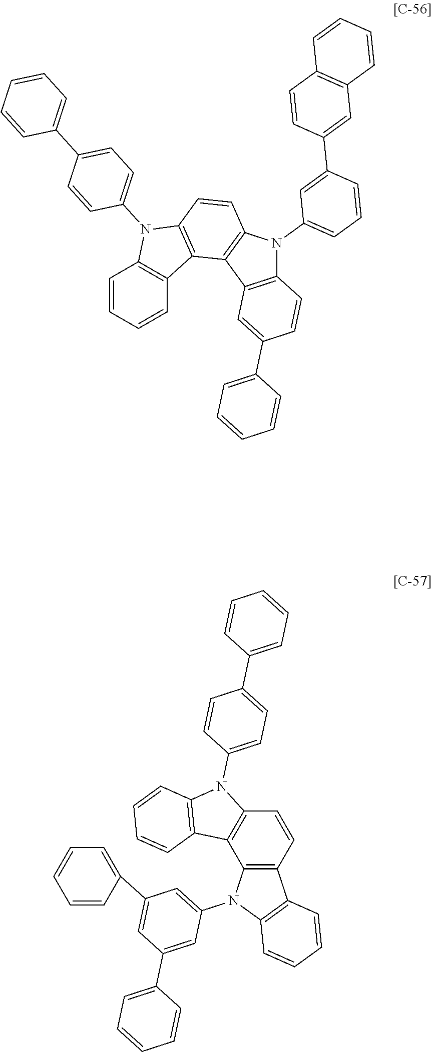 [SMILES: c1ccc(-c2ccc(-n3c4ccccc4c4c5c6cc(-c7ccccc7)ccc6n(-c6cccc(-c7ccc8ccccc8c7)c6)c5ccc43)cc2)cc1 ; c1ccc(-c2ccc(-n3c4ccccc4c4c3ccc3c5ccccc5n(-c5cc(-c6ccccc6)cc(-c6ccccc6)c5)c34)cc2)cc1]

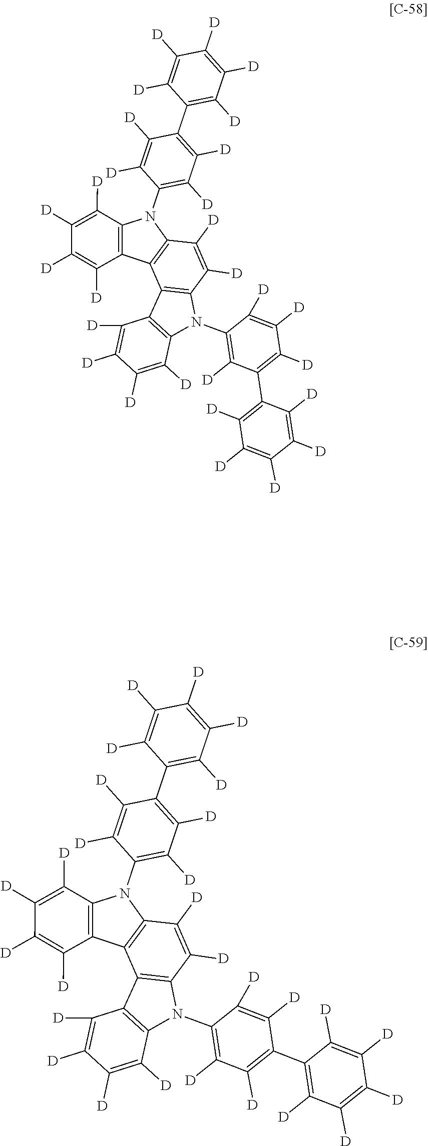 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

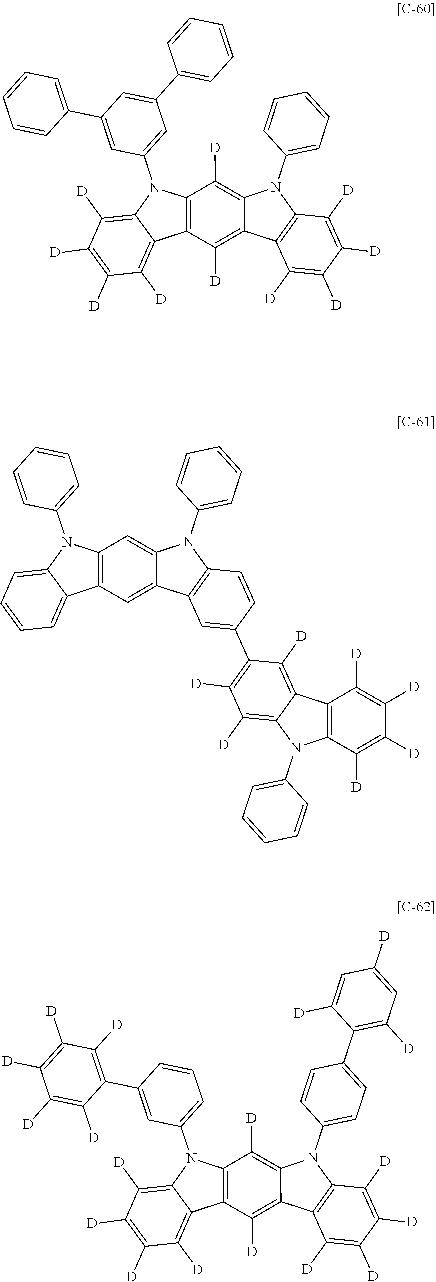 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

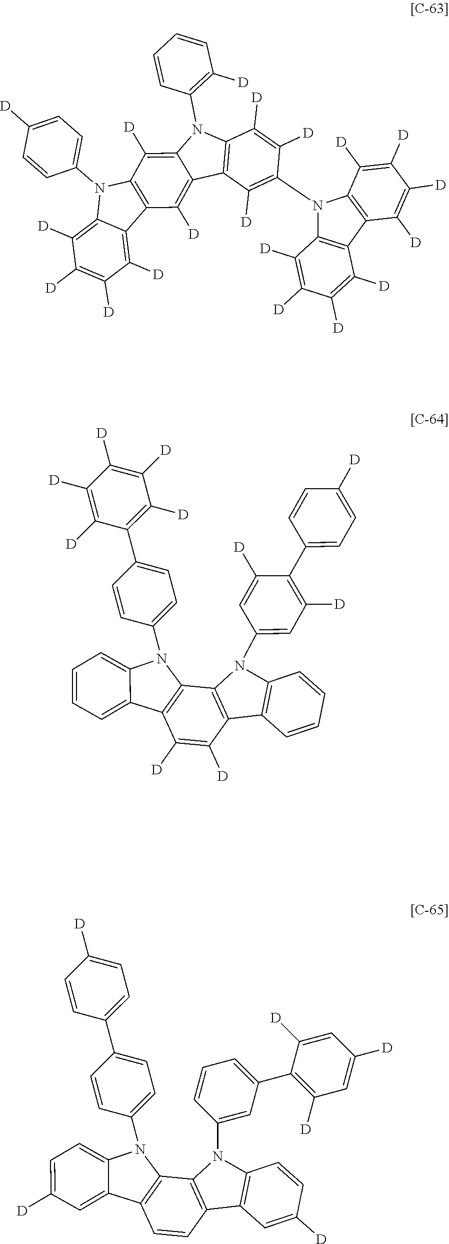 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1 ; [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

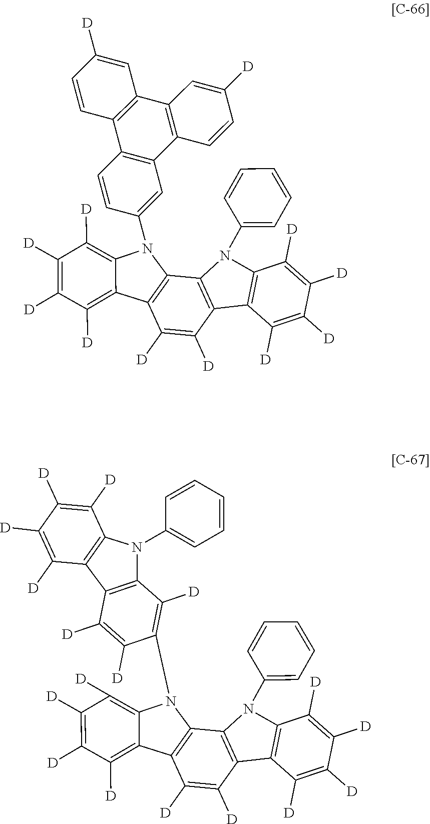 [SMILES: [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1 ; [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1]

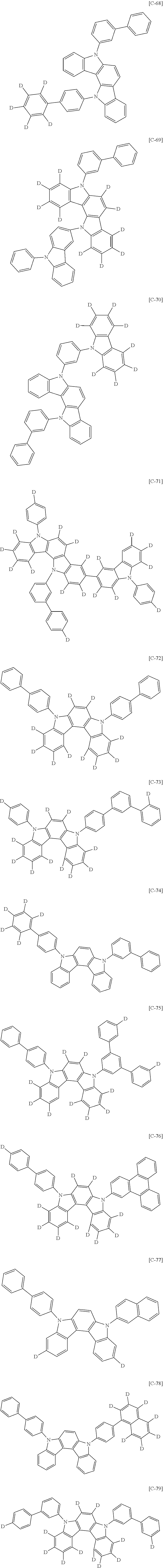

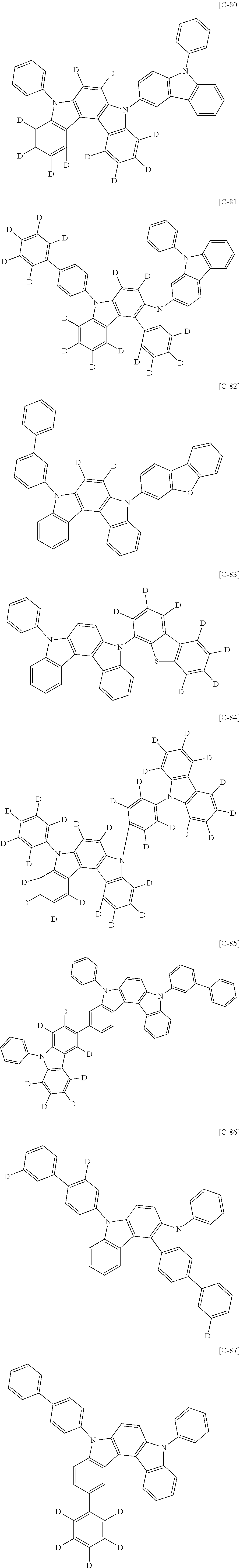

The third compound having buffer characteristics has a structure in which triphenylene is substituted with 9-carbazole and is represented by Chemical Formula 5.

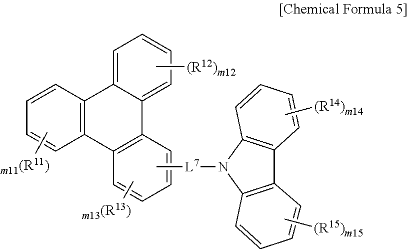

In Chemical Formula 5,
R¹¹ to R¹⁵ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
L⁷s are each independently a single bond or a substituted or unsubstituted C6 to C20 arylene group,
m11, m12, m14, and m15 are each independently one of integers of 1 to 4, and
m13 is one of integers of 1 to 3.

When m11 is 2 or more, each R¹¹ may be the same or different.

When m12 is 2 or more, each R¹² may be the same or different.

When m13 is 2 or more, each R¹³ may be the same or different.

When m14 is 2 or more, each R¹⁴ may be the same or different.

When m15 is 2 or more, each R¹⁵ may be the same or different.

Specifically, L⁷ may be a single bond, a substituted or unsubstituted phenylene group, a substituted or unsubstituted biphenylene group, or a substituted or unsubstituted terphenylene group.

More specifically, L⁷ may be a single bond, a substituted or unsubstituted phenylene group including at least one of a meta-phenylene group and a para-phenylene group, a substituted or unsubstituted biphenylene group including at least one of a meta-phenylene group and a para-phenylene group, or a terphenylene group including at least one of a meta-phenylene group and a para-phenylene group.

For example, L⁷ may be a single bond or may be selected from the linking groups listed in Group II.

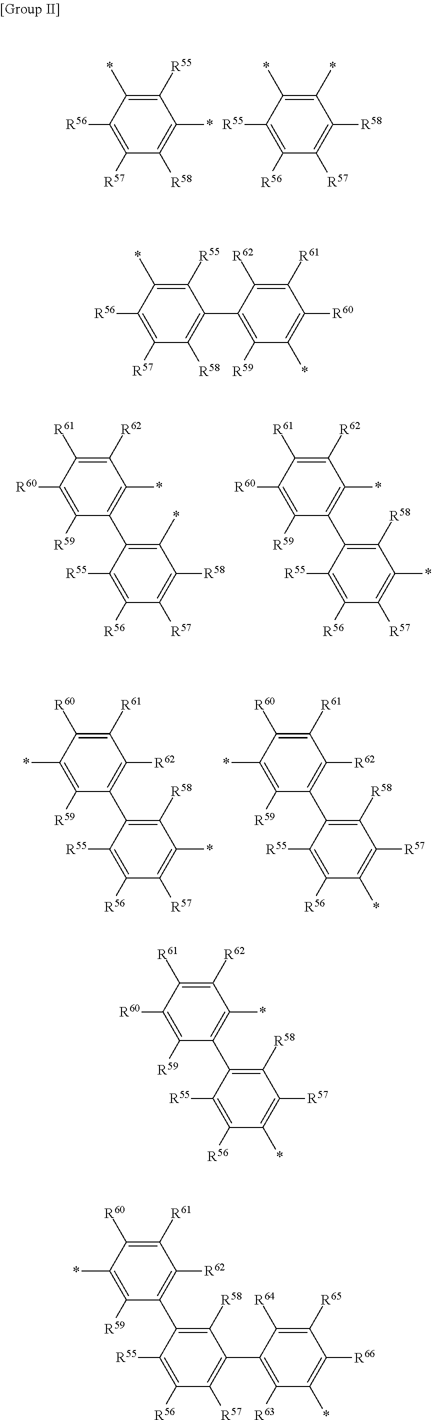

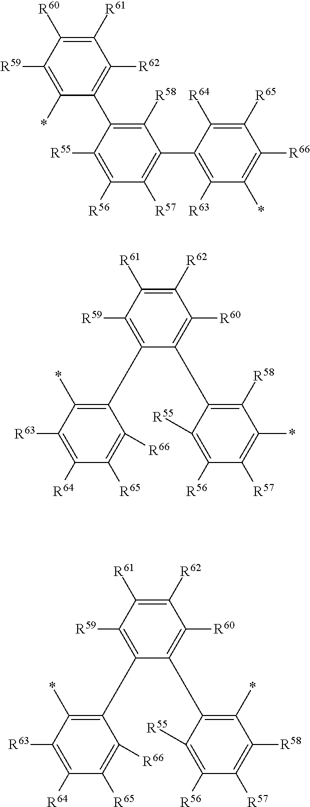

In Group II,
R⁵⁵ to R⁶⁶ are each independently hydrogen, deuterium, a C1 to C10 alkyl group, a substituted or unsubstituted C6 to C12 aryl group, a halogen, a cyano group, or a combination thereof, and
* is a linking point.

For example, it may be represented by any one of Chemical Formula 5-I to Chemical Formula 5-III.

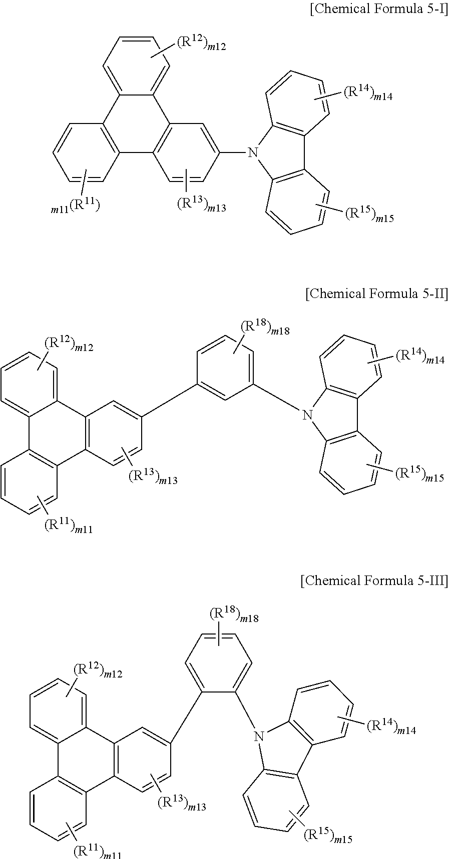

In Chemical Formula 5-I to Chemical Formula 5-III, R¹¹ to R¹⁵, R¹⁸, and m11 to m15 are the same as described above, and
m18 is one of integers of 1 to 4.

When m18 is 2 or more, each R¹⁸ may be the same or different.

For example, the third compound may be represented by Chemical Formula 5-II.

Specifically, in Chemical Formula 5, R¹¹ to R¹³ may each independently be hydrogen, deuterium, a substituted or unsubstituted phenyl group or a substituted or unsubstituted biphenyl group.

Specifically, in Chemical Formula 5, R¹⁴ and R¹⁵ may each independently be hydrogen, deuterium, a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group.

The third compound may be, for example, one selected from the compounds listed in Group 3.

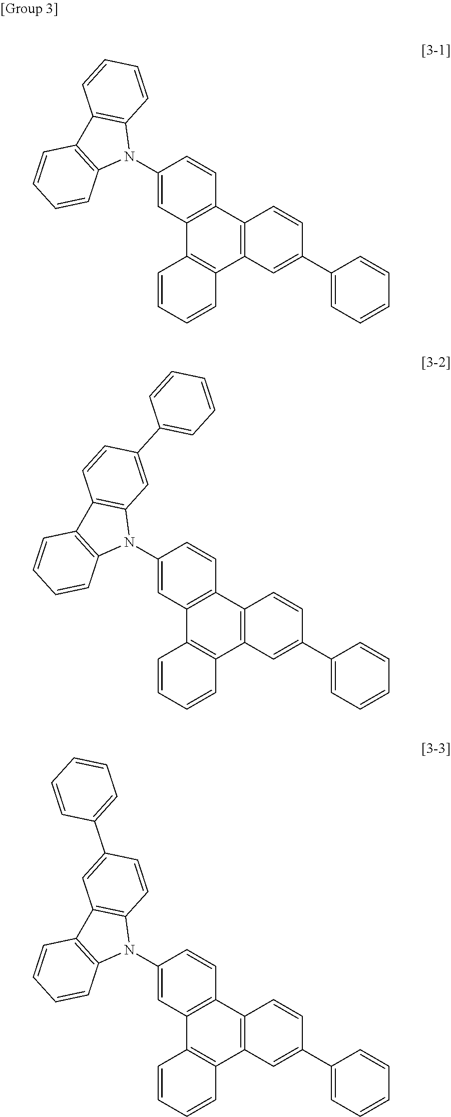 [SMILES: c1ccc(-c2ccc3c4ccc(-n5c6ccccc6c6ccccc65)cc4c4ccccc4c3c2)cc1 ; c1ccc(-c2ccc3c4ccc(-n5c6ccccc6c6ccc(-c7ccccc7)cc65)cc4c4ccccc4c3c2)cc1 ; c1ccc(-c2ccc3c4ccc(-n5c6ccccc6c6cc(-c7ccccc7)ccc65)cc4c4ccccc4c3c2)cc1]

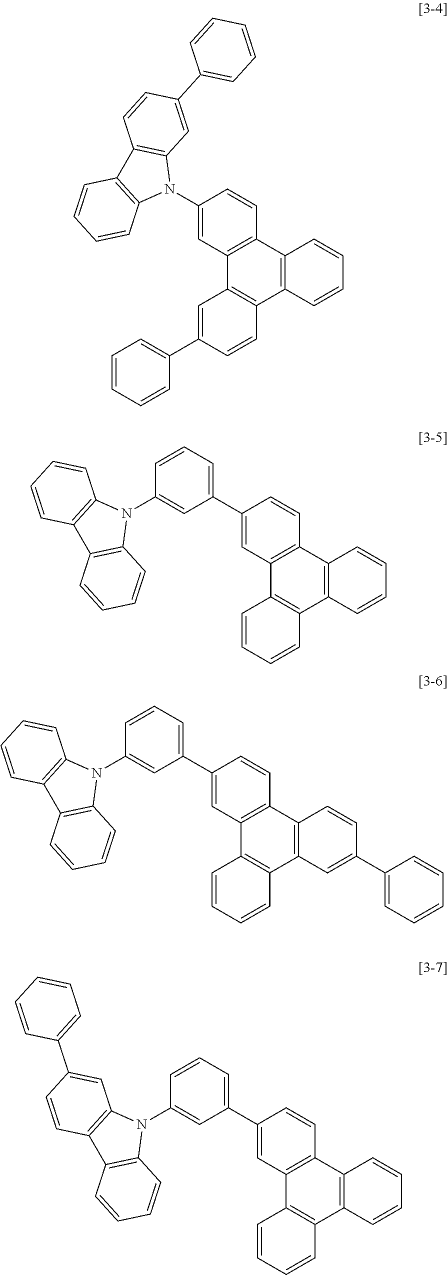 [SMILES: c1ccc(-c2ccc3c4ccccc4c4ccc(-n5c6ccccc6c6ccc(-c7ccccc7)cc65)cc4c3c2)cc1 ; c1cc(-c2ccc3c4ccccc4c4ccccc4c3c2)cc(-n2c3ccccc3c3ccccc32)c1 ; c1ccc(-c2ccc3c4ccc(-c5cccc(-n6c7ccccc7c7ccccc76)c5)cc4c4ccccc4c3c2)cc1 ; c1ccc(-c2ccc3c4ccccc4n(-c4cccc(-c5ccc6c7ccccc7c7ccccc7c6c5)c4)c3c2)cc1]

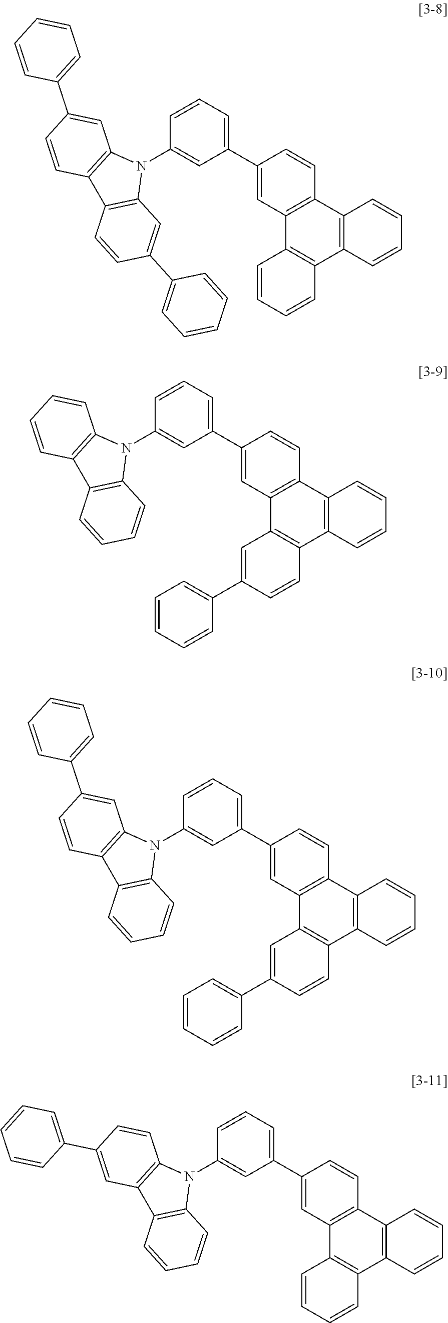

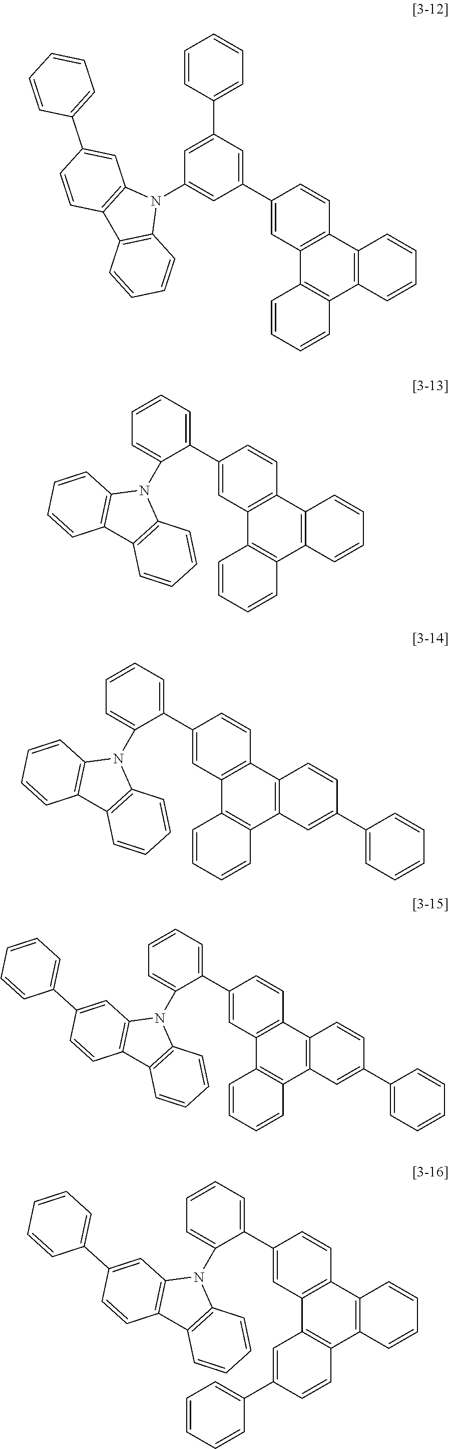 [SMILES: c1ccc(-c2cc(-c3ccc4c5ccccc5c5ccccc5c4c3)cc(-n3c4ccccc4c4ccc(-c5ccccc5)cc43)c2)cc1 ; c1ccc(-n2c3ccccc3c3ccccc32)c(-c2ccc3c4ccccc4c4ccccc4c3c2)c1 ; c1ccc(-c2ccc3c4ccc(-c5ccccc5-n5c6ccccc6c6ccccc65)cc4c4ccccc4c3c2)cc1 ; c1ccc(-c2ccc3c4ccc(-c5ccccc5-n5c6ccccc6c6ccc(-c7ccccc7)cc65)cc4c4ccccc4c3c2)cc1 ; c1ccc(-c2ccc3c4ccccc4c4ccc(-c5ccccc5-n5c6ccccc6c6ccc(-c7ccccc7)cc65)cc4c3c2)cc1]

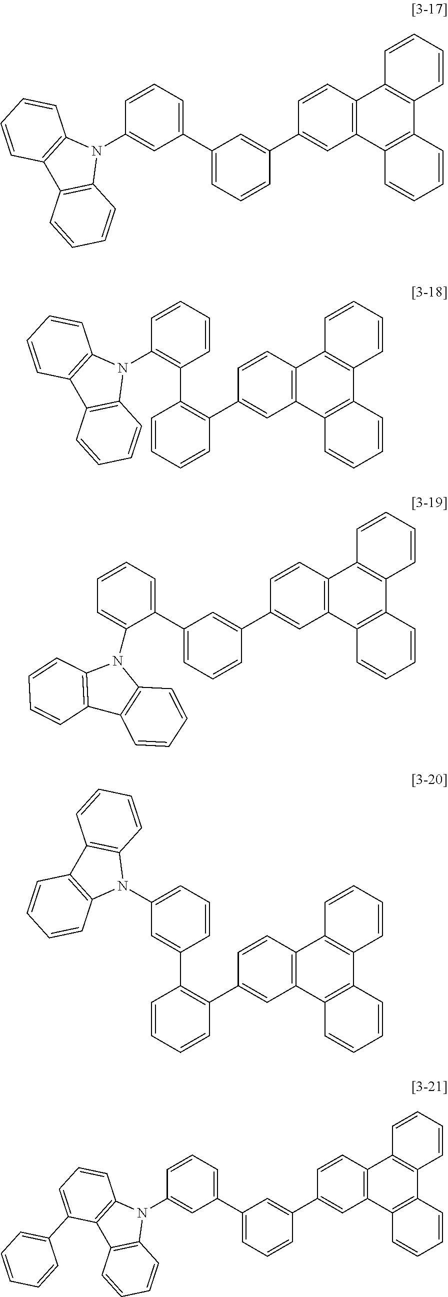 [SMILES: c1cc(-c2cccc(-n3c4ccccc4c4ccccc43)c2)cc(-c2ccc3c4ccccc4c4ccccc4c3c2)c1 ; c1ccc(-c2ccccc2-n2c3ccccc3c3ccccc32)c(-c2ccc3c4ccccc4c4ccccc4c3c2)c1 ; c1cc(-c2ccc3c4ccccc4c4ccccc4c3c2)cc(-c2ccccc2-n2c3ccccc3c3ccccc32)c1 ; c1cc(-c2ccccc2-c2ccc3c4ccccc4c4ccccc4c3c2)cc(-n2c3ccccc3c3ccccc32)c1 ; c1ccc(-c2cccc3c2c2ccccc2n3-c2cccc(-c3cccc(-c4ccc5c6ccccc6c6ccccc6c5c4)c3)c2)cc1]

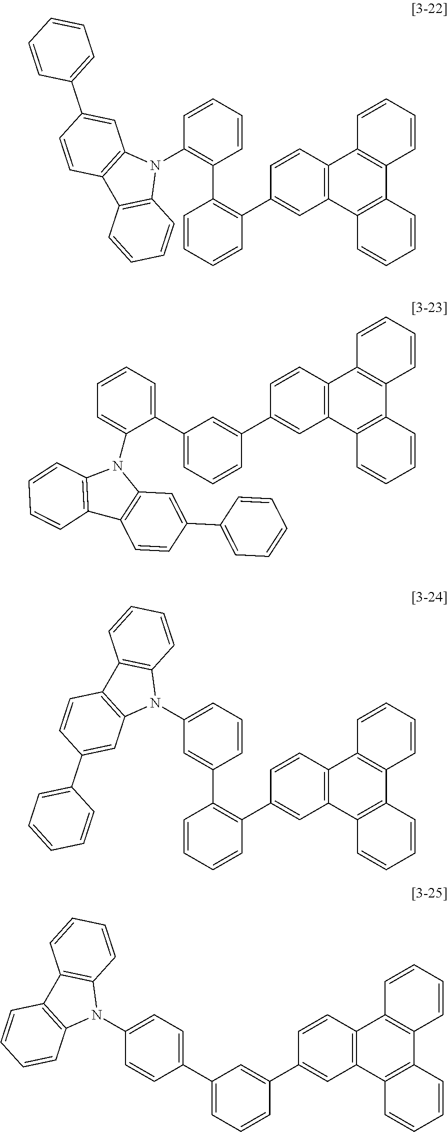 [SMILES: c1ccc(-c2ccc3c4ccccc4n(-c4ccccc4-c4ccccc4-c4ccc5c6ccccc6c6ccccc6c5c4)c3c2)cc1 ; c1ccc(-c2ccc3c4ccccc4n(-c4ccccc4-c4cccc(-c5ccc6c7ccccc7c7ccccc7c6c5)c4)c3c2)cc1 ; c1ccc(-c2ccc3c4ccccc4n(-c4cccc(-c5ccccc5-c5ccc6c7ccccc7c7ccccc7c6c5)c4)c3c2)cc1 ; c1cc(-c2ccc(-n3c4ccccc4c4ccccc43)cc2)cc(-c2ccc3c4ccccc4c4ccccc4c3c2)c1]

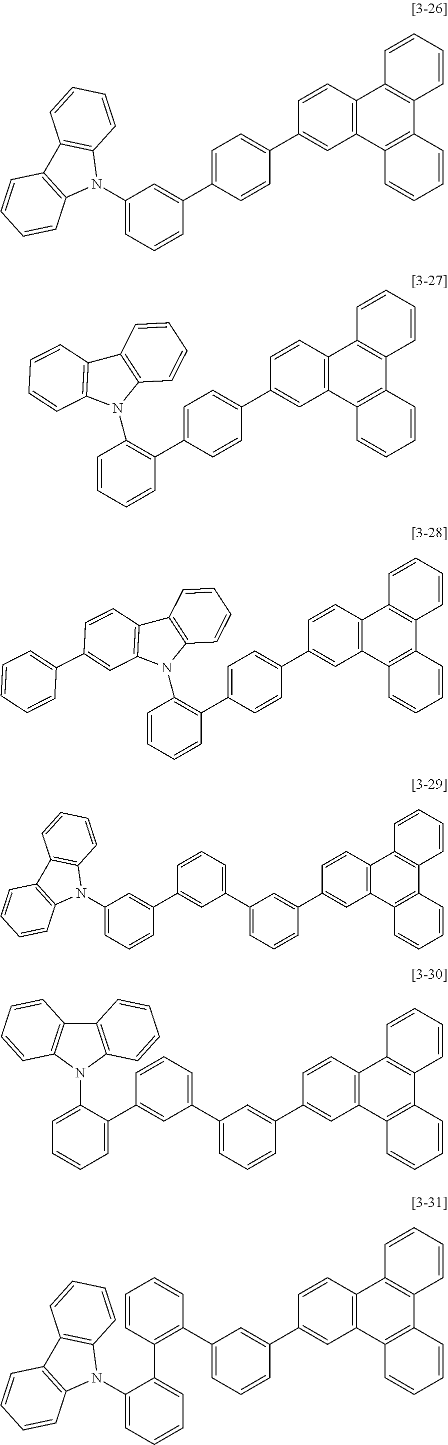 [SMILES: c1cc(-c2ccc(-c3ccc4c5ccccc5c5ccccc5c4c3)cc2)cc(-n2c3ccccc3c3ccccc32)c1 ; c1ccc(-n2c3ccccc3c3ccccc32)c(-c2ccc(-c3ccc4c5ccccc5c5ccccc5c4c3)cc2)c1 ; c1ccc(-c2ccc3c4ccccc4n(-c4ccccc4-c4ccc(-c5ccc6c7ccccc7c7ccccc7c6c5)cc4)c3c2)cc1 ; c1cc(-c2cccc(-c3ccc4c5ccccc5c5ccccc5c4c3)c2)cc(-c2cccc(-n3c4ccccc4c4ccccc43)c2)c1 ; c1cc(-c2cccc(-c3ccccc3-n3c4ccccc4c4ccccc43)c2)cc(-c2ccc3c4ccccc4c4ccccc4c3c2)c1 ; c1cc(-c2ccc3c4ccccc4c4ccccc4c3c2)cc(-c2ccccc2-c2ccccc2-n2c3ccccc3c3ccccc32)c1]

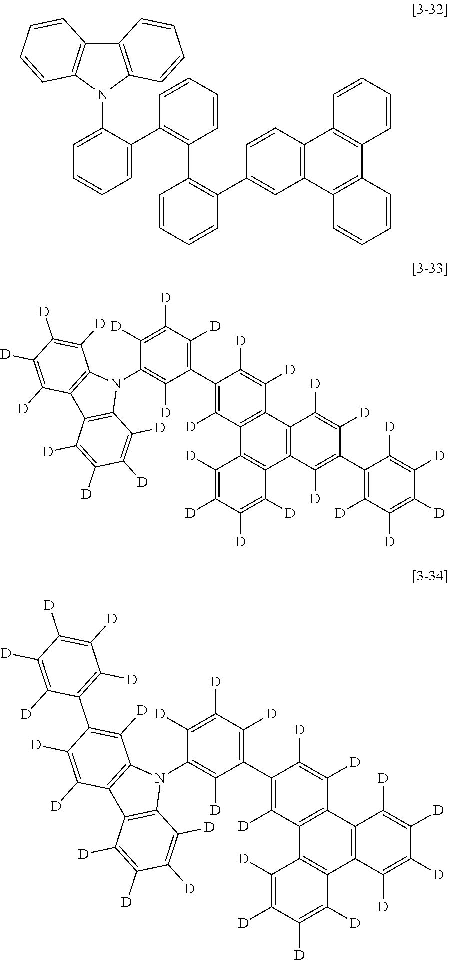

The first compound includes a nitrogen-containing 6-membered ring having high electron transport characteristics, and thus electrons may be stably and effectively transported to lower a driving voltage, to increase current efficiency, and to implement long life-span characteristics of a device.

The second compound has a structure including carbazole having high HOMO energy, and thus can effectively inject and transport holes, thereby contributing to improvement of device characteristics.

The third compound has a wide HOMO-LUMO band gap, thereby controlling the movement rate of holes and electrons of the first compound and the second compound, and thus hole trapping and exciton quenching may be prevented through relative movement of the light emitting layer region, which contributes to the improvement of the life-span characteristics of the device.

The three-host composition including the first compound, the second compound, and the third compound may achieve an optimum balance achieved by more finely adjusting electron/hole characteristics in the device stack compared with the composition, and may improve device characteristics greatly due to an appropriate balance of charges, compared with two-host composition such as a composition including the first compound and the second compound or a composition including the first compound and the third compound.

In addition, this three-host composition, compared with other three-host compositions with different combinations therefrom, may further improve life-span characteristics and efficiency characteristics by optimally adjusting balance of electron/hole characteristics.

In the most specific embodiment of the present invention, the first compound may be represented by Chemical Formula 1-A, the second compound may be represented by any one of Chemical Formulas 2-6, 2-8, and 3C, and the third compound may be represented by Chemical Formula 5-II.

This mixed composition of the first, second, and third compounds may be, for example, as a phosphorescent host included in a light emitting layer of an organic light emitting diode, which will be described later.

In the composition for an organic optoelectronic device, the first compound may be included in an amount of about 20 wt % to 50 wt % based on a total weight of the first compound, the second compound may be included in an amount of about 40 wt % to 60 wt % based on the total weight of the first compound, the second compound, and the third compound may be included in an amount of about 10 wt % to 30 wt % based on the total weight of the first compound, the second compound, and the third compound.

Within the above range, for example, the first compound for an organic optoelectronic device may be included in an amount of about 25 wt % to 45 wt % based on the total weight of the first compound for an organic optoelectronic device, the second compound for an organic optoelectronic device, and the third compound for an organic optoelectronic device included in weight, the second compound for an organic optoelectronic device may be included in an amount of about 45 wt % to 60 wt % based on the total weight of the first compound for an organic optoelectronic device, the second compound for an organic optoelectronic device, and the third compound for an organic optoelectronic device, and the third compound for an organic optoelectronic device may be included in an amount of about 10 wt % to 25 wt % based on the total weight of the first compound for an organic optoelectronic device, the second compound for an organic optoelectronic device, and the third compound for an organic optoelectronic device.

In addition, as a specific example, the first compound may be included in an amount of about 30 wt % to 40 wt % based on the total weight of the first compound, the second compound, and the third compound, the second compound may be included in an amount of about 45 wt % to 55 wt % based on the total weight of the first compound, the second compound, and the third compound, and the third compound may be included in an amount of about 10 wt % to 20 wt % based on the total weight of the first compound, the second compound, and the third compound.

As a more specific example, the composition for the organic optoelectronic device may include the first compound: the second compound: the third compound in a weight ratio of about 35:55:10, or about 32:48:20. Within the above range, the electron transport capability of the first compound, the hole transport capability of the second compound, and the buffering capability of the third compound are properly harmonized to improve efficiency and life-span of the device.

The composition for an organic optoelectronic device may further include one or more compounds in addition to the first compound, the second compound, and the third compound.

The composition for an organic optoelectronic device may further include a dopant. The dopant may be, for example, a phosphorescent dopant, such as a red, green, or blue phosphorescent dopant, and may be, for example, a green phosphorescent dopant.

The dopant is a material mixed with the first compound, second compound, and third compound in a trace amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L⁹MX² [Chemical Formula Z]

In Chemical Formula Z, M is a metal, and L⁹ and X² are the same or different and are a ligand to form a complex compound with M.

The M may be for example Ir, Pt, Os, Ti, Zr, Hf, Eu, Tb, Tm, Fe, Co, Ni, Ru, Rh, Pd, or a combination thereof and L⁹ and X² may be for example a bidentate ligand.

Examples of ligands represented by L⁹ and X² may be selected from the Chemical Formulas listed in Group A, but are not limited thereto.

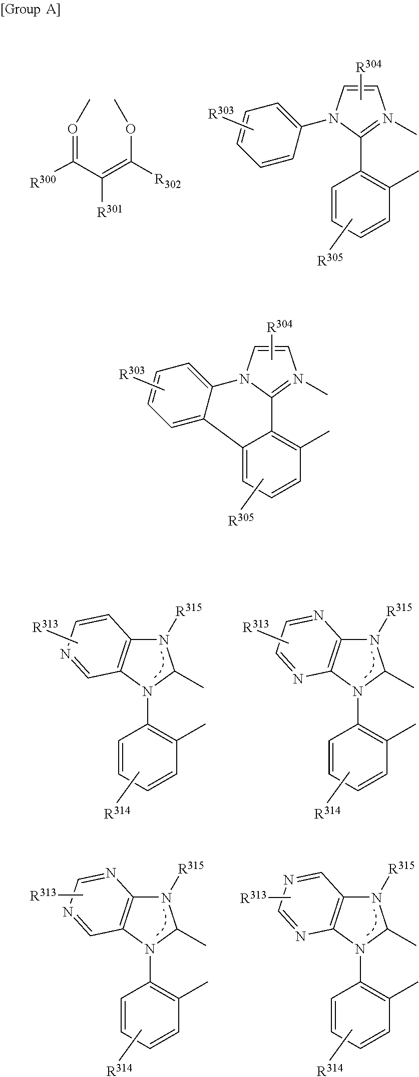

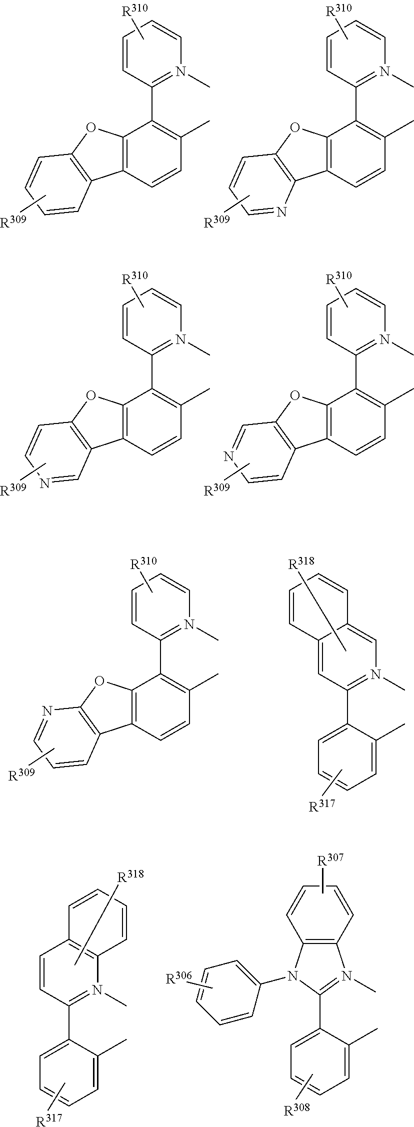

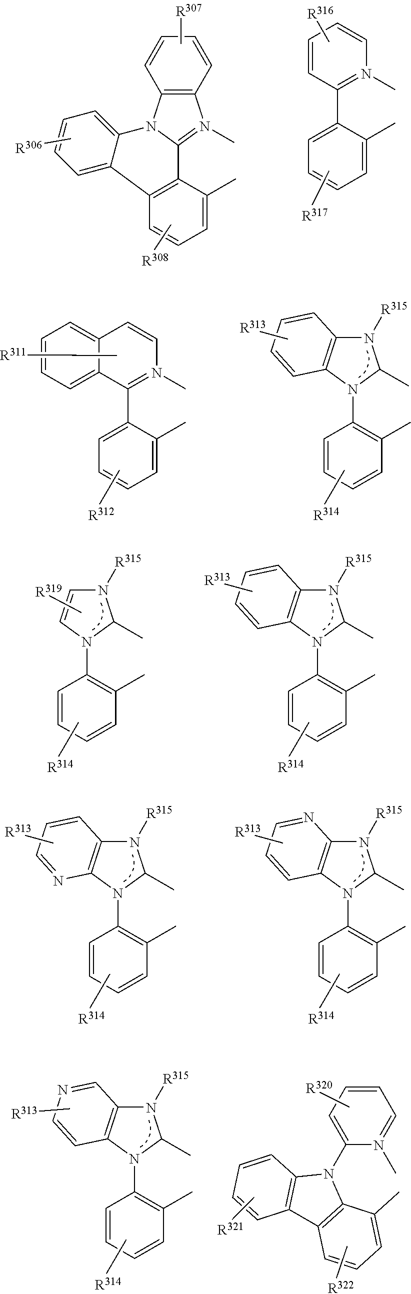

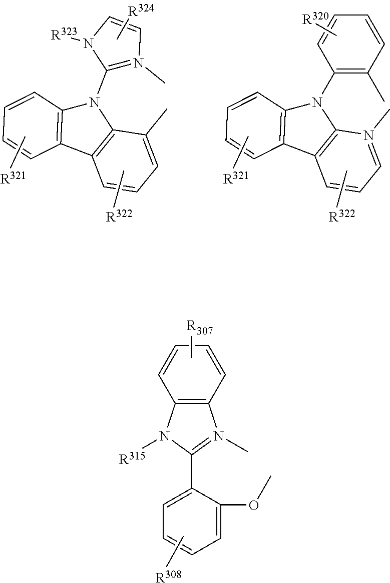

In Group A,
R³⁰⁰ to R³⁰² are each independently hydrogen, deuterium, a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

For example, a dopant represented by Chemical Formula IV may be further included.

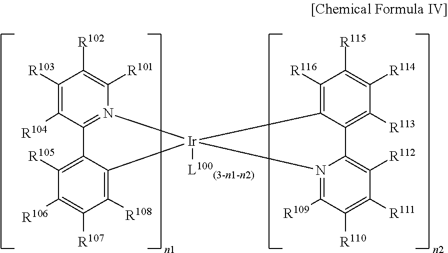

In Chemical Formula IV,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹⁰¹ to R¹¹⁶ is a functional group represented by Chemical Formula IV-1,
L¹⁰⁰ is a bidentate ligand of a single anion, which coordinates to iridium through the unshared electron pair of carbon or a heteroatom,
n1 and n2 are each independently any one of integers of 0 to 3, and n1+n2 is any one of integers of 1 to 3,

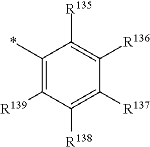

In Chemical Formula IV-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a portion linked to a carbon atom.

As an example, a dopant represented by Chemical Formula Z-1 may be included.

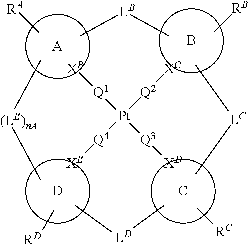

In Chemical Formula Z-1, rings A, B, C, and D independently represent a 5-membered or 6-membered carbocyclic or heterocyclic ring;
R^{A}, R^{B}, R^{C}, and R^{D} independently represent mono-, di-, tri-, or tetra-substitution, or unsubstitution;
L^{B}, L^{C}, and L^{D} are each independent selected from a direct bond, BR, NR, PR, O, S, Se, C═O, S═O, SO₂, CRR′, SiRR′, GeRR′, and a combination thereof,
when nA is 1, L^{E} is selected from a direct bond, BR, NR, PR, O, S, Se, C═O, S═O, SO₂, CRR′, SiRR′, GeRR′, and a combination thereof, when nA is 0, L^{E} does not exist; and
R^{A}, R^{B}, R^{C}, R^{D}, R, and R′ are each independently selected from hydrogen, deuterium, a halogen, alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof, any adjacent R^{A}R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

In an embodiment, the dopant may be a platinum complex, and may be represented by, for example, Chemical Formula V.

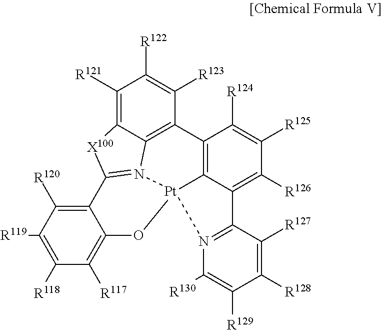

In Chemical Formula V,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group, and
at least one of R¹¹⁷ to R¹³¹ is —SiR¹³²R¹³³R¹³⁴ or a tert-butyl group.

The composition for an organic optoelectronic device may be formed by a dry film deposition method such as chemical vapor deposition.

Hereinafter, an organic optoelectronic device using the aforementioned composition for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Here, an organic light emitting diode as one example of an organic optoelectronic device is described referring to drawings.

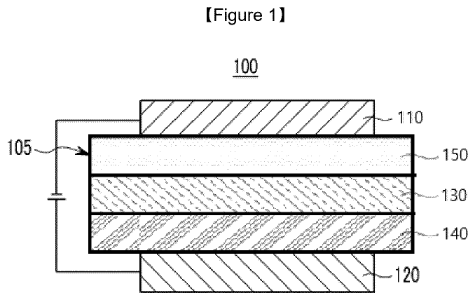

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof, a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may include a metal such as magnesium, calcium, sodium, potassium, titanium, indium, yttrium, lithium, gadolinium, aluminum, silver, tin, lead, cesium, barium, or an alloy thereof, a multilayer structure material such as LiF/Al, LiO₂/Al, LiF/Ca, and BaF₂/Ca, but is not limited thereto.

The organic layer 105 may include the aforementioned composition for an organic optoelectronic device.

The organic layer 105 may include a light emitting layer 130 and the light emitting layer 130 may include the aforementioned composition for an organic optoelectronic device.

The composition for an organic optoelectronic device further including a dopant may be, for example, a green light emitting composition.

The light emitting layer 130 may include, for example, the aforementioned composition for an organic optoelectronic device, as a phosphorescent host.

The organic layer may further include a charge transport region in addition to the light emitting layer.

The charge transport region may be, for example, a hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

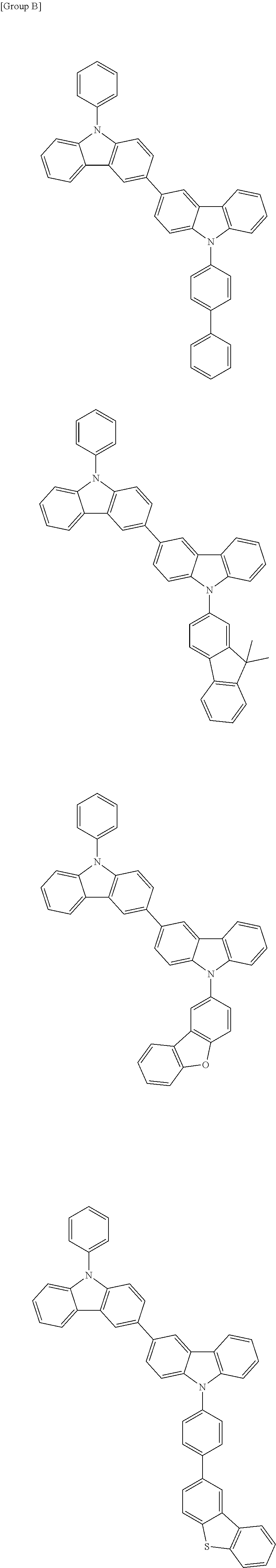 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

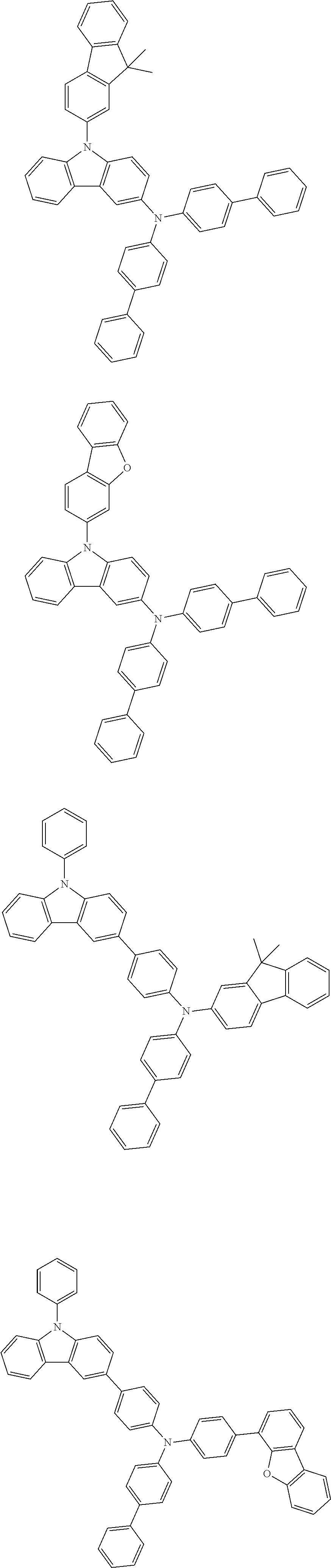 [SMILES: CC1(C)c2ccccc2-c2ccc(-n3c4ccccc4c4cc(N(c5ccc(-c6ccccc6)cc5)c5ccc(-c6ccccc6)cc5)ccc43)cc21 ; c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

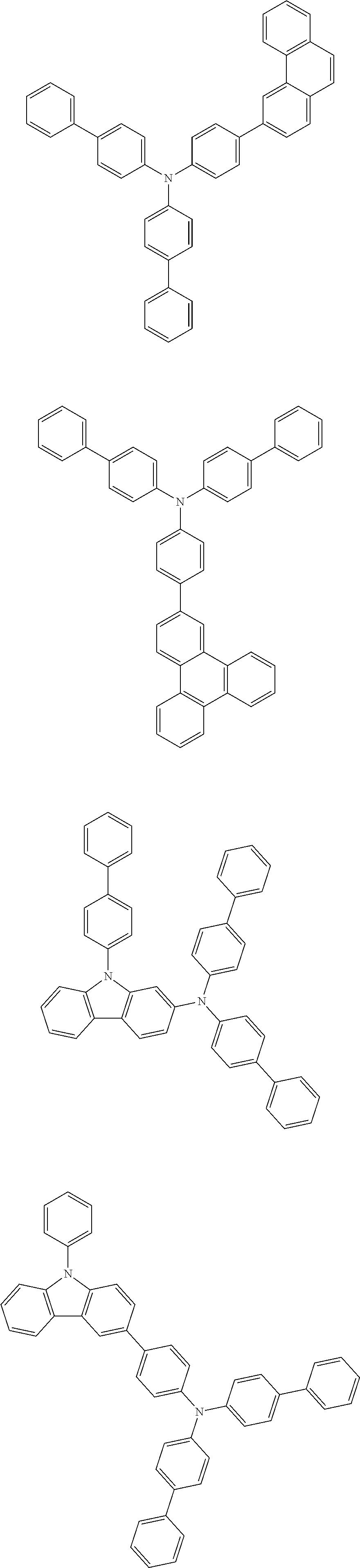 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

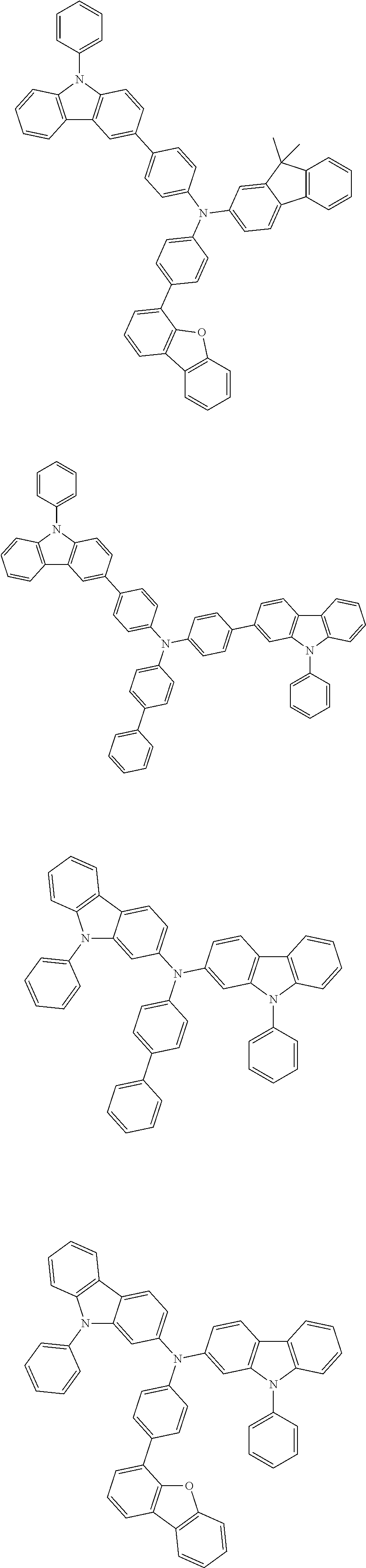 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

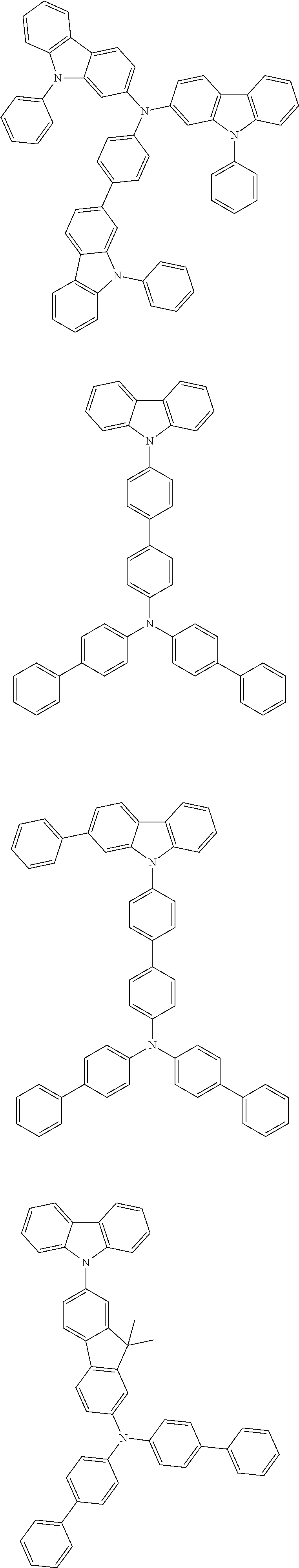 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

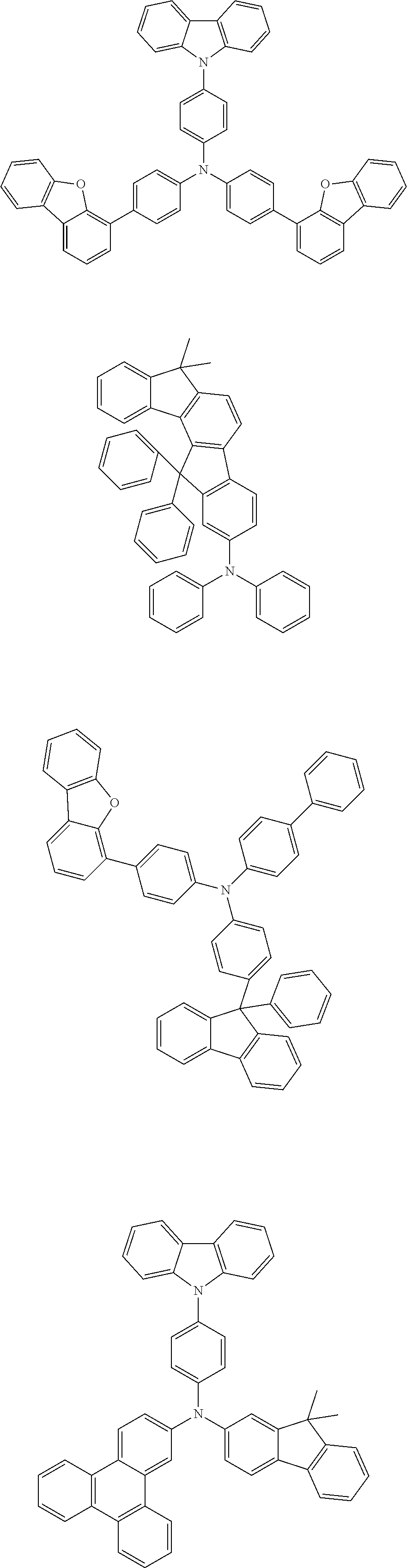 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21]

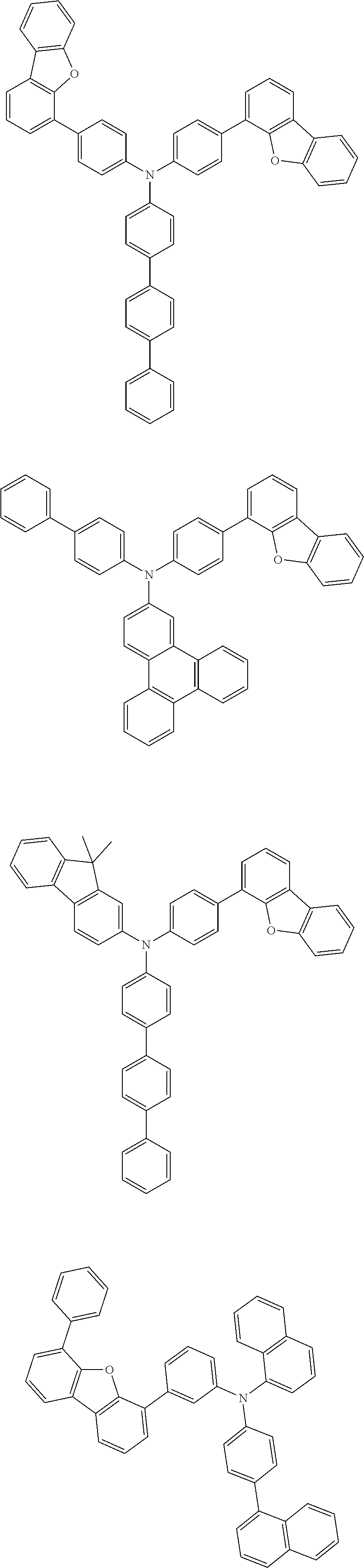 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

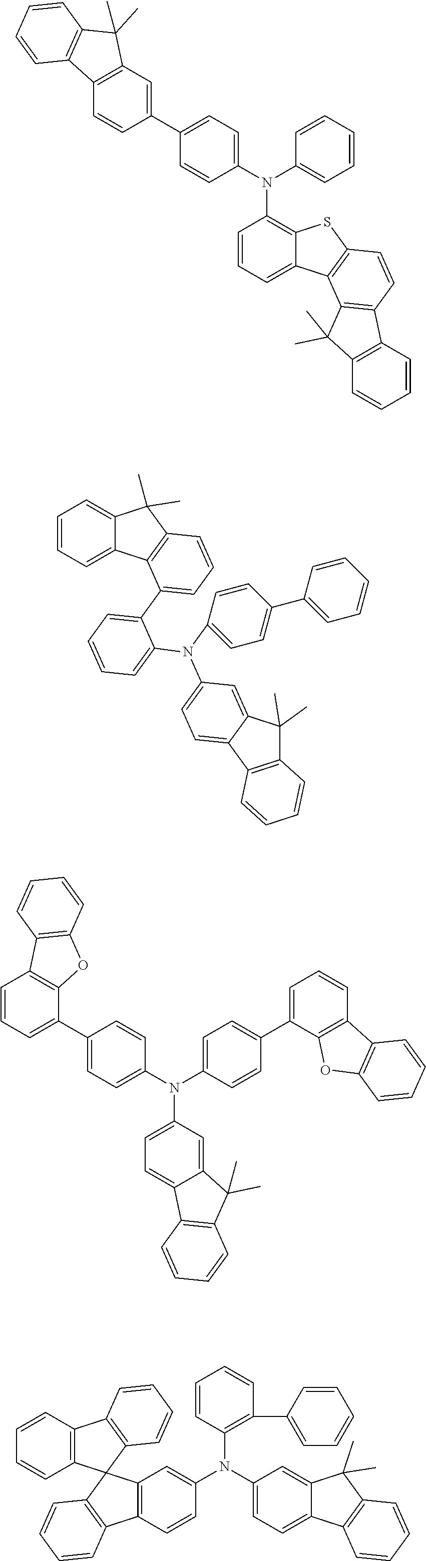 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

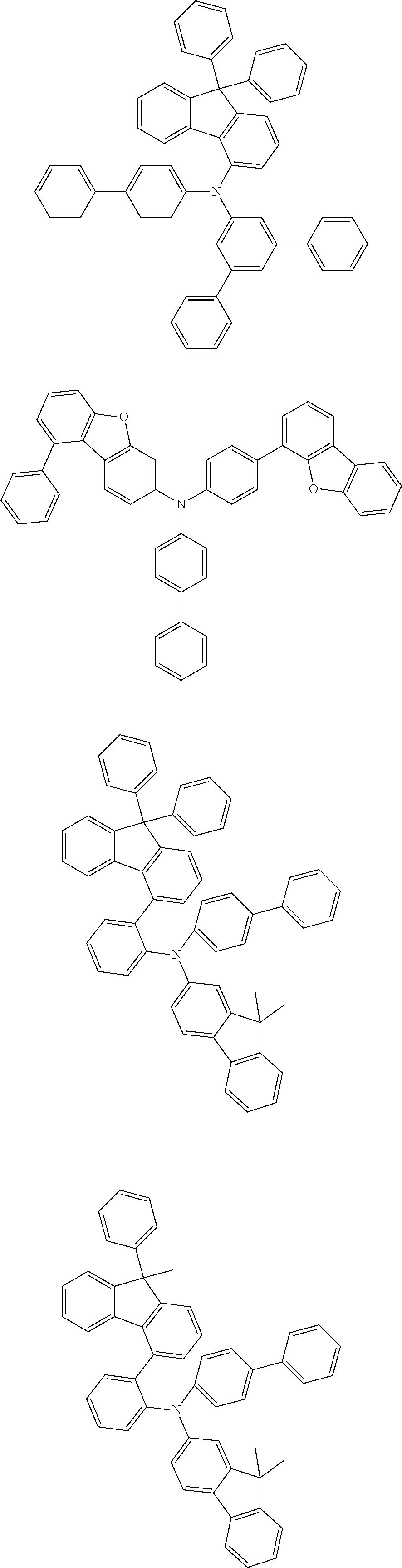 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

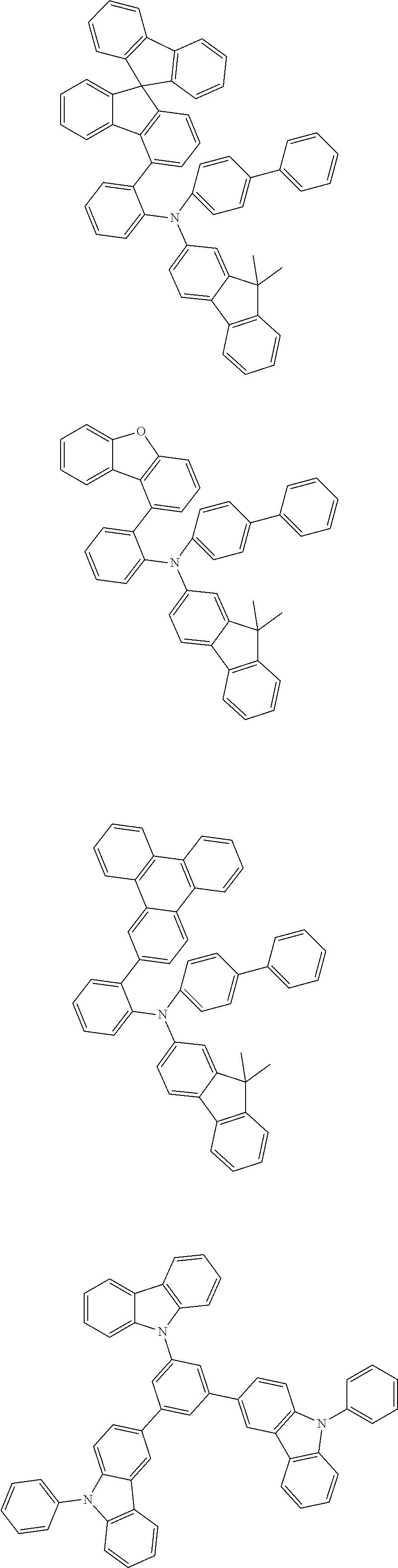 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1]

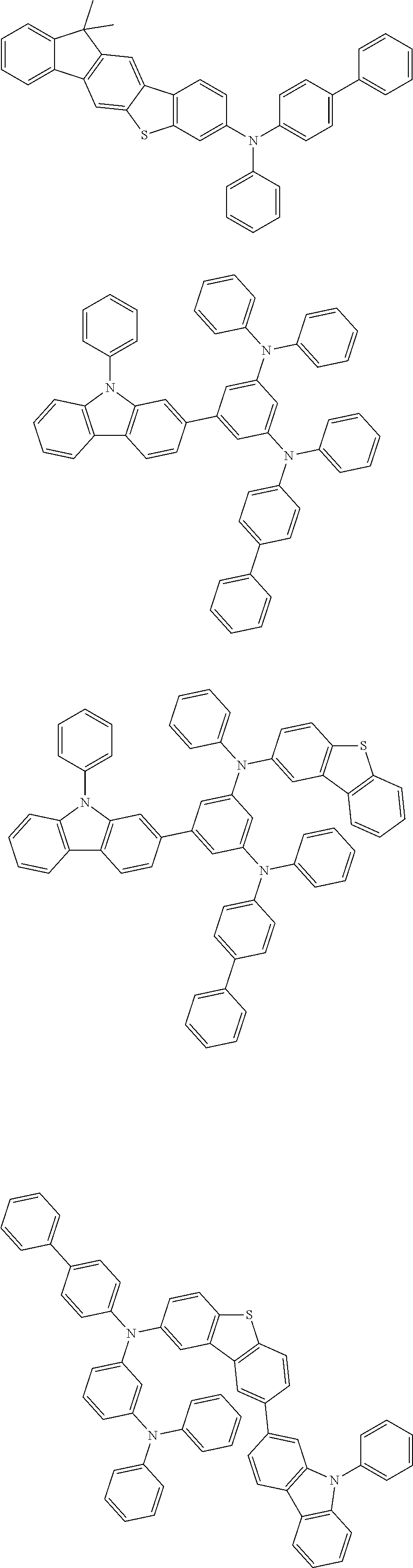 [SMILES: CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

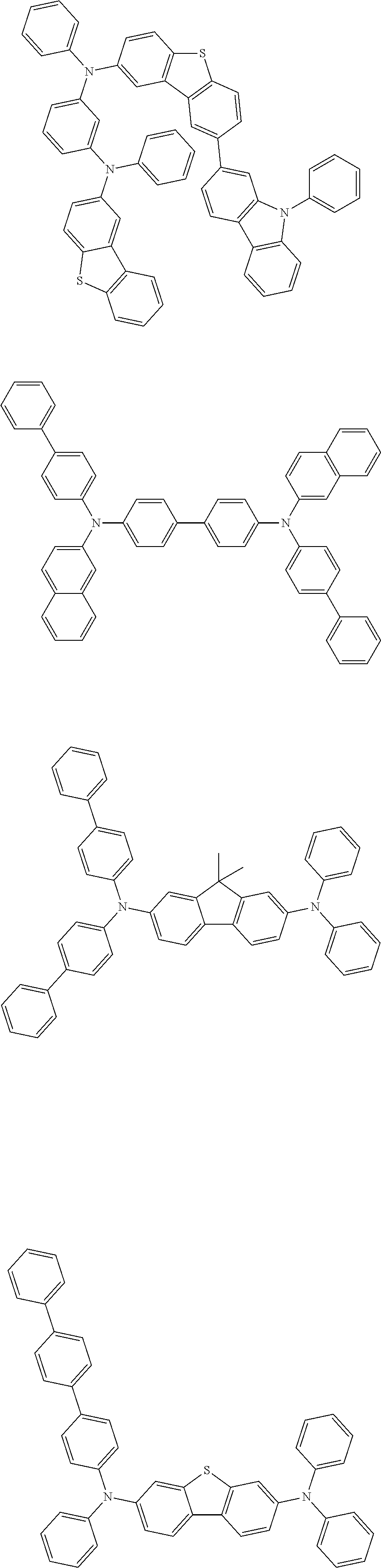 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1]

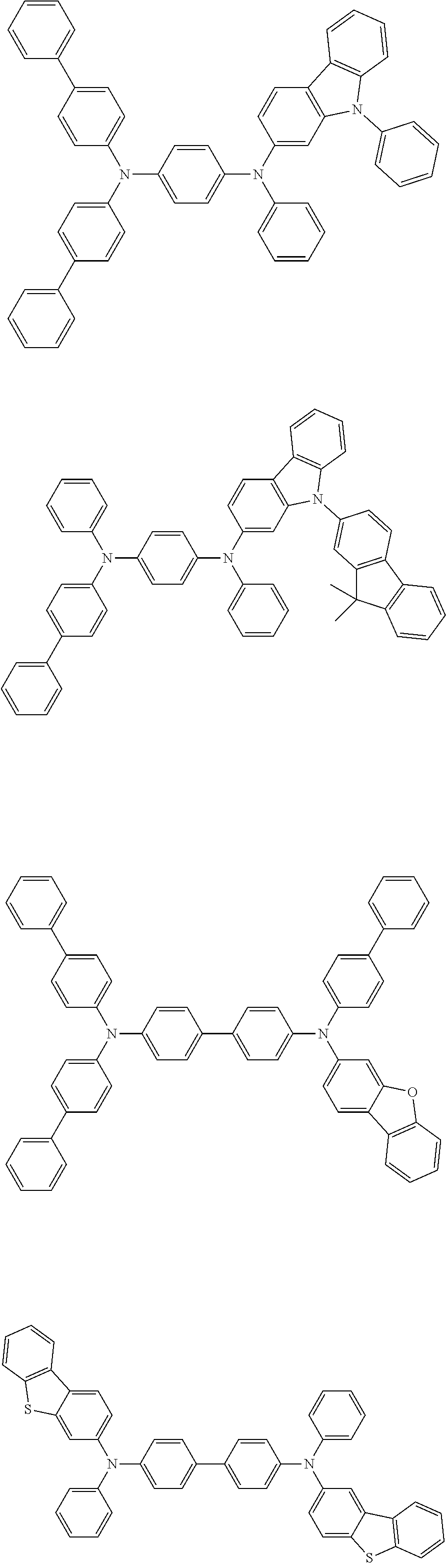 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

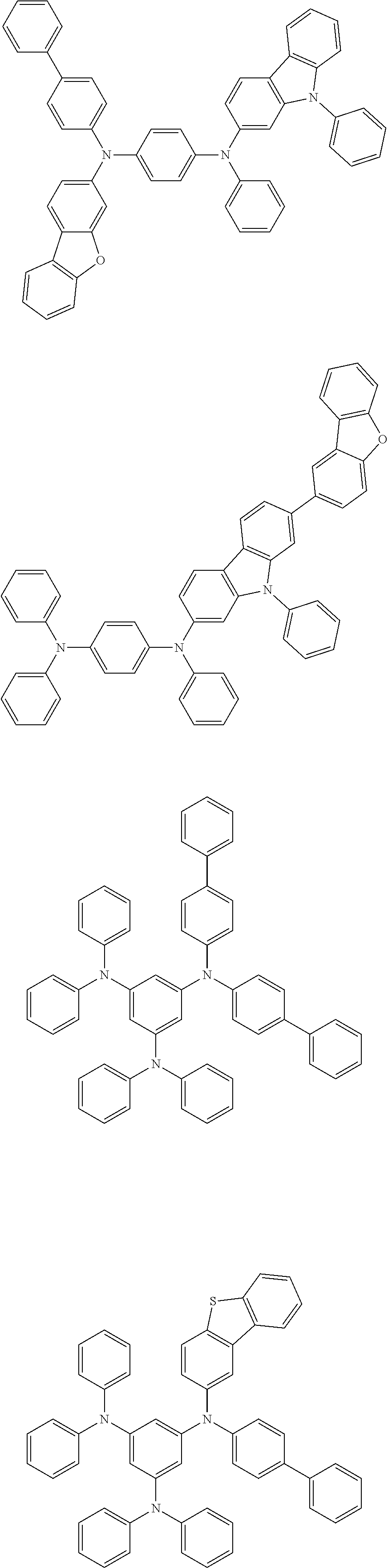 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

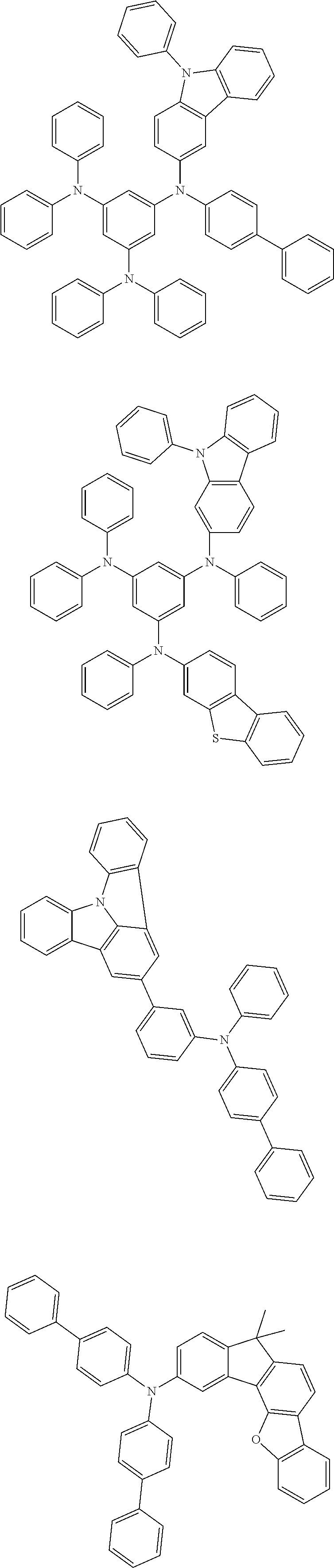 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

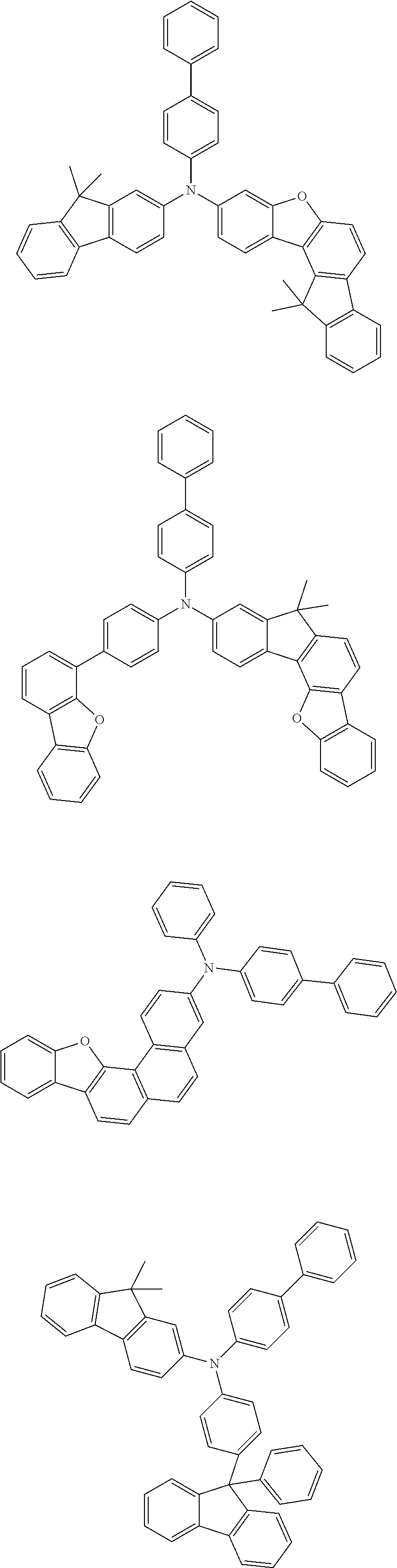 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

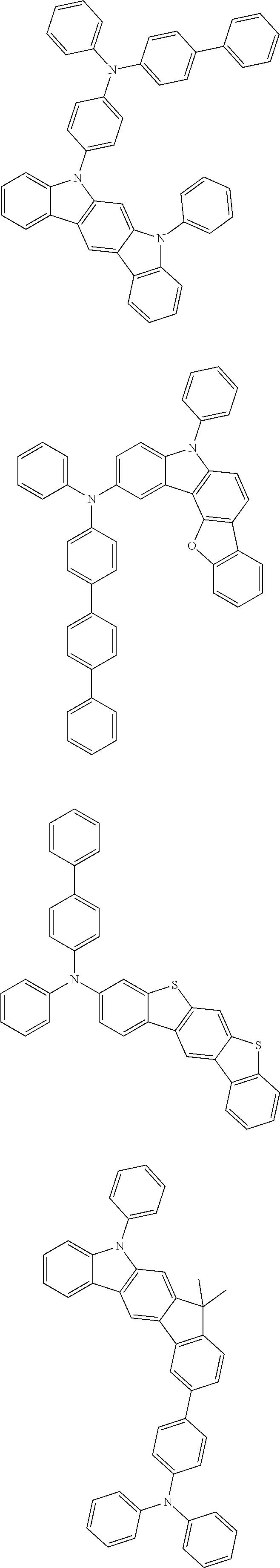 [SMILES: c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21]

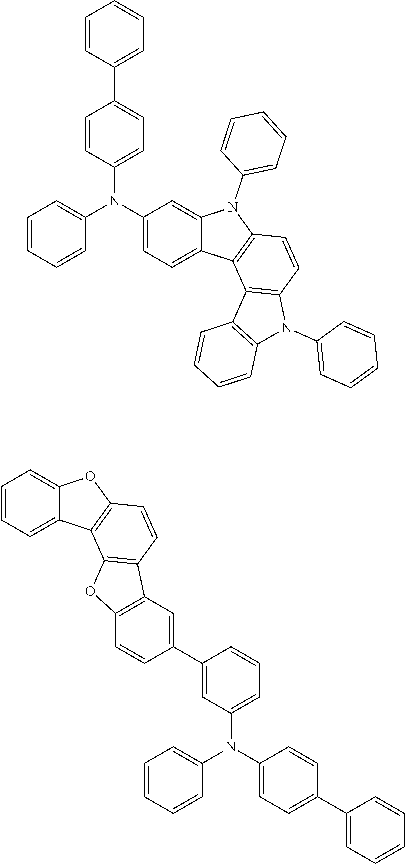 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

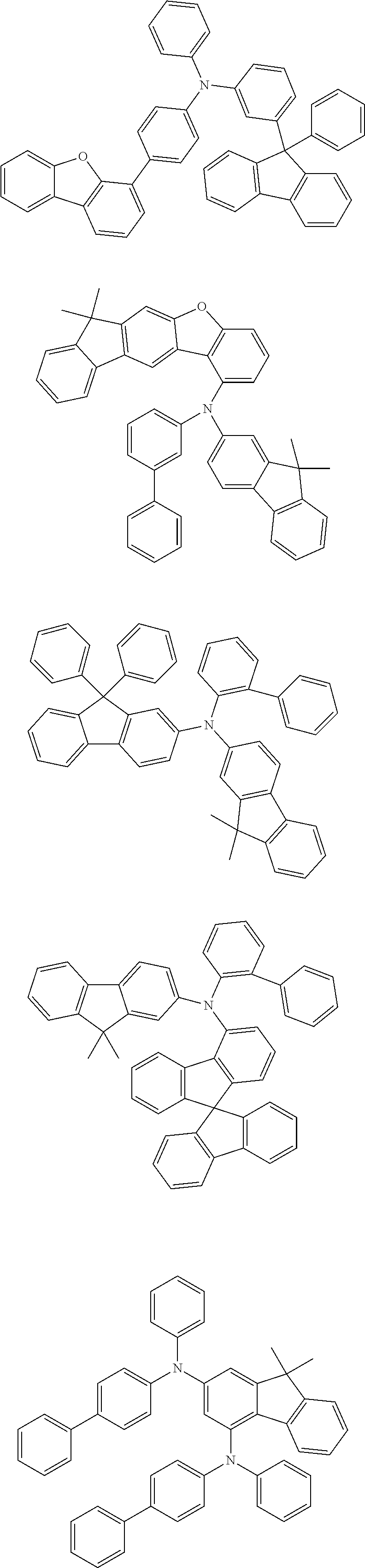 [SMILES: c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21]

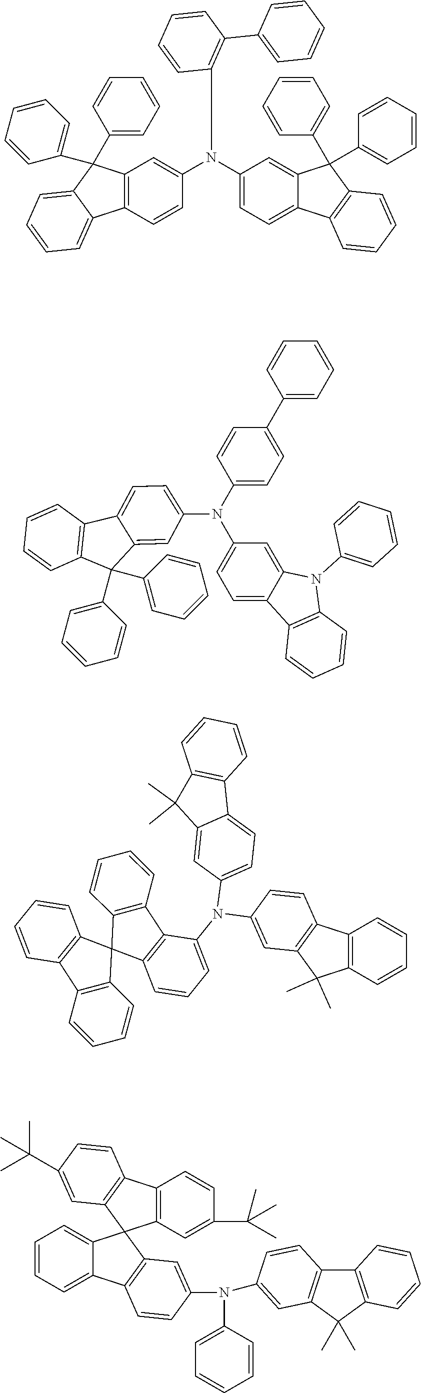 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

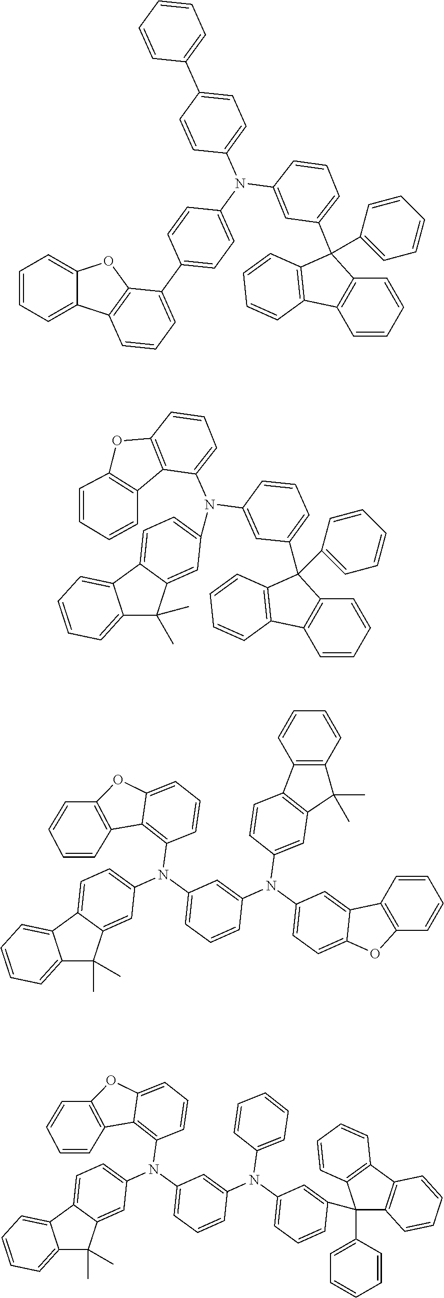 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

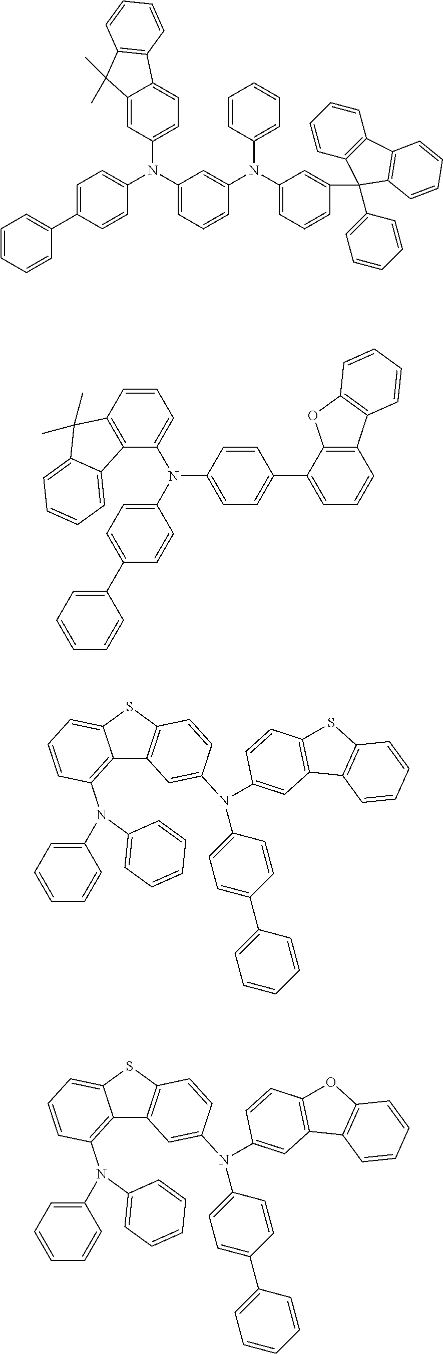 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

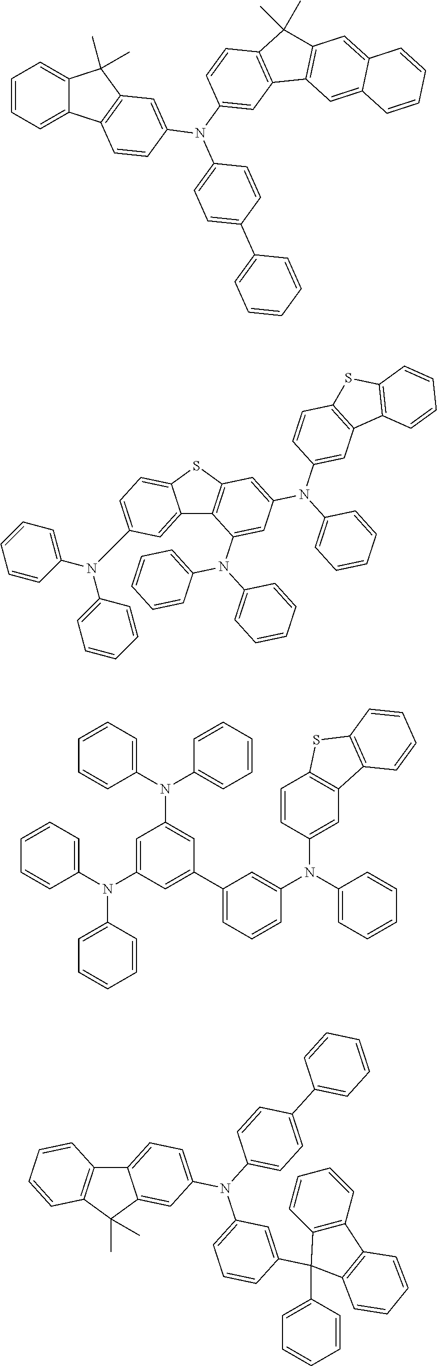 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

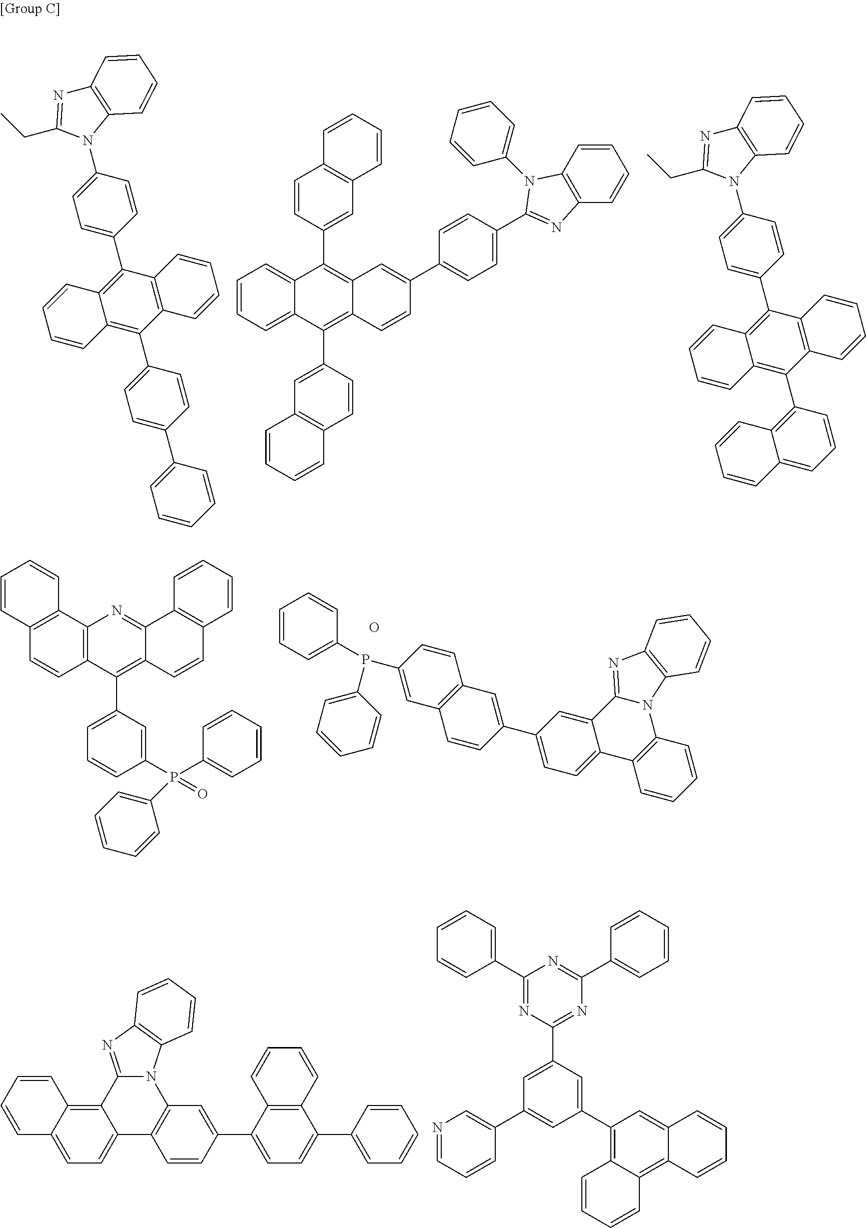

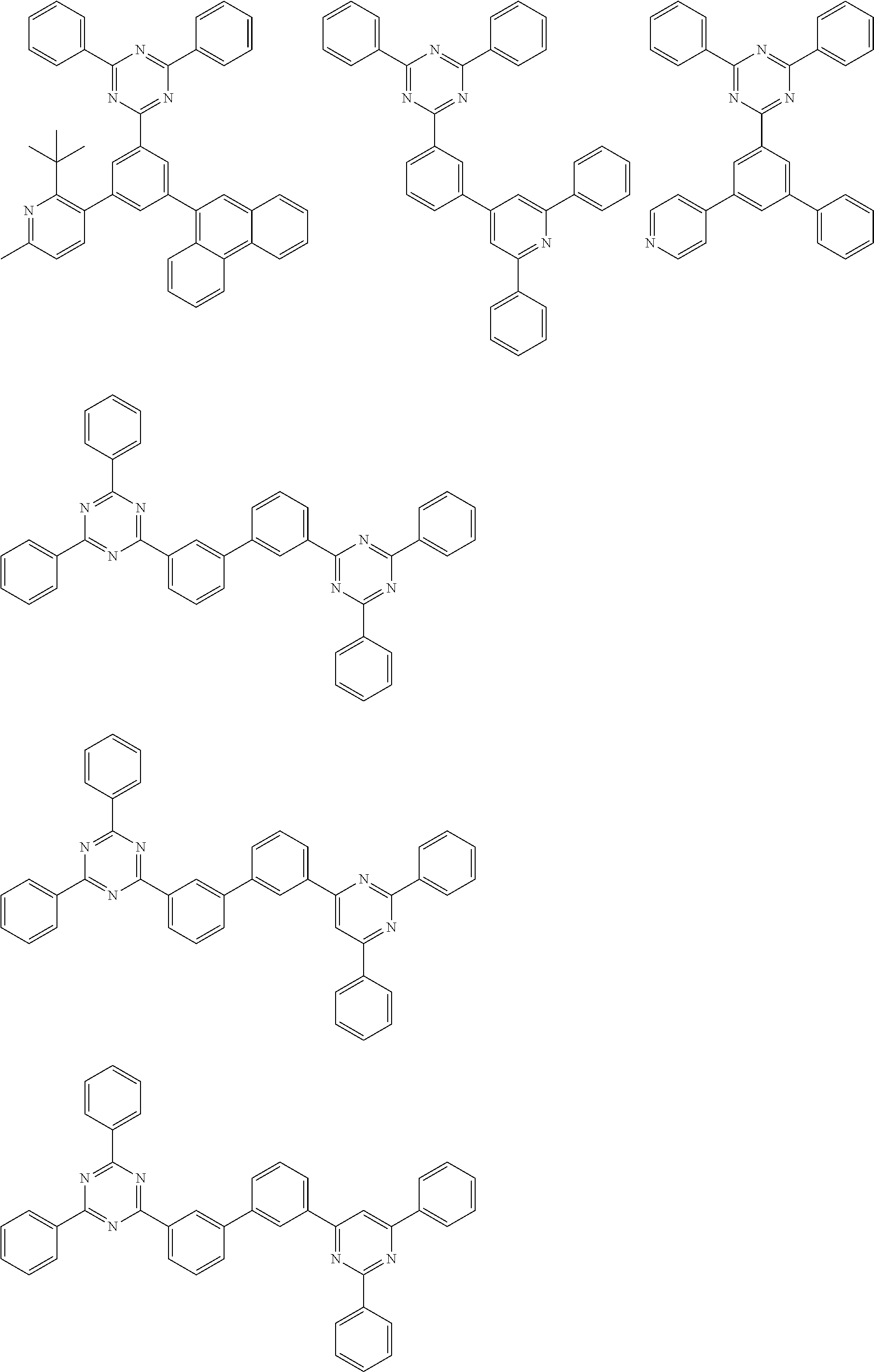

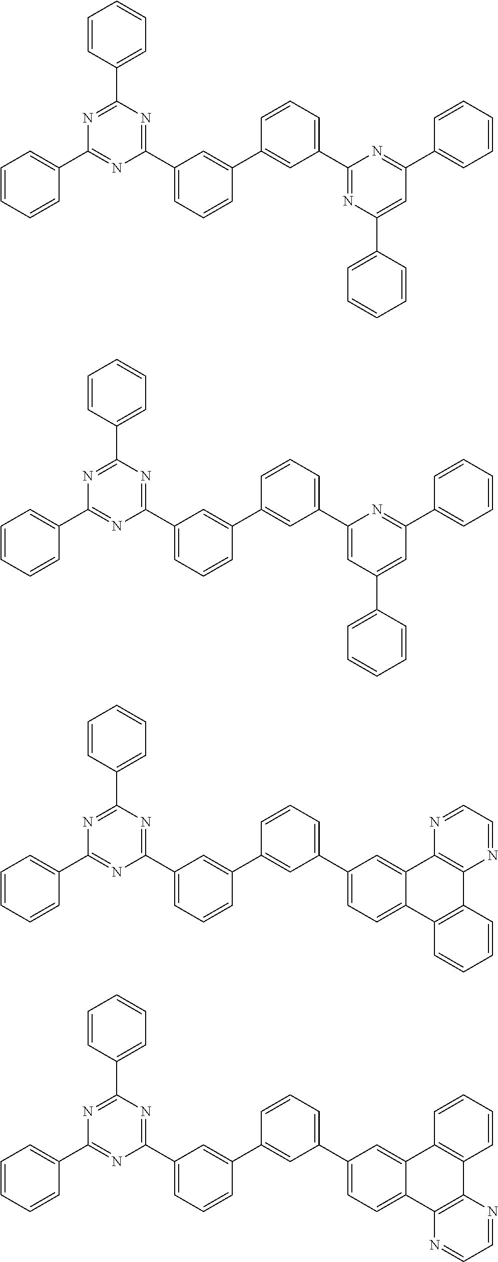 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

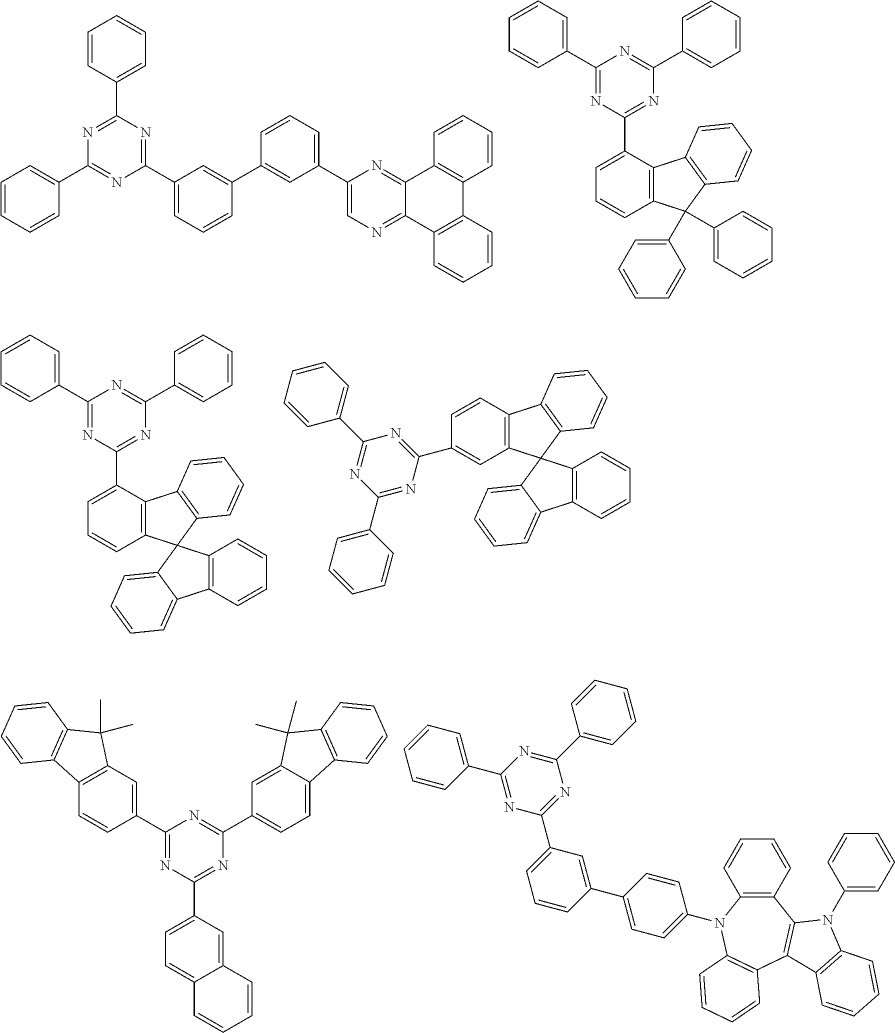

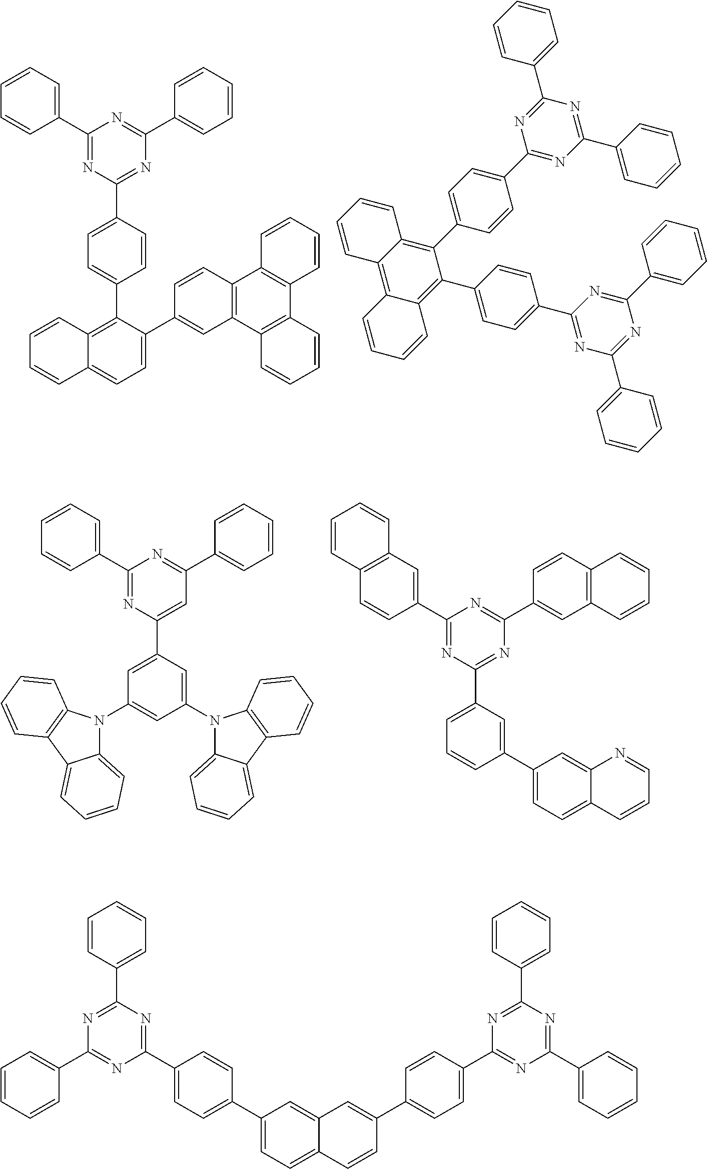

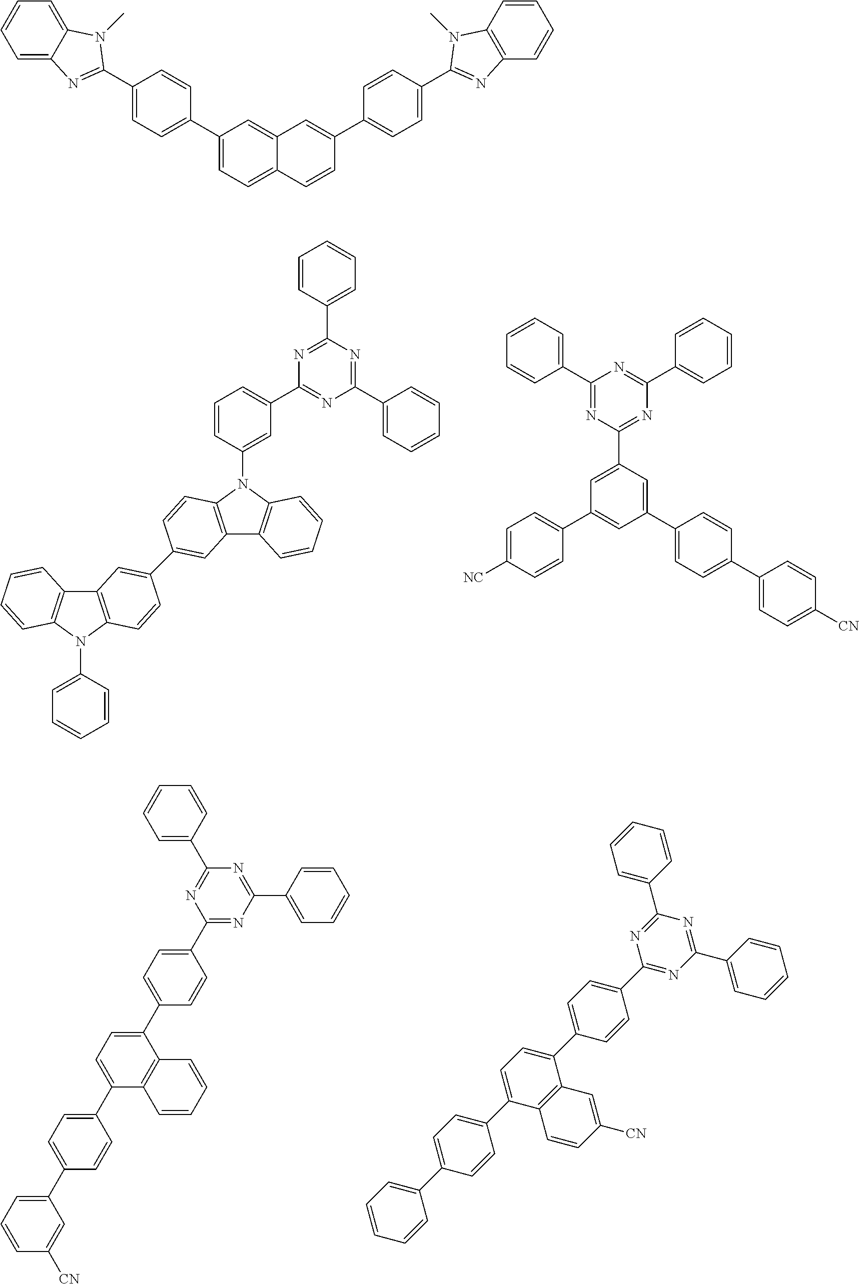 [SMILES: Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

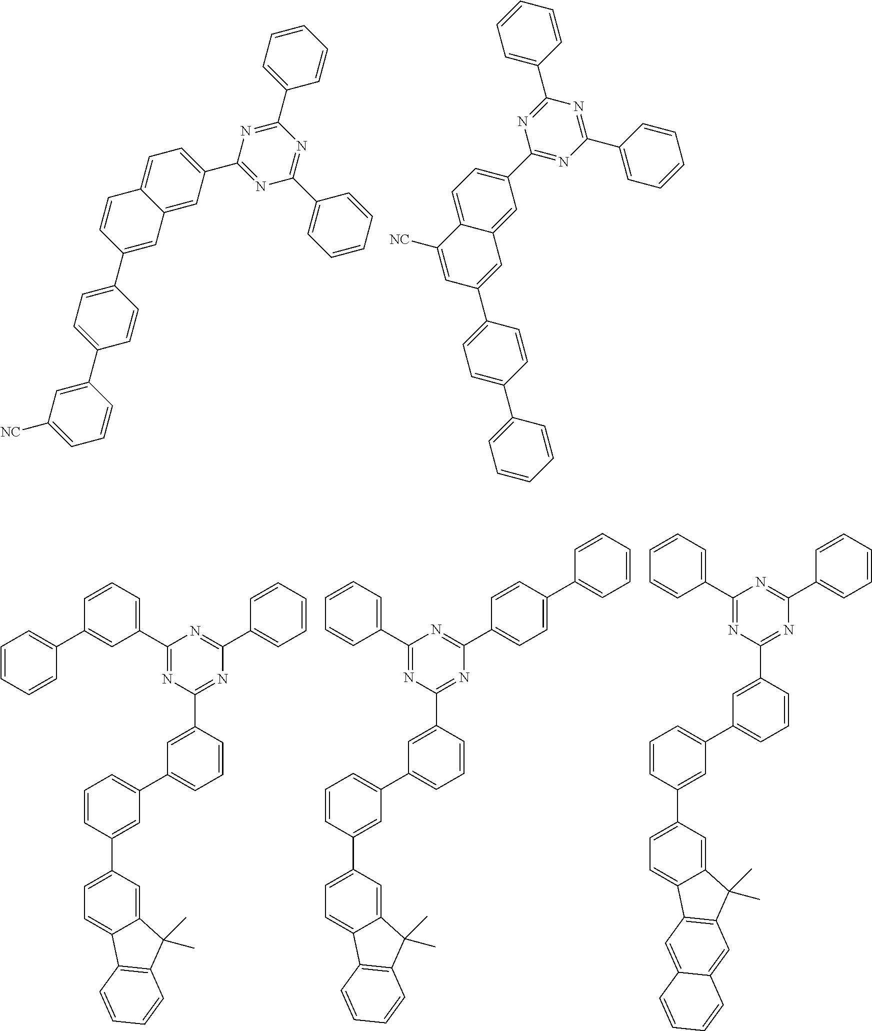

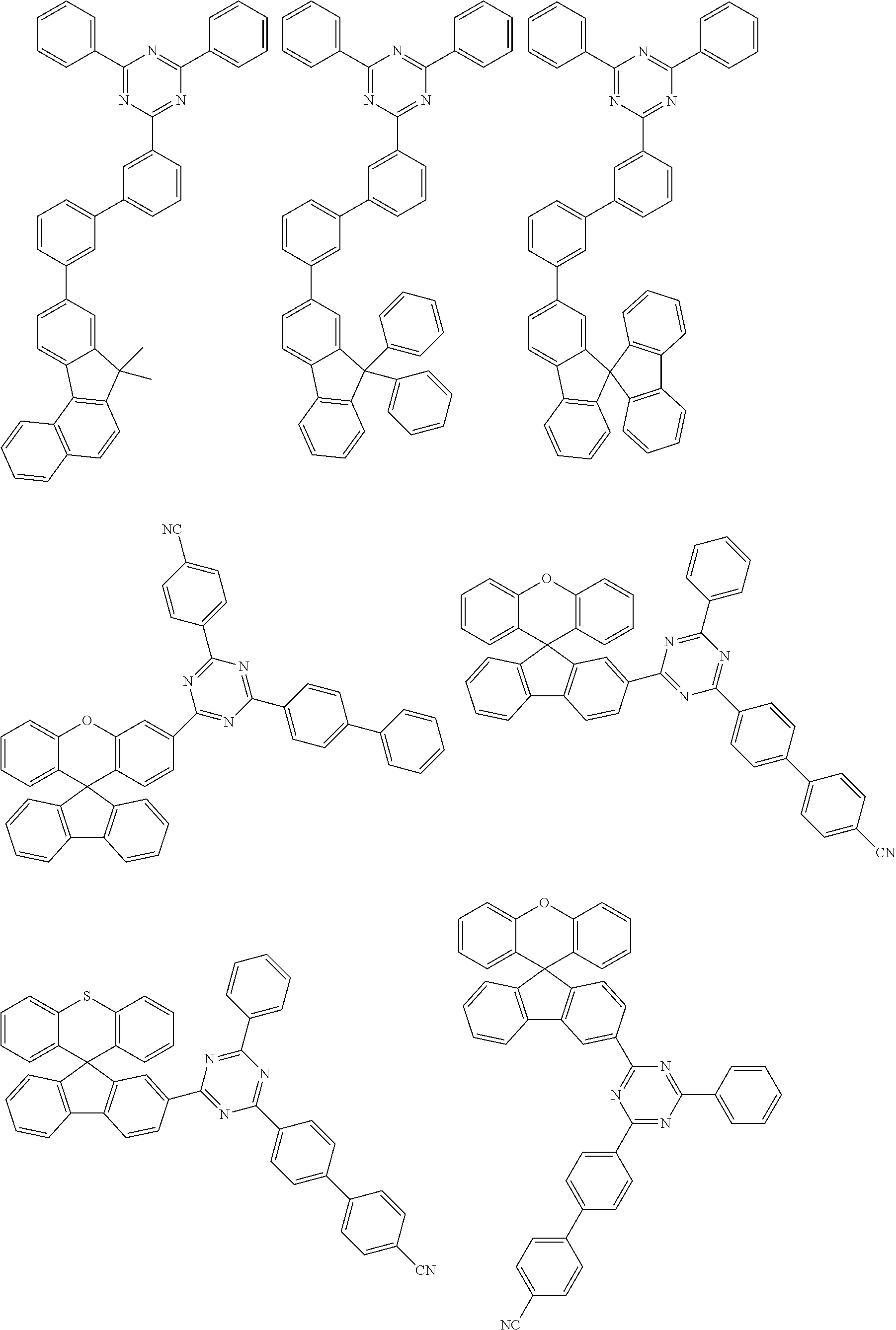

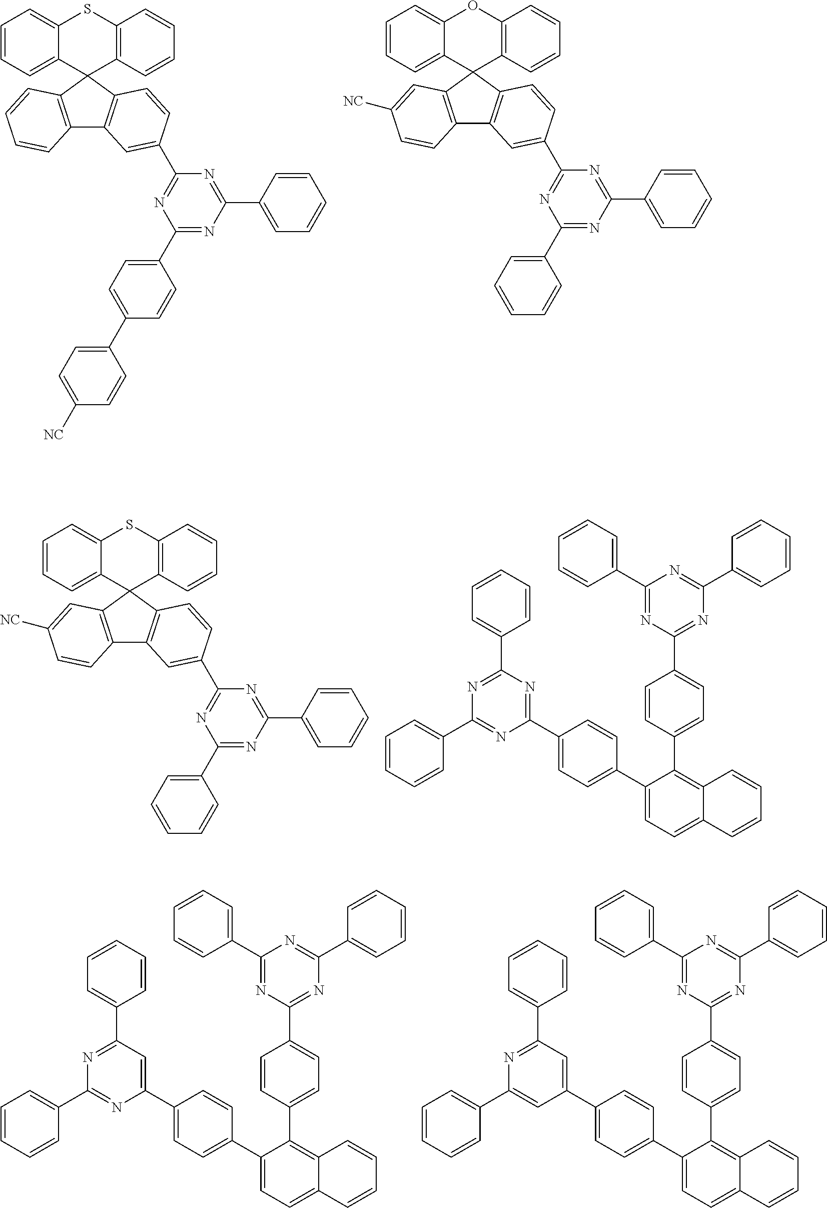

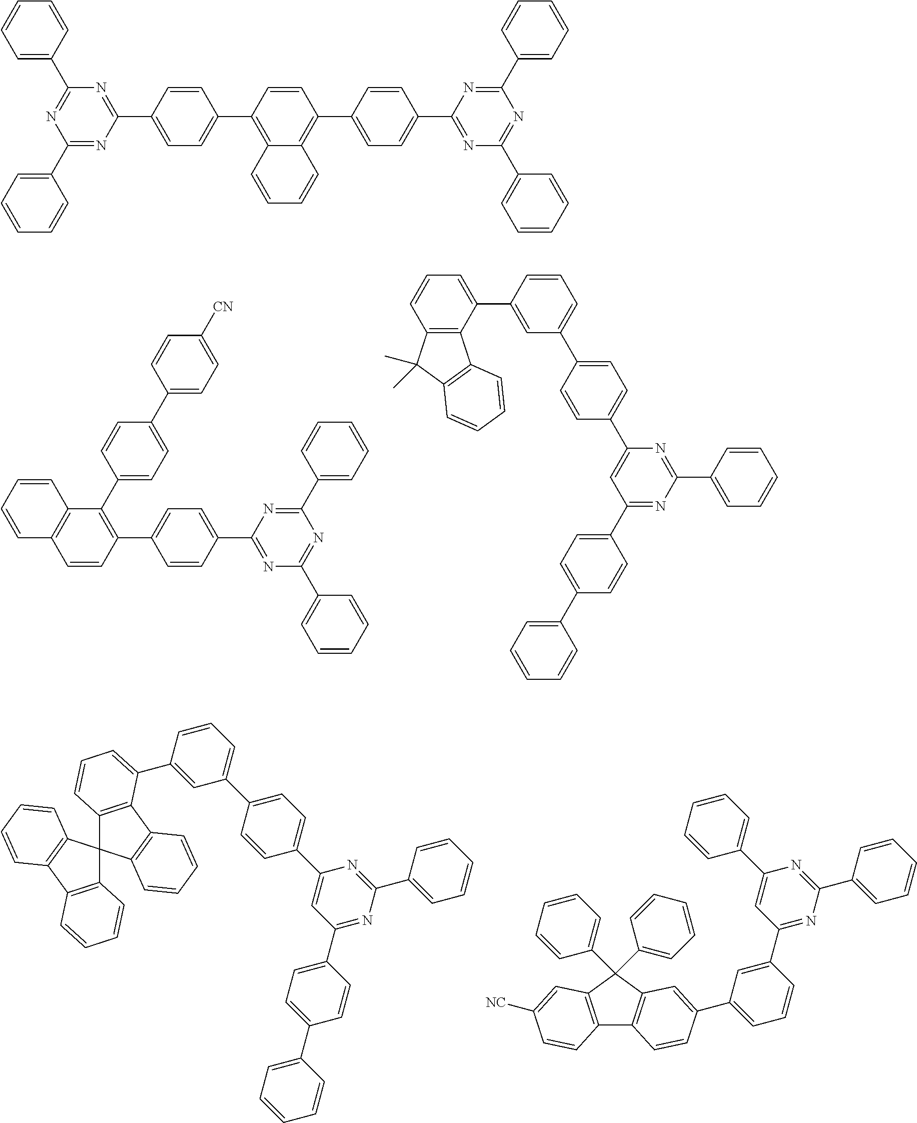

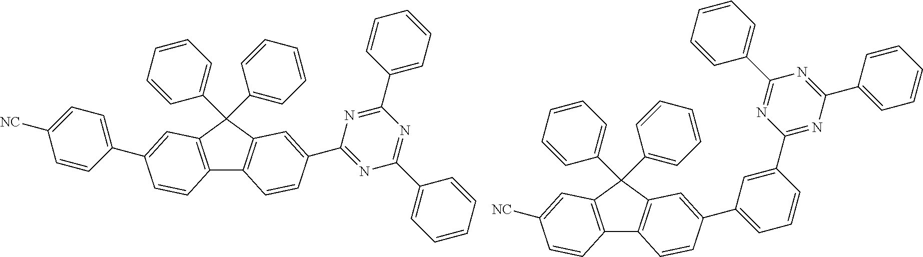

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

Another embodiment of the present invention may provide an organic light emitting diode including a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diode 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### MODE FOR INVENTION

Hereinafter, the embodiments are illustrated in more detail with reference to examples. However, these examples are exemplary, and the scope of claims is not limited thereto.

### Synthesis of First Compound

### Synthesis Example 1: Synthesis of Compound 1-2

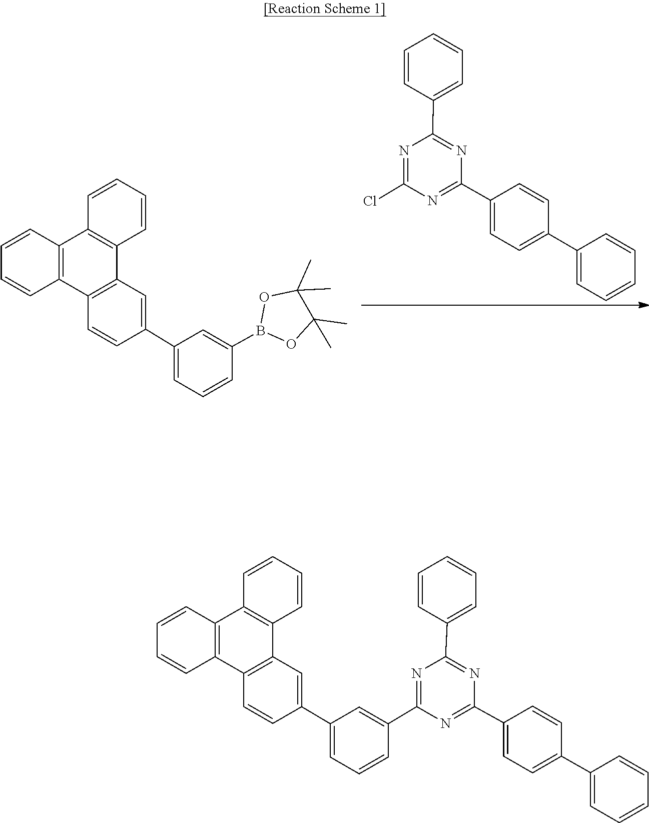 [SMILES: CC1(C)OB(c2cccc(-c3ccc4c5ccccc5c5ccccc5c4c3)c2)OC1(C)C ; Clc1nc(-c2ccccc2)nc(-c2ccc(-c3ccccc3)cc2)n1 ; c1ccc(-c2ccc(-c3nc(-c4ccccc4)nc(-c4cccc(-c5ccc6c7ccccc7c7ccccc7c6c5)c4)n3)cc2)cc1]

In a round-bottomed flask, 20.00 g (48.16 mmol) of 4,4,5,5-tetramethyl-2-(3-(triphenylene-2-yl)phenyl)-1,3,2-dioxaborolane, 17.38 g (50.56 mmol) of 2-chloro-4-(biphenyl-4-yl)-6-phenyl-1,3,5-triazine, 16.6 g (120.39 mmol) of K₂CO₃, and 1.67 g (1.67 mmol) of Pd(PPh₃)₄ were suspended in 140 ml of THE and 60 ml of distilled water under a nitrogen flow and then, stirred under reflux for 8 hours. When a reaction was completed, after separating layers, a THE layer therefrom was concentrated. The concentrated THE layer was dissolved in DCB (dichlorobenzene) and then, recrystallized to obtain a solid, which was 23 g (78%) of Compound 1-2.

### Synthesis Example 2: Synthesis of Compound 1-3

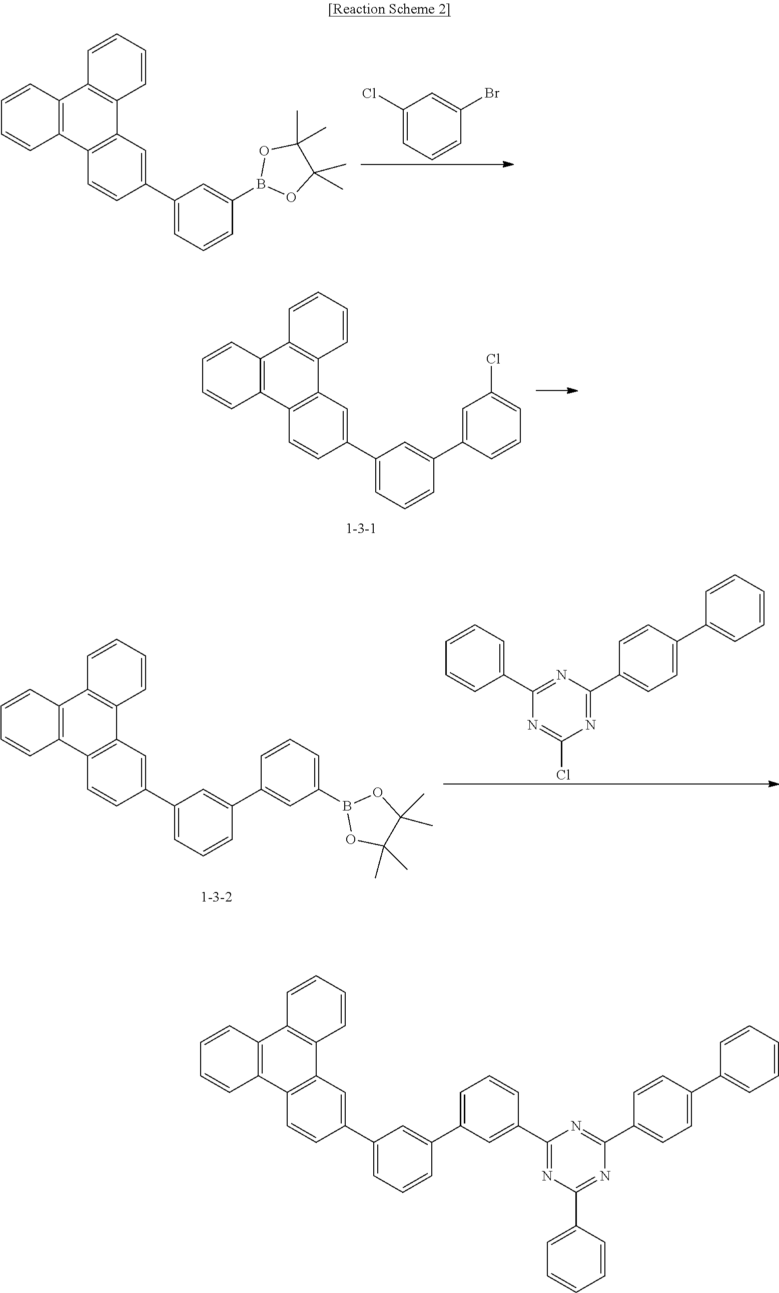 [SMILES: CC1(C)OB(c2cccc(-c3ccc4c5ccccc5c5ccccc5c4c3)c2)OC1(C)C ; Clc1cccc(Br)c1 ; Clc1cccc(-c2cccc(-c3ccc4c5ccccc5c5ccccc5c4c3)c2)c1 ; CC1(C)OB(c2cccc(-c3cccc(-c4ccc5c6ccccc6c6ccccc6c5c4)c3)c2)OC1(C)C ; Clc1nc(-c2ccccc2)nc(-c2ccc(-c3ccccc3)cc2)n1 ; c1ccc(-c2ccc(-c3nc(-c4ccccc4)nc(-c4cccc(-c5cccc(-c6ccc7c8ccccc8c8ccccc8c7c6)c5)c4)n3)cc2)cc1]

### 1st Step: Synthesis of Intermediate 1-3-1

40 g (92.95 mmol) of 4,4,5,5-tetramethyl-2-(3-(triphenylene-2-yl)phenyl)-1,3,2-dioxaborolane, 18.68 g (97.56 mmol) of 1-bromo-3-chlorobenzene, 32.12 g (232.37 mmol) of K₂CO₃, and 3.22 g (2.79 mmol) of Pd(PPh₃)₄ were suspended in 240 ml of THE and 120 ml of distilled water under a nitrogen flow and then, stirred under reflux for 8 hours. When a reaction was completed, a THE layer therefrom was concentrated to obtained a solid, which was dissolved in xylene and then, recrystallized to obtain 23 g (60%) of Intermediate 1-3-1.

### 2nd Step: Synthesis of Intermediate 1-3-2

23 g (55.43 mmol) of Intermediate 1-3-1, 21.11 g (83.15 mmol) of bis(pinacolato)diboron, 2.26 g (2.77 mmol) of Pd(dppf)Cl₂, and 10.88 g (110.86 mmol) of potassium acetate were added to a round-bottomed flask and then, dissolved in 250 ml of DMF (dimethylformamide). The mixture was stirred under reflux at 120 °C for 12 hours. When a reaction was completed, the mixture was poured into an excessive amount of distilled water and then, stirred for 1 hour. A solid was filtered therefrom and then, dissolved in xylene. After removing moisture with MgSO₄, an organic solvent was also filtered and removed under a reduced pressure by using a silica gel pad. A solid was recrystallized therefrom with xylene to obtain 26.5 g (89%) of Intermediate 1-3-2.

### 3rd Step: Synthesis of Compound 1-3

26.5 g (52.33 mmol) of Intermediate 1-3-2, 18.89 g (54.94 mmol) of 2-chloro-4-(biphenyl-4-yl)-6-phenyl-1,3,5-triazine, 18.08 g (130.82 mmol) of K₂CO₃, and 1.81 g (1.57 mmol) of Pd(PPh₃)₄ were suspended in 140 ml of THF and 70 ml of distilled water under a nitrogen flow and then, stirred under reflux for 8 hours. When a reaction was completed, MeOH was added thereto to obtain a solid, which was filtered. Subsequently, the solid was dissolved in MCB and then, filtered by using a silica gel pad to remove an organic solvent under a reduced pressure. The solid was recrystallized therefrom with MCB to obtain 26.6 g (74%) of Compound 1-3.

### Synthesis of Second Compound

### Synthesis Example 3: Synthesis of Compound B-31

It was synthesized using the same method as described in KR10-2017-0037277A.

### Synthesis Example 4: Synthesis of Compound C-5

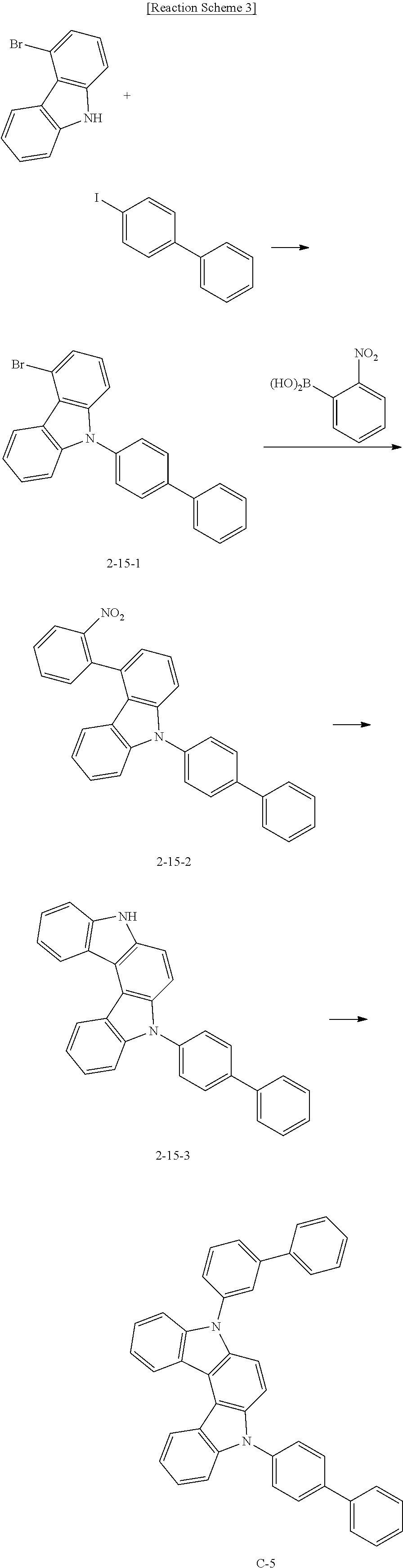

### 1st Step: Synthesis of Intermediate 2-15-1

In a round-bottomed flask, 10.44 g (42.41 mmol) of 4-bromo-9H-carbazole, 11.88 g (42.41 mmol) of 4-iodo-1,1′-biphenyl (Sigma Aldrich Co., Ltd.), 0.388 g (0.424 mmol) of Pd₂(dba)₃, 0.206 g (0.848 mmol) of P(t-Bu)₃, and 6.11 g (63.61 mmol) of NaO(t-Bu) were suspended in 420 ml of toluene and then, stirred at 60 °C for 12 hours. When a reaction was completed, distilled water was added thereto and then, stirred for 30 minutes to extract an organic layer, which was silica gel-columned (hexane/dichloromethane=9:1 (v/v)) to obtain 14.70 g (a yield: 87%) of Intermediate 2-15-1.

### 2nd Step: Synthesis of Intermediate 2-15-2

In a round-bottomed flask, 15.50 g (38.92 mmol) of the synthesized Intermediate 2-15-1, 7.15 g (42.81 mmol) of (2-nitrophenyl)-boronic acid, 16.14 g (116.75 mmol) of potassium carbonate, and 1.35 g (1.17 mmmol) of tetrakis-(triphenylphosphine)palladium (0) (Pd(PPh₃)₄) were suspended in 150 ml of toluene and 70 ml of distilled water and then, stirred under reflux for 12 hours. Subsequently, an organic layer was extracted therefrom with dichloromethane and distilled water and then, silica gel-filtered. After removing an organic solution therefrom, a solid produced therein was recrystallized with dichloromethane and n-hexane to obtain 13.72 g (a yield: 80%) of Intermediate 2-15-2.

### 3rd Step: Synthesis of Intermediate 2-15-3

22.46 g (51.00 mmol) of the synthesized Intermediate 2-15-2 and 52.8 ml of triethyl phosphite were added to a round-bottomed flask and after internally substituting it with nitrogen, stirred at 160 °C for 12 hours. When a reaction was completed, 3 L of MeOH was added thereto and then, filtered to volatilize its filtrate. Then, 10.42 g (a yield: 50%) of Intermediate 2-15-3 was obtained by purification through column chromatography (hexane).

### 4th Step: Synthesis of Compound C-5

Compound C-5 was synthesized (a yield: 60%) in the same manner as in the 1^{st} step of Synthesis Example 4 except that Intermediate 2-15-3 and 3-iodo-1,1′-biphenyl were used. (LC/MS: theoretical value 560.23 g/mol, measured value: 561.57 g/mol)

### Synthesis Example 5: Synthesis of Compound B-186

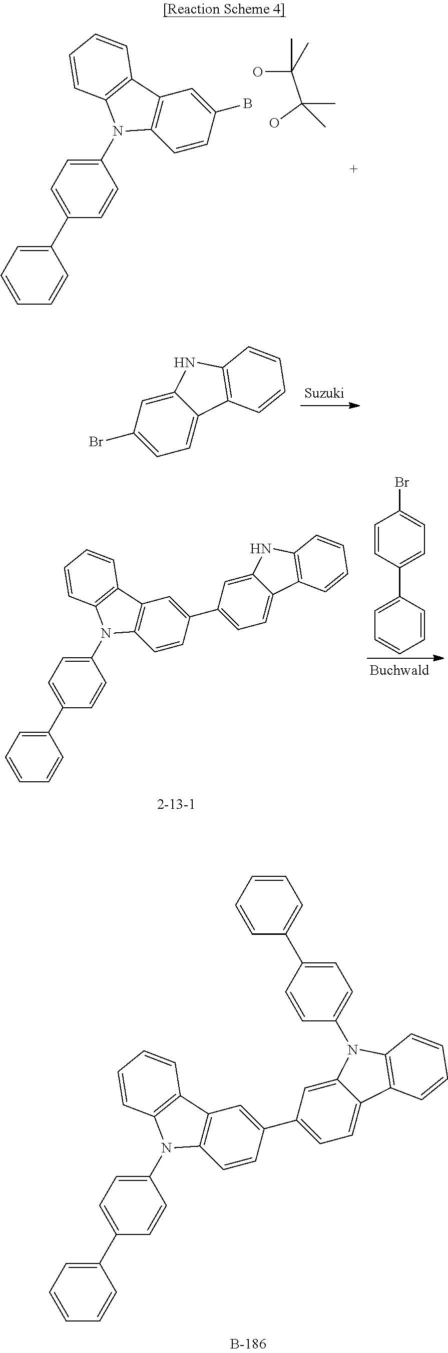

### 1st Step: Synthesis of Intermediate 2-13-1

In a round-bottomed flask, 18.23 g (40.94 mmol) of 2-[9-([1,1′-biphenyl]-4-yl)-9H-carbazole-3-yl]-4,4,5,5-tetramethyl-1,3,2-dioxaborolane, 11.08 g (45.03 mmol) of 2-bromo-9H-carbazole, 11.32 g (81.88 mmol) of potassium carbonate, and 1.42 g (1.23 mmmol) of tetrakis-(triphenylphosphine)palladium (0) (Pd(PPh₃)₄) were suspended in 180 ml of tetrahydrofuran (THF) and 75 ml of distilled water and then, stirred under reflux. Subsequently, an organic layer was extracted therefrom with dichloromethane and distilled water and then, silica gel-filtered. After removing an organic solution, a solid obtained therefrom was recrystallized with dichloromethane and n-hexane to obtain 18.05 g (a yield: 91%) of Intermediate 2-13-1.

### 2nd Step: Synthesis of Compound B-186

In a round-bottomed flask, 13.29 g (27.42 mmol) of Intermediate 2-13-1, 6.39 g (27.42 mmol) of 4-bromobiphenyl, 0.25 g (0.274 mmol) of Pd₂(dba)₃, 0.133 g (0.274 mmol) of P(t-Bu)₃, and 3.95 g (41.13 mmol) of NaO(t-Bu) were suspended in 300 ml of toluene and then, stirred 60 °C for 12 hours. When a reaction was completed, distilled water was added thereto and then, stirred for 30 minutes to extract an organic layer, which was silica gel-columned (hexane/dichloromethane=9:1 (v/v)) to obtain 15.37 g (a yield: 88%) of Compound B-186.

LC-Mass (theoretical value: 636.26 g/mol, measured value: M+=637.40 g/mol)

### Synthesis of Third Compound

### Synthesis Example 6: Synthesis of Compound 3-6

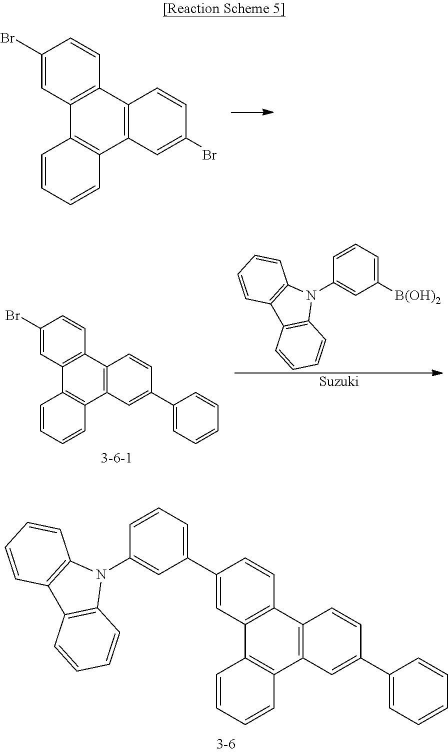 [SMILES: Brc1ccc2c3ccc(Br)cc3c3ccccc3c2c1 ; Brc1ccc2c3ccc(-c4ccccc4)cc3c3ccccc3c2c1 ; OB(O)c1cccc(-n2c3ccccc3c3ccccc32)c1 ; c1ccc(-c2ccc3c4ccc(-c5cccc(-n6c7ccccc7c7ccccc76)c5)cc4c4ccccc4c3c2)cc1]

### 1st Step: Synthesis of Intermediate 3-6-1

Intermediate 3-6-1 was synthesized in the same method as described in KR2014135532 except that 2,7-dibromotriphenylene (888041-37-0) was used.

### 2nd Step: Synthesis of Intermediate 3-6

1 equivalent of Intermediate 3-6-1 (65.23 mmol), 1.1 equivalent of an intermediate (3-(9H-carbazol-9-yl)phenyl)boronic acid (cas: 864377-33-3) (71.75 mmol), 2.26 g (1.96 mmol) of Pd(PPh₃)₄, and 22.54 g (163.07 mmol) of K₂CO₃ were suspended in 200 ml of THF and 100 ml of distilled water and then, stirred under reflux for 12 hours. When a reaction was completed, after cooling the resultant to room temperature, a solid produced therefrom was filtered and then, washed with distilled water and acetone. The solid was dissolved by heating in 200 ml of dichlorobenzene, filtered with silica gel, and then, recrystallized in 500 ml of dichlorobenzene to synthesize Compound 3-6 with a yield of 80%.

LC-Mass (theoretical value: 545.21 g/mol, measured value: M+=546.27 g/mol)

### Synthesis Example 7: Synthesis of Compound 3-7

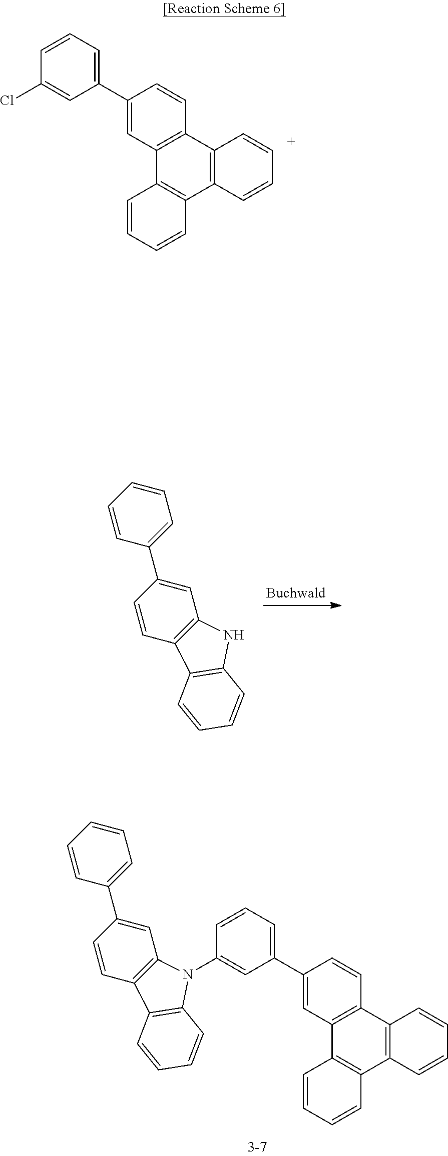 [SMILES: Clc1cccc(-c2ccc3c4ccccc4c4ccccc4c3c2)c1 ; c1ccc(-c2ccc3c(c2)[nH]c2ccccc23)cc1 ; c1ccc(-c2ccc3c4ccccc4n(-c4cccc(-c5ccc6c7ccccc7c7ccccc7c6c5)c4)c3c2)cc1]

### 1st Step: Synthesis of 3-7

Compound 3-7 was synthesized with a yield of 85% in the same manner as in the 2^{nd} step of Synthesis Example 5 except that 1 equivalent of 2-(3-chlorophenyl)-triphenylene (cas: 1384206-44-3) and 1.05 equivalent of 2-phenyl-9H-carbazole (cas:88590-00-5) were used.

LC-Mass (theoretical value: 545.21 g/mol, measured value: M+=546.24 g/mol)

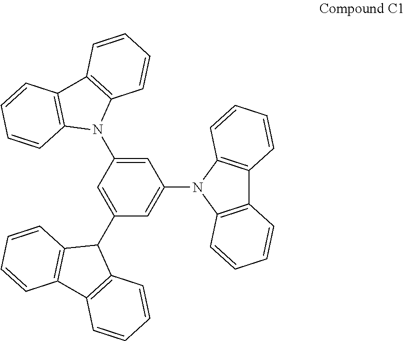 [SMILES: c1ccc2c(c1)-c1ccccc1C2c1cc(-n2c3ccccc3c3ccccc32)cc(-n2c3ccccc3c3ccccc32)c1]

### Synthesis of Dopant

### Synthesis Example 8: Synthesis of Dopant Compound PtGD

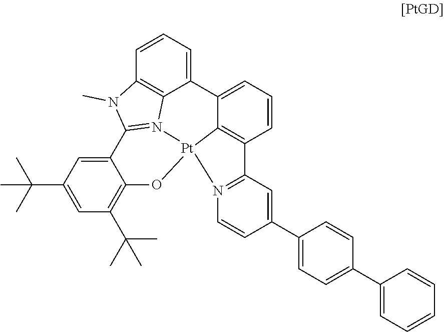

It was synthesized using the same method described in KR1999337.

### Manufacture of Organic Light Emitting Diode

### Example 1

A glass substrate coated with a thin film of ITO (indium tin oxide) was cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound B was deposited on the hole transport layer to a thickness of 350 Å to form a hole transport auxiliary layer. Compound 1-3, Compound 2-2, and Compound 3-7 were simultaneously used as hosts on the hole transport auxiliary layer, and 15 wt % of PtGD was doped as a dopant to form a 400 Å-thick light emitting layer by vacuum deposition. Here, Compound 1-3, Compound 2-2, and Compound 3-7 were used in a weight ratio of 30:55:15, and the ratios were described separately for the following examples and comparative examples. Then, Compound C was deposited on the light emitting layer to a thickness of 50 Å to form an electron transport auxiliary layer, and Compound D and LiQ were simultaneously vacuum deposited at a weight ratio of 1:1 to form an electron transport layer to a thickness of 300 Å. An organic light emitting diode was manufactured by sequentially vacuum-depositing 15 Å of LiQ and 1,200 Å of Al on the electron transport layer to form a cathode.

ITO/Compound A (3% NDP-9 doping, 100 Å)/Compound A (1,350 Å)/Compound B (350 Å)/EML [1-3: 2-2:3-7 [PtGD] (15 wt %)](400 Å)/Compound C (50 Å)/Compound D: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N,N-bis(9,9-dimethyl-9H-fluoren-4-yl)-9,9-spirobi(fluorene)-2-amine
Compound C: 2-[3′-(9,9-dimethyl-9H-fluoren-2-yl)[1,1′-biphenyl]-3-yl]-4,6-diphenyl-1,3,5-triazine
Compound D: 2-[4-[4-(4′-cyano-1,1′-biphenyl-4-yl)-1-naphthyl]phenyl]-4,6-diphenyl-1,3,5-triazine

### Examples 2 and 3

Each organic light emitting diode was manufactured in the same manner as in Example 1 except that the composition was changed to hosts shown in Tables 1 to 3.

### Comparative Examples 1 to 7

Each organic light emitting diode was manufactured in the same manner as in Example 1 except that the composition was changed to hosts shown in Tables 1 to 3.

### Evaluation

The organic light emitting diodes according to Examples 1 to 3 and Comparative Examples 1 to 7 were measured with respect to efficiency and life-span.

The measurement was performed in the following method, and the results are shown in Tables 1 to 3.

### (1) Life-Span Measurement

A life-span was obtained by measuring time that current efficiency (cd/A) was reduced to 95%, while maintaining luminance (cd/m²) of 24,000 cd/m². The results are shown as a relative life-span ratio based on life-spans of Comparative Examples 1, 4, and 6.

### (2) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (3) Measurement of Luminance Change Depending on Voltage Change

Luminance was measured by using a luminance meter (Minolta Cs-1000A), while the voltage of the organic light emitting diodes was increased from 0 V to 10 V.

### (4) Measurement of Current Efficiency

Current efficiency (cd/A) for required luminance of 9000 nit was calculated by using the luminance, current density, and voltage measured in (1) and (2). The Current efficiency (cd/A) was shown as a relative efficiency ratio based on current efficiency of Comparative Examples 1, 4, and 6.

TABLE 1

| | | | | | | Life- |
|---|---|---|---|---|---|---|
| | | | | | Effi- | span |
| | | | | First host:second | ciency | ratio |
| | First | Second | Third | host:third host | ratio | T95 |
| | host | host | host | ratio (wt:wt) | (%) | (%) |
| Example 1 | 1-3 | B-31 | 3-7 | 30:55:15 | 100 | 122 |
| Comparative | 1-3 | B-31 | — | 35:65:0 | 100 | 100 |
| Example 1 | | | | | | |
| Comparative | 1-3 | — | 3-7 | 35:0:65 | 85 | 45 |
| Example 2 | | | | | | |
| Comparative | 1-3 | B-31 | C1 | 30:55:15 | 98 | 90 |
| Example 3 | | | | | | |

TABLE 2

| | | | | | | Life- |
|---|---|---|---|---|---|---|
| | | | | | Effi- | span |
| | | | | First host:second | ciency | ratio |
| | First | Second | Third | host:third host | ratio | T95 |
| | host | host | host | ratio (wt:wt) | (%) | (%) |
| Example 2 | 1-2 | C-5 | 3-6 | 35:55:10 | 100 | 115 |
| Comparative | 1-2 | C-5 | — | 35:65:0 | 100 | 100 |
| Example 4 | | | | | | |
| Comparative | 1-2 | — | 3-6 | 35:0:65 | 80 | 48 |
| Example 5 | | | | | | |

TABLE 3

| | | | | | | Life- |
|---|---|---|---|---|---|---|
| | | | | | Effi- | span |
| | | | | First host:second | ciency | ratio |
| | First | Second | Third | host:third host | ratio | T95 |
| | host | host | host | ratio (wt:wt) | (%) | (%) |
| Example 3 | 1-2 | B-186 | 3-7 | 32:48:20 | 100 | 135 |
| Comparative | 1-2 | B-186 | — | 35:65:0 | 100 | 100 |
| Example 6 | | | | | | |
| Comparative | 1-2 | — | 3-7 | 35:0:65 | 80 | 50 |
| Example 7 | | | | | | |

Referring to Tables 1 to 3, the organic light emitting diodes of Examples 1 to 3, compared with the organic light emitting diodes of Comparative Examples 1 to 7, were confirmed to exhibit significantly improved life-span as well as maintained equivalent or higher efficiency.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

1. A composition for an organic optoelectronic device, comprising:
a first compound;
a second compound; and
a third compound,
wherein the first compound is represented by Chemical Formula 1,
the second compound is represented by Chemical Formula 2 or a combination of Chemical Formula 3 and Chemical Formula 4, and
the third compound is represented by Chemical Formula 5: 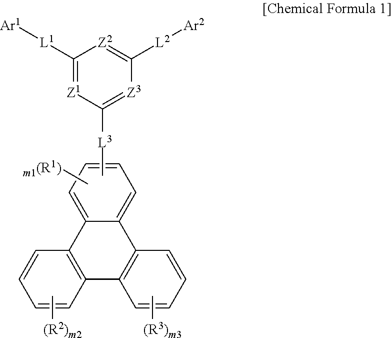
wherein, in Chemical Formula 1,
Z¹ to Z³ are N or C-L^{a}-R^{a},
at least two of Z¹ to Z³ are N,
L^{a} and L¹ to L³ are each independently a single bond, a substituted or unsubstituted C6 to C20 arylene group, a substituted or unsubstituted C2 to C20 heterocyclic group, or a combination thereof,
Ar¹ and Ar² are each independently a substituted or unsubstituted C6 to C30 aryl group, a substituted or unsubstituted C2 to C30 heterocyclic group, or a combination thereof,
R^{a} and R¹ to R³ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
each R^{a} is independently present or is linked to Ar¹ or Ar² to form a substituted or unsubstituted heteroaromatic polycyclic ring,
R¹ to R³ are each independently present or adjacent groups thereof are linked to form a substituted or unsubstituted heteroaromatic polycyclic ring,
m1 is an integer of 1 to 3, and
m2 and m3 are each independently an integer of 1 to 4; 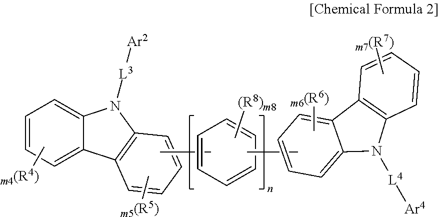
wherein, in Chemical Formula 2,
Ar³ and Ar⁴ are each independently a substituted or unsubstituted C6 to C30 aryl group or a substituted or unsubstituted C2 to C30 heterocyclic group,
L³ and L⁴ are each independently a single bond or a substituted or unsubstituted C6 to C20 arylene group,
R⁴ to R⁸ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
m5 and m6 are each independently an integer of 1 to 3,
m4, m7, and m8 are each independently an integer of 1 to 4, and
n is an integer of 0 to 2; 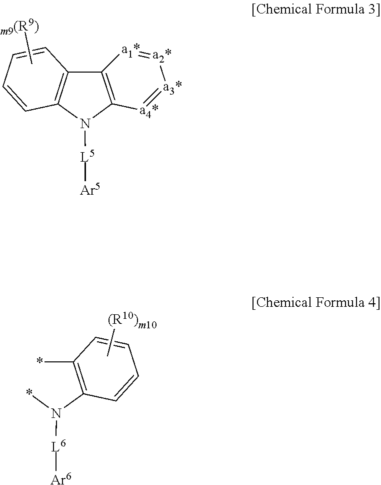
wherein, in Chemical Formulas 3 and 4,
Ar⁵ and Ar⁶ are each independently a substituted or unsubstituted C6 to C30 aryl group or a substituted or unsubstituted C2 to C30 heterocyclic group,
a₁* to a₄* in Chemical Formula 3 are each independently a linking carbon or C-L^{b}-R^{b},
among a₁* to a₄* in Chemical Formula 3, two adjacent ones are linked at * of Chemical Formula 4,
L^{b}, L⁵, and L⁶ are each independently a single bond or a substituted or unsubstituted C6 to C20 arylene group,
R^{b}, R⁹, and R¹⁰ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group, and
m9 and m10 are each independently an integer of 1 to 4; 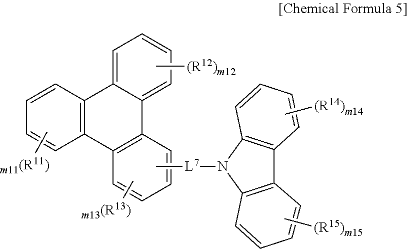
wherein, in Chemical Formula 5,
R¹¹ to R¹⁵ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
L⁷ is a single bond or a substituted or unsubstituted C6 to C20 arylene group,
m11, m12, m14, and m15 are each independently an integer of 1 to 4, and
m13 is an integer of 1 to 3.

2. The composition for the organic optoelectronic device as claimed in claim 1, wherein:
the first compound is represented by one of Chemical Formula 1-1 to Chemical Formula 1-3: 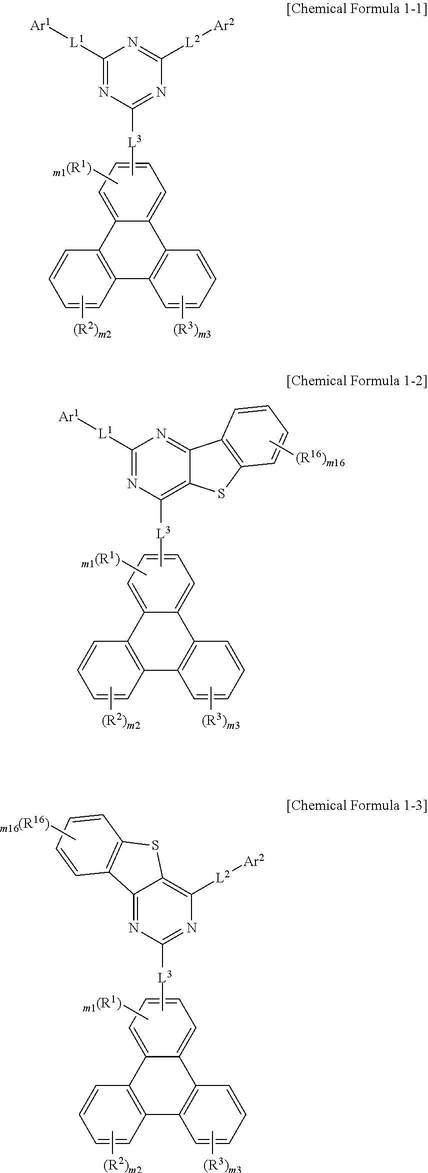
in Chemical Formula 1-1 to Chemical Formula 1-3,
R¹ to R³, L¹ to L³, Ar¹, Ar², and m1 to m3 are defined the same as those of Chemical Formula 1,
each R¹⁶ is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and
m16 is an integer of 1 to 4.

3. The composition for the organic optoelectronic device as claimed in claim 1, wherein:
the first compound is represented by one of Chemical Formula 1-4 to Chemical Formula 1-6: 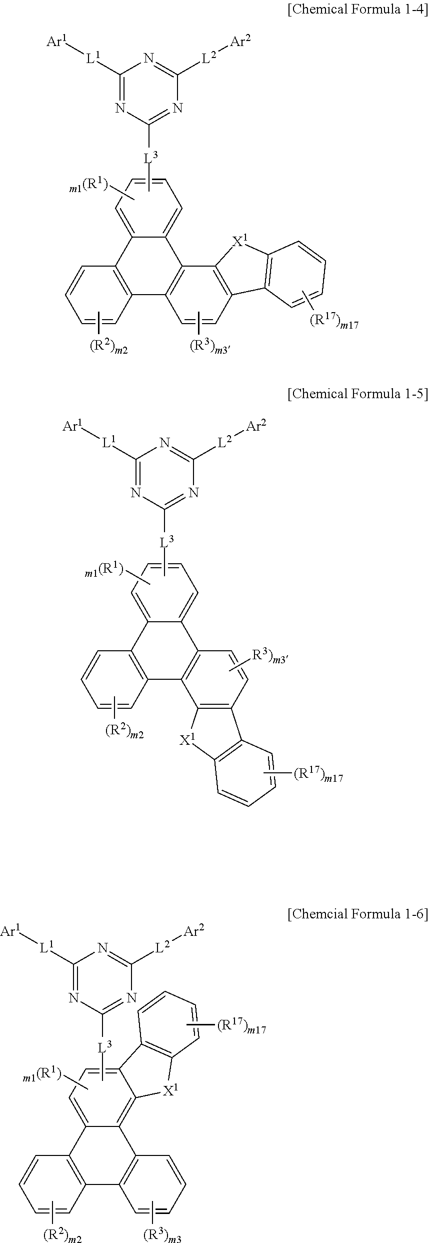
in Chemical Formula 1-4 to Chemical Formula 1-6,
Z¹ to Z³, R¹ to R³, L¹ to L³, Ar¹, Ar² and m1 to m3 are defined the same as those of Chemical Formula 1,
X¹ is O or S,
each R¹⁷ is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
m17 is an integer of 1 to 4,
m1′ is 1, and
m3′ is an integer of 1 or 2.

4. The composition for the organic optoelectronic device as claimed in claim 1, wherein:
the first compound is represented by Chemical Formula 1-A: 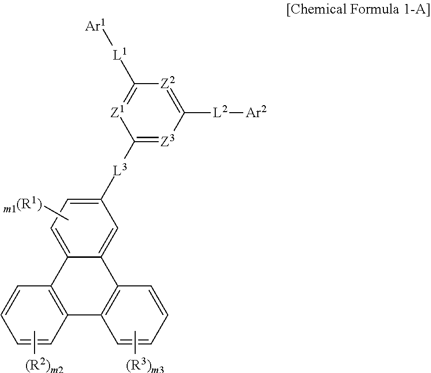
in Chemical Formula 1-A, Z¹ to Z³, L¹ to L³, R¹ to R³, L¹ to L³, Ar¹, Ar², and m1 to m3 are defined the same as those of Chemical Formula 1.

5. The composition for the organic optoelectronic device as claimed in claim 1, wherein:
the second compound is represented by one of Chemical Formula 2-6, Chemical Formula 2-8, and Chemical Formula 3C: 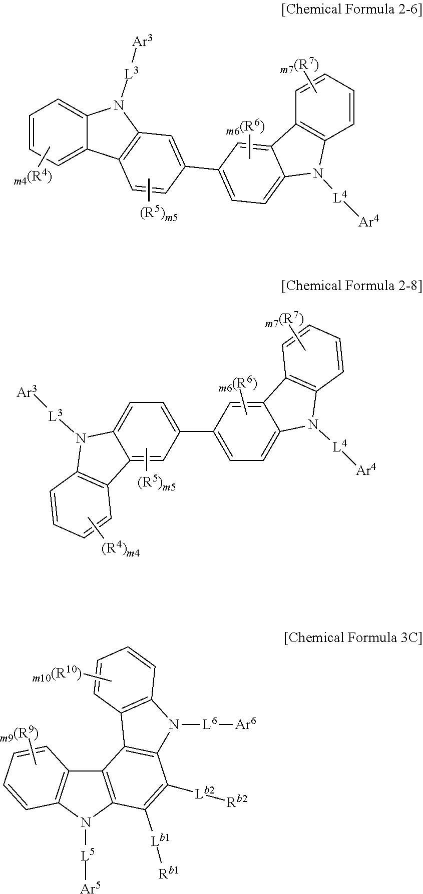
in Chemical Formula 2-6, Chemical Formula 2-8, and Chemical Formula 3C,
L³ to L⁶, Ar³ to Ar⁶, R⁴ to R⁷, R⁹, R¹⁰ and m4 to m7, m8, and m9 are defined the same as those of Chemical Formula 2 and Chemical Formulas 3 and 4,
L^{b1} and L^{b2} are defined the same as L^{b} of Chemical Formula 3, and
R^{b1} and R^{b2} are defined the same as R^{b} of Chemical Formula 3.

6. The composition for the organic optoelectronic device as claimed in claim 1, wherein in Chemical Formula 5, L⁷ is a single bond, a substituted or unsubstituted phenylene group, a substituted or unsubstituted biphenylene group, or a substituted or unsubstituted terphenylene group.

7. The composition for the organic optoelectronic device as claimed in claim 1, wherein:
in Chemical Formula 5, L⁷ is a single bond or a linking group of Group II: 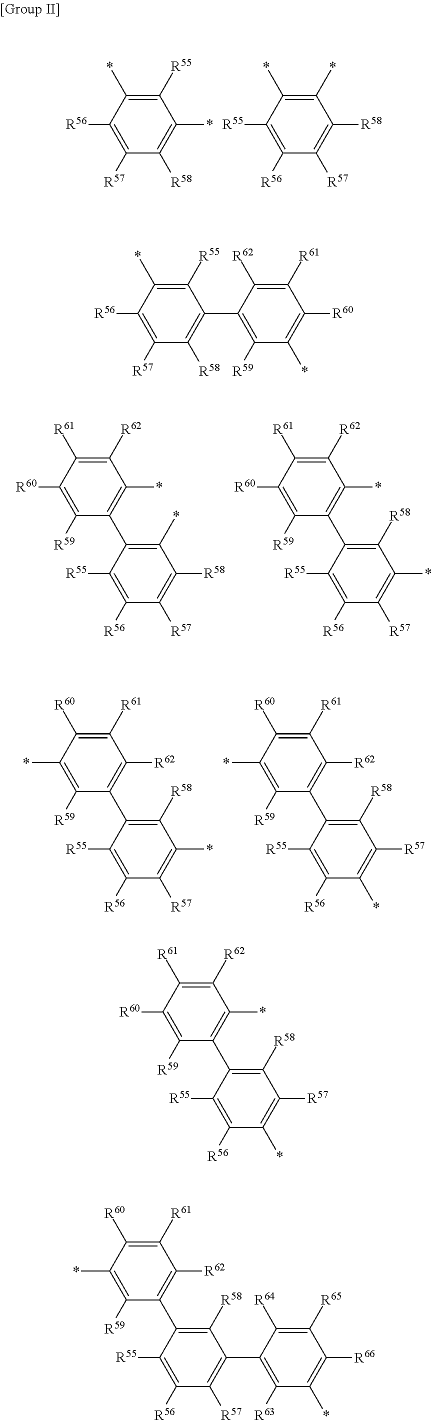 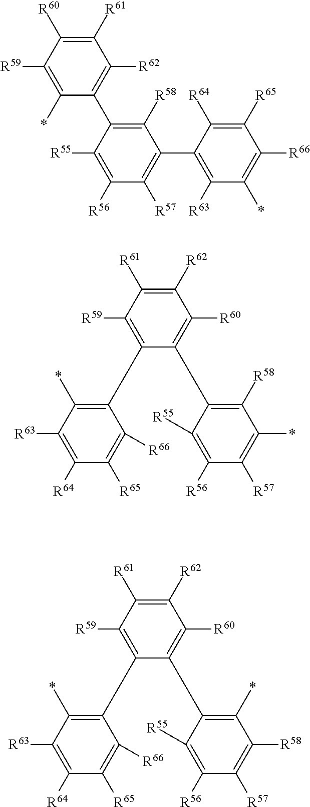
in Group II,
R⁵⁵ to R⁶⁶ are each independently hydrogen, deuterium, a C1 to C10 alkyl group, a substituted or unsubstituted C6 to C12 aryl group, a halogen, a cyano group, or a combination thereof, and
* is a linking point.

8. The composition for the organic optoelectronic device as claimed in claim 1, wherein:
the third compound is represented by one of Chemical Formula 5-I to Chemical Formula 5-III: 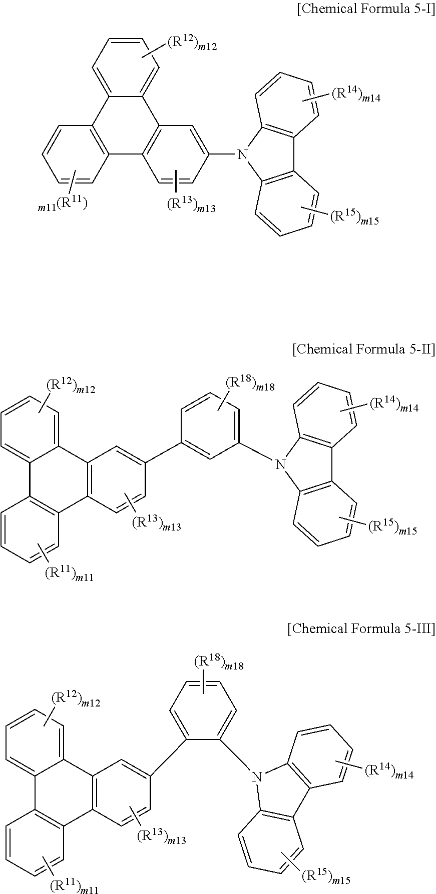
in Chemical Formula 5-I to Chemical Formula 5-III, R¹¹ to R¹⁵, R¹⁸ and m11 to m15 are defined the same as those of Chemical Formula.

9. The composition for the organic optoelectronic device as claimed in claim 1, wherein in Chemical Formula 5, R¹¹ to R¹³ are each independently hydrogen, deuterium, a substituted or unsubstituted phenyl group, or a substituted or unsubstituted biphenyl group.

10. The composition for the organic optoelectronic device as claimed in claim 1, wherein in Chemical Formula 5, R¹⁴ and R¹⁵ are each independently hydrogen, deuterium, a substituted or unsubstituted phenyl group, a substituted or unsubstituted biphenyl group, a substituted or unsubstituted carbazolyl group, a substituted or unsubstituted dibenzofuranyl group, or a substituted or unsubstituted dibenzothiophenyl group.

11. The composition for the organic optoelectronic device as claimed in claim 1, wherein;
the first compound is represented by Chemical Formula 1-A, and
the second compound is represented by one of Chemical Formula 2-6, Chemical Formula 2-8, and Chemical Formula 3C, and
the third compound is represented by Chemical Formula 5-II: 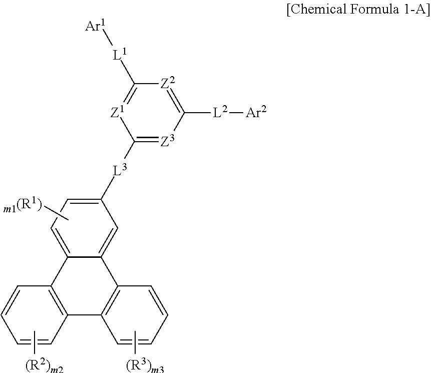
in Chemical Formula 1-A,
Z¹ to Z³, L¹ to L³, R¹ to R³, L¹ to L³, Ar¹, Ar², and m1 to m3 are defined the same as those of Chemical Formula 1; 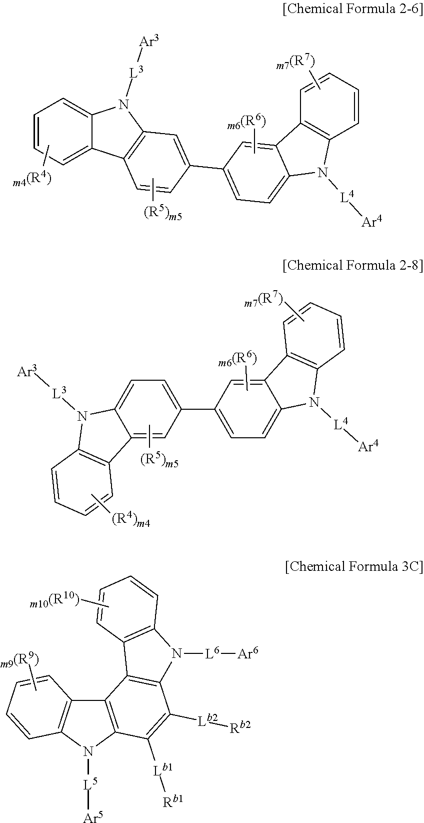
in Chemical Formula 2-6, Chemical Formula 2-8, and Chemical Formula 3C, R⁴ to R⁷, R⁹, R¹⁰, m4 to m7, m9, and m10 are defined the same as those of Chemical Formula 2 and Chemical Formulas 3 and 4; 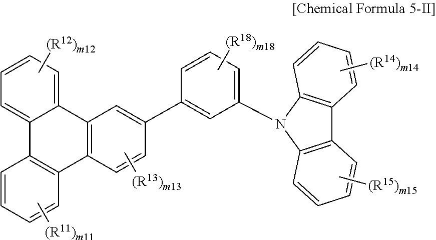
in Chemical Formula 5-II,
R¹¹ to R¹⁵, R¹⁸ and m11 to m15 are defined the same as those of Chemical Formula 5, and
m18 is an integer of 1 to 4.

12. An organic optoelectronic device, comprising;
an anode and a cathode facing each other, and
at least one organic layer between the anode and the cathode,
wherein the at least one organic layer includes the composition for an organic optoelectronic device of claim 1.

13. The organic optoelectronic device as claimed in claim 12, wherein:
the at least one organic layer includes a light emitting layer, and
the light emitting layer includes the composition.

14. The organic optoelectronic device as claimed in claim 13, wherein the first compound, the second compound, and the third compound are each included as a phosphorescent host of the light emitting layer.

15. The organic optoelectronic device as claimed in claim 14, wherein:
the first compound is included in an amount of about 20 wt % to 50 wt %, based on a total weight of the first compound, the second compound, and the third compound,
the second compound is included in an amount of about 40 wt % to 60 wt %, based on a total weight of the first compound, the second compound, and the third compound, and
the third compound is included in an amount of about 10 wt % to 30 wt %, based on a total weight of the first compound, the second compound, and the third compound.

16. The organic optoelectronic device as claimed in claim 13, wherein the composition for the organic optoelectronic device further includes a dopant.

17. The organic optoelectronic device as claimed in claim 16, wherein the composition for the organic optoelectronic device is a green light emitting composition.

18. A display device comprising the organic optoelectronic device as claimed in claim 12.
